(19) United States
(12) Patent Application Publication
(10) Pub. No.: US 2024/0132475 A1
(43) Pub. Date: Apr. 25, 2024

(54) SUBSTITUTED PYRAZOLYL COMPOUNDS AS MALT-1 INHIBITORS

(71) Applicant: AURIGENE ONCOLOGY LIMITED, Bangalore, Karnaka (IN)

(72) Inventors: Dinesh CHIKKANNA, Bangalore, Karnaka (IN); Susanta SAMAJDAR, Bangalore, Karnaka (IN); Srinivasa Raju SAMMETA, Bangalore, Karnaka (IN); Sunil Kumar PANIGRAHI, Boudh, Odisha (IN)

(73) Assignee: AURIGENE ONCOLOGY LIMITED, Bangalore, Karnaka (IN)

(21) Appl. No.: 18/264,076

(22) PCT Filed: Feb. 3, 2022
(86) PCT No.: PCT/IB2022/050934

§ 371 Date: Aug. 3, 2023

(30) Foreign Application Priority Data
Feb. 4, 2021 (IN) 20214100469

(51) Int. Cl. C07D 401/14; C07D 401/12; C07D 403/12; C07D 405/14; C07D 409/14

(52) U.S. Cl. CPC C07D 401/14; C07D 401/12; C07D 403/12; C07D 405/14; C07D 409/14

(57) ABSTRACT

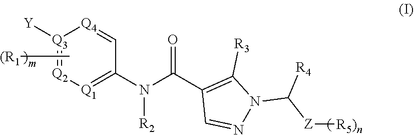

The present invention is directed to compound of formula (I) and, a pharmaceutically acceptable salt or a stereoisomer thereof that useful as MALT-1 inhibitors for the treatment of diseases or disorders dependent on MALT-1. The present invention also relates to a method of preparation of the said compounds and pharmaceutical compositions comprising the said compounds.

### RELATED APPLICATION

This application claims the benefit of Indian provisional patent application number 202141004769, filed on Feb. 4, 2021; the contents of which are hereby incorporated by reference in their entirety.

### FIELD OF THE INVENTION

The present invention relates to substituted pyrazolyl compounds, a pharmaceutically acceptable salt thereof or a stereoisomer thereof which are useful as MALT-1 inhibitors for the treatment of diseases or disorders dependent on MALT-1. The present invention also relates to a method of preparation of the said pyrazolyl compounds, pharmaceutical compositions comprising them and their uses as therapeutic agents for the treatment of diseases, particularly cancer and autoimmune diseases.

### BACKGROUND OF THE INVENTION

MALT-1 (mucosa associated lymphoid tissue lymphoma translocation protein 1) is an intracellular signalling protein known from innate (natural killer cells NK, dendritic cells DC, and mast cells) and adaptive immune cells (T cells and B cells) (WO2018141749). MALT-1, in association with BCL10 and CARD11, functions as a scaffolding protein to activate the inhibitor of IκB kinase (IKK) complex (L. Fontan et al., Clin Cancer Res; 2013 Dec 15;19(24):pp. 6662-8). The function of MALT-1 is best known in the context of T cell receptor (TCR signalling), where it mediates nuclear factor κB (NF-κB) signalling leading to control of lymphocyte activation, survival, and differentiation (A. Demeyer et al., Trends Mol Med. 2016 Feb. 22(2), pp. 135-150). In addition to its scaffolding function, MALT-1 is the only gene encoding a paracaspase that belongs to the caspase (cysteine-aspartic proteases) family of proteases that displays high homology to caspases from mammals and metacaspases from plants and fungi (A. G. Uren et al., Mol. Cell, 6 (2000), pp. 961-967). The paracaspase MALT-1 plays an essential role in the activation of immune cells by specific subtypes of immune receptors, which induce nuclear translocation of the NFκB transcription factor complex leading to the activation of the NF-κB signalling pathway NF-κB target genes include cytokines and anti-apoptotic proteins, which together promote the activation, proliferation and survival of the activated immune cells upon receptor triggering, and thereby allow the efficient generation of an immune response (M. Jaworski et al., Cell Mol. Life Science 2016, 73, pp. 459-473).

Studies in BCL10 and MALT 1 deficient mice seem to suggest their essential role in the signaling cascade from the antigen receptors to the transcription factor NFkB. Moreover, chromosomal translocations leading to overexpression of BCL10 and MALT-1 or creating the constitutively active fusion protein API2-MALT-1, appear to result in an uncontrolled and stimulus-independent activation of NFkB. Inhibitors of the proteolytic activity of MALT-1 have been described with activity in preclinical lymphoma models (M. Vincendeau et al. Int. J. Hematol. Oncol. 2013, 2, 409-417, WO2017081641). Moreover, certain publications appear to suggest the important role of MALT-1 and its proteolytic function in signaling cascades triggered by innate cell receptors like Dectin receptors and in signaling cascades triggered by G-protein coupled receptors in many cell types.

International applications WO2017081641, WO2018020474, WO2018119036, WO2018165385, WO2018226150, WO2015181747, WO2017057695, WO2018141749, WO2019243964, WO2019243965, WO2020169736, WO2020169738, WO2020208222, WO2021000855, WO2021134004, WO2021138298, WO2021207343, WO2021241611 and WO2020111087 report several small molecule compounds and their derivatives capable of targeting MALT-1. However, there is a need for the development of small molecule MALT-1 inhibitors with the potential to dampen NF-kB signalling in various proliferative disorders and autoimmune diseases.

### SUMMARY OF THE INVENTION

Provided herein are compound of formula (I) or a pharmaceutically acceptable salt or a stereoisomer thereof and pharmaceutical compositions comprising a compound of formula (I) or a pharmaceutically acceptable salt or a stereoisomer thereof that are useful as MALT-1 inhibitors and for the treatment of diseases or disorders dependent on or mediated by MALT-1. The present invention also provides a preparation of a compound of formula (I) or a pharmaceutically acceptable salt or a stereoisomer thereof.

In one aspect, the present invention provides a compound of formula (I):

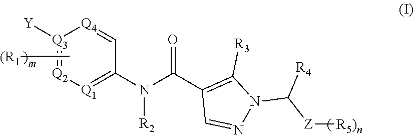

or a pharmaceutically acceptable salt or a stereoisomer thereof;
wherein;

each Q₁, Q₂, Q₃, and Q₄ independently represents N, CH or C, wherein at least one of Q₁, Q₂, Q₃ and Q₄ is N;

Y is absent or 5- to 6-membered heteroaryl;

R₁, at each occurrence, is hydroxy, hydroxyalkyl, halogen, alkoxy, alkyl, haloalkyl, haloalkoxy, amino, alkylamino, cyano or —CO—(5- to 6-membered heterocycloalkyl); wherein the said alkyl and haloalkyl, at each occurrence, are optionally substituted with 1 or 2 substituents independently selected from hydroxy, alkoxy and amino;

R₂ is hydrogen, halogen or alkyl;

Z represents phenyl or 5- to 6-membered heteroaryl;

R₃ is alkyl, haloalkyl, 3- to 10-membered cycloalkyl, 6- to 10-membered aryl or 5- to 12-membered heteroaryl;

R₄ is hydrogen, alkyl, haloalkyl or 3- to 10-membered cycloalkyl;

R₅, at each occurrence, is hydrogen, hydroxy, hydroxyalkyl, halogen, alkoxy, alkyl, haloalkyl, haloalkoxy, amino, alkylamino or cyano; or any two R₅ groups attached to adjacent carbon atoms of ring Z combine together to form Z₁ ring;

Z₁ represents fused benzo, fused 5- to 6-membered heteroaryl or fused 5- to 6-membered heterocycloalkyl ring;

m is 0, 1, 2 or 3, and

n is 1, 2 or 3.

In yet another aspect, the present invention provides a pharmaceutical composition comprising a compound of formula (I) or a pharmaceutically acceptable salt or a stereoisomer thereof and at least one pharmaceutically acceptable excipient (such as a pharmaceutically acceptable carrier or diluent).

In yet another aspect, the present invention relates to the preparation of a compound of formula (I) or a pharmaceutically acceptable salt or a stereoisomer thereof.

In another aspect, the present invention provides a pharmaceutical composition comprising a compound of formula (I) or a pharmaceutically acceptable salt or a stereoisomer thereof for use as a medicament.

In another aspect, the present invention provides a pharmaceutical composition comprising a compound of formula (I) or a pharmaceutically acceptable salt or a stereoisomer thereof for treating diseases or disorders that are dependent upon or mediated by MALT-1.

In another aspect, the present invention provides a pharmaceutical composition comprising a compound of formula (I) or a pharmaceutically acceptable salt or a stereoisomer thereof for treating diseases or disorders that have altered MALT-1 including mutations and overexpression thereof.

In another aspect, the present invention provides a pharmaceutical composition comprising a compound of formula (I) or a pharmaceutically acceptable salt or a stereoisomer thereof for treating diseases or conditions wherein inhibition of MALT-1 proteins provides a benefit.

In another aspect, the present invention provides a method of treating a disease or disorder mediated by the inhibition of MALT-1, in a subject comprising administering to the subject in need thereof a therapeutically effective amount of a compound of formula (I) or a pharmaceutically acceptable salt or a stereoisomer or a tautomer thereof. The disease or disorder dependent upon MALT-1 is cancer.

In another aspect, the present invention provides a use of a compound of formula (I) or a pharmaceutically acceptable salt or a stereoisomer thereof for the manufacture of a medicament for treating a disease or condition, e.g., cancer.

### DETAILED DESCRIPTION OF THE INVENTION

The present invention provides pyrazolyl derivative compounds, referred as a compound of formula (I), which are useful as MALT-1 inhibitors and for the treatment of conditions dependent on or mediated by MALT-1. The present invention further provides pharmaceutical compositions comprising the said compound or a stereoisomer thereof as therapeutic agents.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present invention includes such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present invention.

In one embodiment, the present invention provides compound of formula (I):

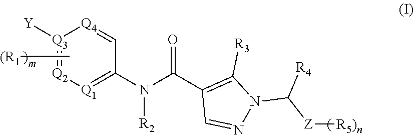

or a pharmaceutically acceptable salt or a stereoisomer thereof;
wherein;

each Q₁, Q₂, Q₃, and Q₄ independently represents N, CH or C, wherein at least one of Q₁, Q₂, Q₃ and Q₄ is N;

Y is absent or 5- to 6-membered heteroaryl;

R₁, at each occurrence, is hydroxy, hydroxyalkyl, halogen, alkoxy, alkyl, haloalkyl, haloalkoxy, amino, alkylamino, cyano or —CO—(5- to 6-membered heterocycloalkyl); wherein the said alkyl and haloalkyl, at each occurrence, are optionally substituted with 1 or 2 substituents independently selected from hydroxy, alkoxy and amino;

R₂ is hydrogen, halogen or alkyl;

Z represents phenyl or 5- to 6-membered heteroaryl;

R₃ is alkyl, haloalkyl, 3- to 10-membered cycloalkyl, 6- to 10-membered aryl or 5- to 12-membered heteroaryl;

R₄ is hydrogen, alkyl, haloalkyl or 3- to 10-membered cycloalkyl;

R₅, at each occurrence, is hydrogen, hydroxy, hydroxyalkyl, halogen, alkoxy, alkyl, haloalkyl, haloalkoxy, amino, alkylamino or cyano; or any two R₅ groups attached to adjacent carbon atoms of ring Z combine together to form Z₁ ring;

Z₁ represents fused benzo, fused 5- to 6-membered heteroaryl or fused 5- to 6-membered heterocycloalkyl ring;

m is 0, 1, 2 or 3, and

n is 1, 2 or 3.

In one embodiment, present invention provides compound of formula (I) wherein

i. Q₁ represents N; and Q₂, Q₃, and Q₄ independently represents C or CH;

ii. Q₂ represents N; and Q₁, Q₃, and Q₄ independently represents C or CH;

iii. Q₃ represents N; and Q₁, Q₂, and Q₄ independently represents C or CH;

iv. Q₂ and Q₄ independently represents N; and Q₁ and Q₃ independently represents C or CH;

v. Q₁ and Q₃ independently represents N; and Q₂ and Q₄ independently represents C or CH; or

vi. Q₁ and Q₂ independently represents N; and Q₃ and Q₄ independently represents C or CH.

In one embodiment, Q₁ represents N; and Q₂, Q₃, and Q₄ independently represents C or CH. In one embodiment, Q₂ represents N; and Q₁, Q₃, and Q₄ independently represents C or CH. In one embodiment, Q₃ represents N; and Q₁, Q₂, and Q₄ independently represents C or CH.

In one embodiment, Y is absent.

In one embodiment, Y represents thienyl, furanyl, pyrrolyl, imidazolyl, pyrazolyl, isoxazolyl, oxazolyl, isothiazolyl, thiazolyl, 1H-tetrazolyl, triazolyl, triazol-2-yl, oxadiazolyl, pyridazinyl pyridyl, pyrimidinyl or pyrazinyl. In one embodiment, Y represents thienyl, furanyl, pyrrolyl, imidazolyl, pyrazolyl, isoxazolyl, oxazolyl, isothiazolyl, thiazolyl, 1H-tetrazolyl, triazolyl, triazol-2-yl, oxadiazolyl, pyridazinyl, pyridyl, pyrimidinyl or pyrazinyl. In one embodiment, Y is 1,2,4-triazol-4-yl, 1,2,4-triazol-3-yl, 1,2,4-triazol-2-yl or 1,2,3-triazol-4-yl, 1,2,3-triazol-2-yl. In one embodiment, Y is 1,2,3-triazol-2-yl.

In one embodiment, R₁, at each occurrence, is hydroxy, hydroxyalkyl, halogen, alkoxy, alkyl, haloalkyl, haloalkoxy, amino, cyano or —CO—(5- to 6-membered heterocycloalkyl); wherein the said alkyl, at each occurrence, is optionally substituted with 1 or 2 substituents independently selected from oxo, hydroxy, alkoxy and amino.

In one embodiment, R₁, at each occurrence, is hydroxy, hydroxyalkyl, halogen, alkoxy, cyano, pyrrolidinyl-CO— or haloalkyl optionally substituted with —OH.

In one embodiment, R₁, at each occurrence, is —CN, —Cl, —CF₃, —O—CH₃, —F, pyrrolidinyl-CO— or —CH(OH)CF₃.

In one embodiment, R₁, at each occurrence, is —CN, —Cl, —CF₃, —O—CH₃, —F, pyrrolidinyl-CO— or —CH(OH)CF₃.

In one embodiment, R₁, at each occurrence, is —CN, —Cl, —CF₃, —O—CH₃ or —F.

In one embodiment, R₂ is hydrogen, halogen or alkyl. In one embodiment, R₂ is hydrogen.

In one embodiment, R₃ is alkyl, haloalkyl, 3- to 10-membered cycloalkyl, 6- to 10-membered aryl or 5- to 12-membered heteroaryl. In one embodiment, R₃ is haloalkyl or 3- to 10-membered cycloalkyl. In one embodiment, R₃ is —CF₃ or cyclopropyl. In one embodiment, R₃ is —CF₃. In one embodiment, R₃ is cyclopropyl.

In one embodiment, R₄ is hydrogen, alkyl or 3- to 10-membered cycloalkyl. In one embodiment, R₄ is hydrogen or (C₁-C₆-alkyl). In one embodiment, R₄ is hydrogen or —CH₃, —CH₂CH₃ or —CH(CH₃)₂.

In one embodiment, Z represents phenyl or 5- to 6-membered heteroaryl.

In one embodiment, Z represents phenyl, furanyl, thienyl, pyrrolyl, pyrazolyl, imidazolyl, oxazolyl, isoxazolyl, thiazolyl, isothiazolyl, 1H-tetrazolyl, oxadiazolyl, triazolyl, pyridyl, pyrimidinyl, pyrazinyl or pyridazinyl.

In one embodiment, Z represents phenyl or pyridyl.

In one embodiment, R₅, at each occurrence, is hydrogen, hydroxy, hydroxyalkyl, halogen, alkoxy, alkyl, haloalkyl, haloalkoxy, amino, alkylamino or cyano. In one embodiment, R₅, at each occurrence, is —Cl, —F, —Br, —CN, —CF₃, —OCH₃ or —CH₃.

In one embodiment, any two R₅ groups attached to adjacent carbon atoms combine together to form Z₁ ring.

In one embodiment, Z₁ represents fused benzo, fused 5- to 6-membered heteroaryl or fused 5- to 6-membered heterocycloalkyl ring.

In one embodiment, Z₁ represents

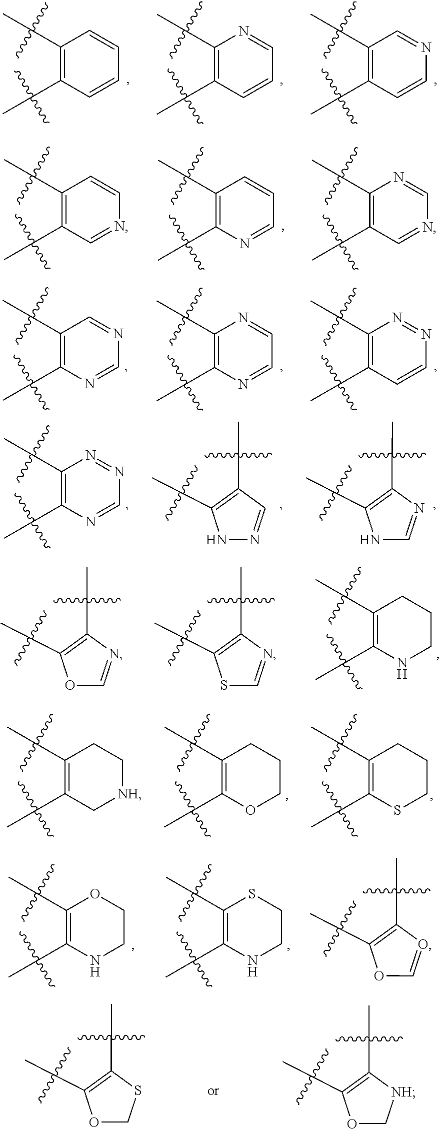

wherein [symbol] represents the points of fusion with Z.

In one embodiment, Z₁ represents

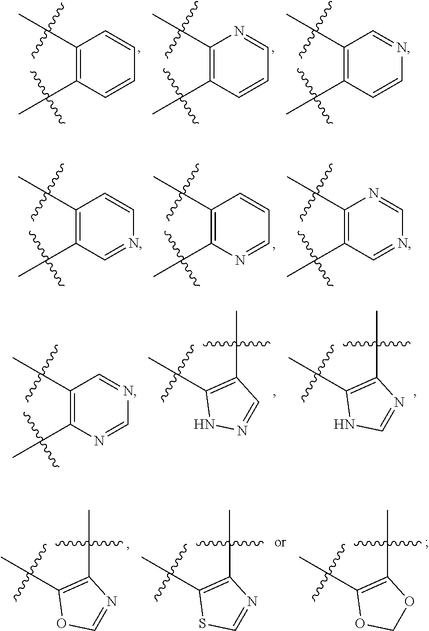

wherein [symbol] represents the points of fusion with Z.

In one embodiment, m is 0, 1, 2 or 3. In one embodiment, m is 1, 2 or 3.

In one embodiment, n is 1, 2 or 3.

In one embodiment, the present invention provides compound of formula (I) or a pharmaceutically acceptable salt or a stereoisomer thereof:

each Q₁, Q₂, Q₃, and Q₄ independently represents N, CH or C, wherein at least one of Q₁, Q₂, Q₃ and Q₄ are N;

Y is absent or 5- to 6-membered heteroaryl;

R₁, at each occurrence, is hydroxy, hydroxyalkyl, halogen, alkoxy, —CN, pyrrolidinyl-CO— or haloalkyl optionally substituted with —OH;

R₂ is hydrogen;

Z represents phenyl, furanyl, thienyl, pyrrolyl, pyrazolyl, imidazolyl, oxazolyl, isoxazolyl, thiazolyl, isothiazolyl, 1H-tetrazolyl, oxadiazolyl, triazolyl, pyridyl, pyrimidinyl, pyrazinyl or pyridazinyl;

R₃ is alkyl, haloalkyl or cyclopropyl;

R₄ is hydrogen or (C₁-C₆-alkyl);

R₅, at each occurrence, is hydrogen, halogen, alkoxy, alkyl, haloalkyl or cyano; or any two R₅ groups attached to adjacent carbon atoms combine together to form Z₁ ring;

Z₁ represents

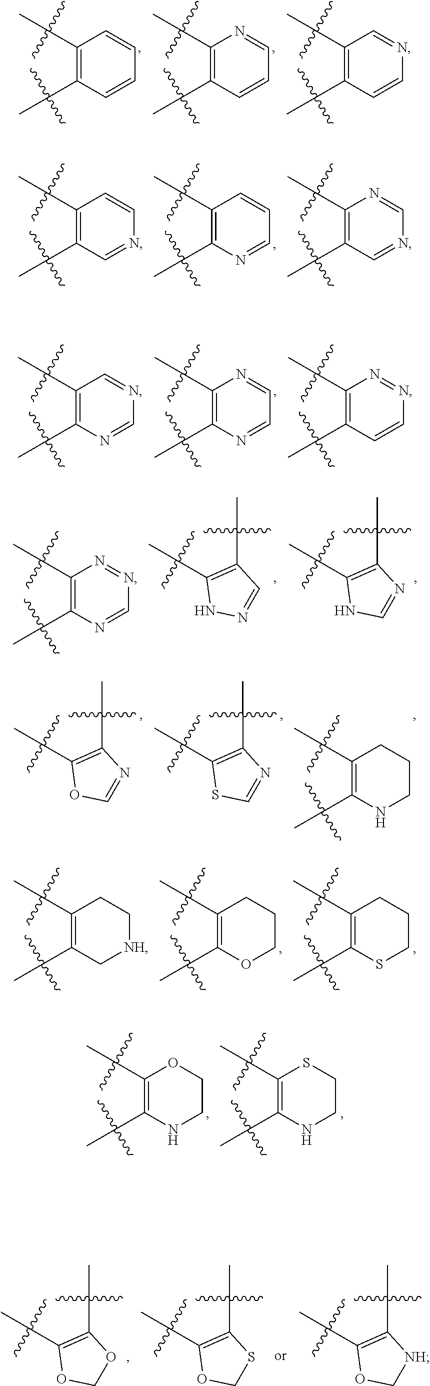

wherein [symbol] represents the points of fusion with Z;

m is 0, 1, 2 or 3; and

n is 1, 2 or 3.

In one embodiment, the present invention provides compound of formula (I) or a pharmaceutically acceptable salt or a stereoisomer thereof:

Q₁, Q₂, Q₃ and Q₄ are selected from (i), (ii), (iii), (iv), (v) and (vi):

i. Q₁ represents N; and Q₂, Q₃, and Q₄ independently represents C or CH;

ii. Q₂ represents N; and Q₁, Q₃, and Q₄ independently represents C or CH;

iii. Q₃ represents N; and Q₁, Q₂, and Q₄ independently represents C or CH;

iv. Q₂ and Q₄ independently represents N; and Q₁ and Q₃ independently represents C or CH;

v. Q₁ and Q₃ independently represents N; and Q₂ and Q₄ independently represents C or CH;

vi. Q₁ and Q₂ independently represents N; and Q₃ and Q₄ independently represents C or CH.

R₁, at each occurrence, is —CN, —Cl, —CF₃, —F, pyrrolidinyl-CO— or —CH(OH)CF₃;

R₂ is hydrogen;

Z represents phenyl or pyridyl;

R₃ is —CF₃ or cyclopropyl;

R₄ is hydrogen or —CH₃, —CH₂CH₃ or —CH(CH₃)₂;

R₅, at each occurrence, is —Cl, —F, —Br, —CN, —CF₃, —OCH₃ or —CH₃; or any two R₅ groups attached to adjacent carbon atoms combine together to form Z₁ ring;

Z₁ represents

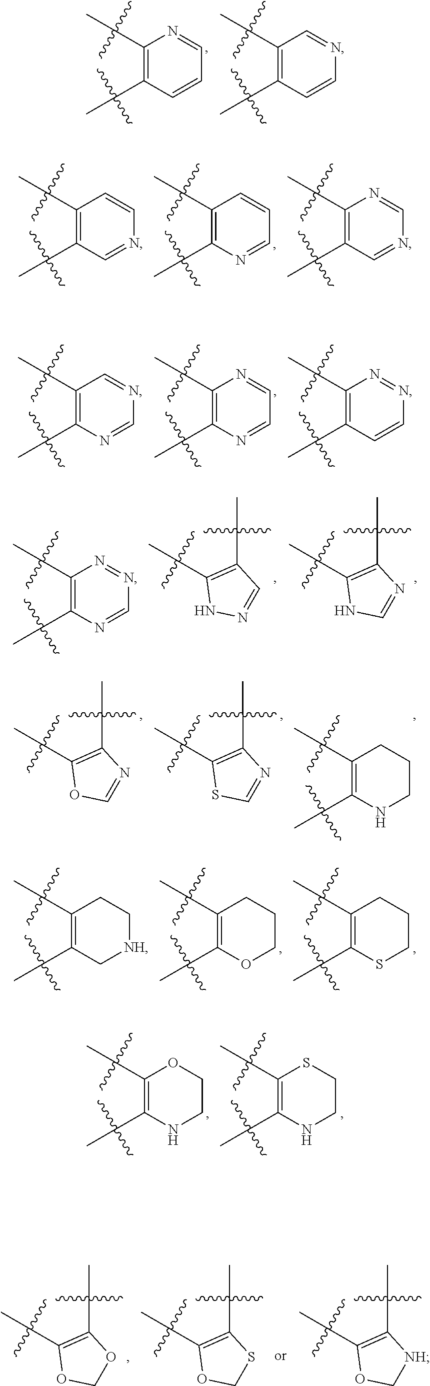

[symbol]

represents the points of fusion with Z;

m is 0, 1, 2 or 3; and

n is 1, 2 or 3.

In one embodiment, the present invention provides compound of formula (IA) or a pharmaceutically acceptable salt or a stereoisomer thereof:

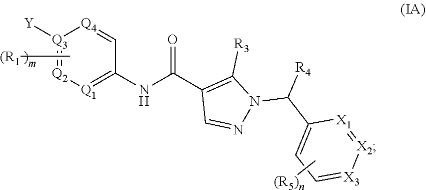

wherein each X₁, X₂ and X₃ independently represents N or C.

In one embodiment of compound of formula (IA), —X₁—X₂—X₃—represents —C—C—C—, —N—C—C—, —C—N—C— or —C—C—N.

In one embodiment of compound of formula (IA), —X₁—X₂—X₃—represents —N—C—C—, —C—N—C— or —C—C—N. In one embodiment of compound of formula (IA), —X₁—X₂—X₃—represents —N—C—C—. In one embodiment of compound of formula (IA), —X₁—X₂—X₃—represents —C—N—C—. In one embodiment of compound of formula (IA), —X₁—X₂—X₃—represents —C—C—N—.

In one embodiment of compound of formula (IA), R₁, at each occurrence, is halogen, alkoxy, cyano or haloalkyl optionally substituted with —OH.

In one embodiment of compound of formula (IA), R₁, at each occurrence, is —CN, —Cl, —CF₃, —O—CH₃, —F, pyrrolidinyl-CO— or —CH(OH)CF₃. In one embodiment of compound of formula (IA), R₁, at each occurrence, is —CN, —Cl, —CF₃, —O—CH₃, —F, pyrrolidinyl-CO— or —CH(OH)CF₃.

In one embodiment of compound of formula (IA), R₃ is haloalkyl or cycloalkyl. In one embodiment of compound of formula (IA), R₃ is —CF₃ or cyclopropyl.

In one embodiment of compound of formula (IA), R₄ is hydrogen or (C₁-C₆) alkyl. In one embodiment of compound of formula (IA), R₄ is hydrogen, —CH₃, —CH₂CH₃ or —CH(CH₃)₂.

In one embodiment of compound of formula (IA), R₅, at each occurrence, is hydrogen, halogen, haloalkyl, alkyl, cyano, hydroxy or alkoxy. In one embodiment of compound of formula (IA), R₅, at each occurrence, is —Cl, —F, —Br, —CN, —CF₃, —OCH₃ or —CH₃. In one embodiment of compound of formula (IA), any two R₅ groups attached to adjacent carbon atoms combine together to form Z₁ ring.

In one embodiment of compound of formula (IA), Z₁ represents fused benzo, fused 5- to 6-membered heteroaryl or fused 5- to 6-membered heterocycloalkyl ring.

In one embodiment of compound of formula (IA), Z₁ represents

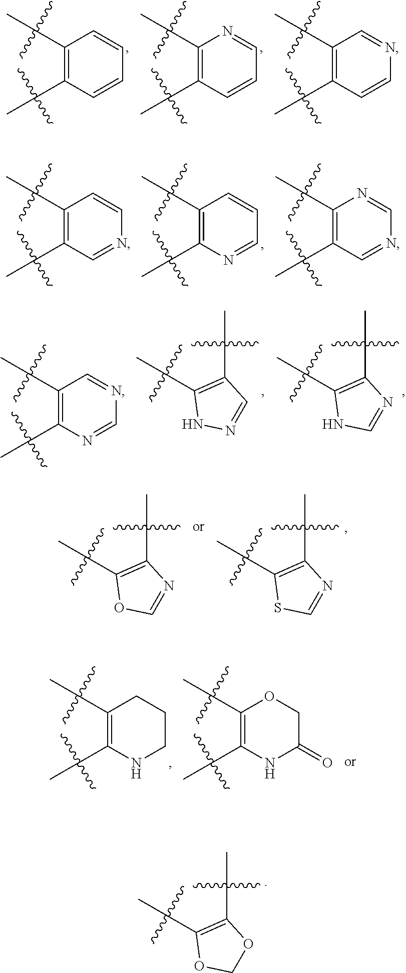

In one embodiment of compound of formula (IA), Z₁ represents

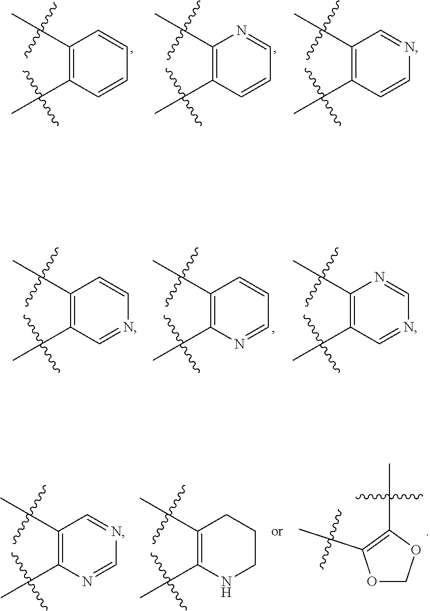

In one embodiment, the present invention provides a compound of formula (IA) or a pharmaceutically acceptable salt or a stereoisomer thereof: wherein,

X₁—X₂—X₃— represents —C—C—C—, —N—C—C, —C—N—C or —C—C—N—;

each Q₁, Q₂, Q₃, and Q₄ independently represents N, CH or C, wherein at least one of Q₁, Q₂, Q₃ and Q₄ are N;

R₁, at each occurrence, is halogen, alkoxy, cyano, —CO—(5 to 6-membered heterocycloalkyl), haloalkyl optionally substituted with —OH;

R₃ is haloalkyl or cycloalkyl;

R₄ is hydrogen or alkyl;

R₅, at each occurrence, is hydrogen, halogen, haloalkyl, alkyl, cyano, hydroxy or alkoxy; or any two R₅ groups attached to adjacent carbon atoms combine together to form Z₁ ring;

Z₁ represents fused benzo, fused 5- to 6-membered heteroaryl or fused 5- to 6-membered heterocycloalkyl ring.

In one embodiment, the present invention provides a compound of formula (IA), wherein,

—X₁—X₂—X₃— represents —C—C—C—, —N—C—C, —C—N—C or —C—C—N—;

each Q₁, Q₂, Q₃, and Q₄ independently represents N, CH or C, wherein at least one of Q₁, Q₂, Q₃ and Q₄ are N;

R₁, at each occurrence, is —CN, —Cl, —CF₃, —O—CH₃, —F, pyrrolidinyl-CO— or —CH(OH)CF₃;

R₃ is —CF₃ or cyclopropyl;

R₄ is hydrogen, —CH₃, —CH₂CH₃ or —CH(CH₃)₂;

R₅, at each occurrence, is —Cl, —F, —Br, —CN, —CF₃, —OCH₃ or —CH₃; or any two R₅ groups attached to adjacent carbon atoms combine together to form Z₁ ring;

Z₁ represents

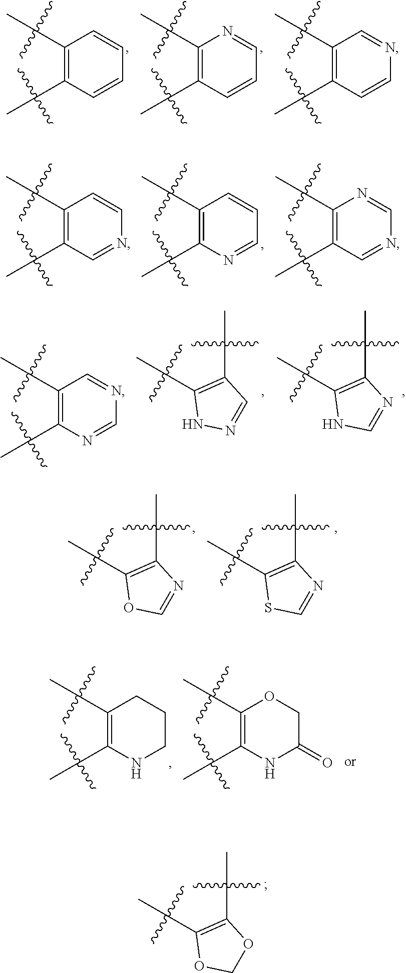

m is 0, 1, 2 or 3, and

n is 1, 2 or 3.

In one embodiment, the present invention provides compound of formula (IB) or a pharmaceutically acceptable salt or a stereoisomer thereof:

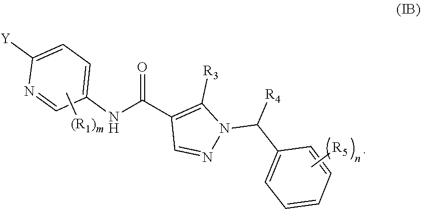

In one embodiment of compound of formula (IB), R₁, each occurrence, is halogen, alkoxy, haloalkyl, —CN or pyrrolidinyl-CO—.

In one embodiment of compound of formula (IB), R₃ is haloalkyl or cycloalkyl. In one embodiment of compound of formula (IB), R₃ is —CF₃ or cyclopropyl.

In one embodiment of compound of formula (IB), R₄ is hydrogen or alkyl. In one embodiment of compound of formula (IB), R₄ is hydrogen, —CH₃, —CH₂CH₃ or —CH(CH₃)₂.

In one embodiment of compound of formula (IB), R₅, each occurrence, is hydrogen, halogen, haloalkyl, alkyl, cyano, hydroxy or alkoxy; or any two R₅ groups attached to adjacent carbon atoms combine together to form Z₁ ring. In one embodiment of compound of formula (IB), R₅, at each occurrence, is —Cl, —F, —Br, —CN, —CF₃, —OCH₃ or —CH₃.

In one embodiment of compound of formula (IB), Z₁ represents fused benzo, fused 5- to 6-membered heteroaryl or fused 5- to 6-membered heterocycloalkyl ring. In one embodiment of compound of formula (IB), Z₁ represents

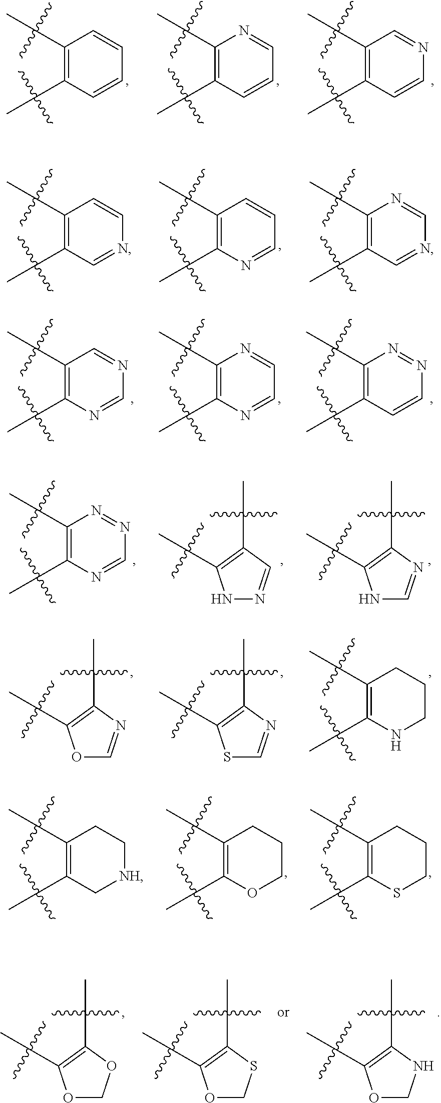

In one embodiment of compound of formula (TB), Z₁ represents

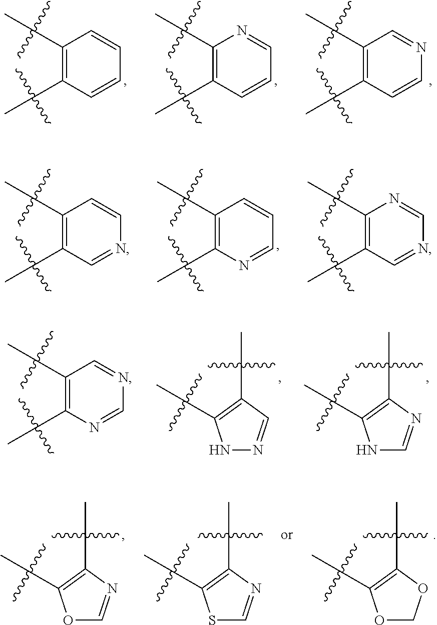

In one embodiment, the present invention provides compound of formula (IB) or a pharmaceutically acceptable salt or a stereoisomer thereof: wherein

R₁ at each occurrence, is halogen, alkoxy, haloalkyl, —CN pyrrolidinyl-CO—;

R₃ is haloalkyl or cycloalkyl;

R₄ is hydrogen or alkyl;

R₅, at each occurrence, is hydrogen, halogen, haloalkyl, alkyl, cyano, hydroxy or alkoxy; or any two R₅ groups attached to adjacent carbon atoms combine together to form Z₁ ring;

Z₁ represents fused benzo, fused 5- to 6-membered heteroaryl or fused 5- to 6-membered heterocycloalkyl ring.

In one embodiment, the present invention provides compound of formula (IB) or a pharmaceutically acceptable salt or a stereoisomer thereof: wherein

R₁, at each occurrence, is —CN, —Cl, —CF₃, —O—CH₃, —F, or pyrrolidinyl-CO—;

R₃ is —CF₃ or cyclopropyl;

R₄ is hydrogen, —CH₃, —CH₂CH₃ or —CH(CH₃)₂;

R₅, at each occurrence, is —Cl, —F, —Br, —CN, —CF₃, —OCH₃ or —CH₃; or any two R₅ groups attached to adjacent carbon atoms combine together to form Z₁ ring;

Z₁ represents

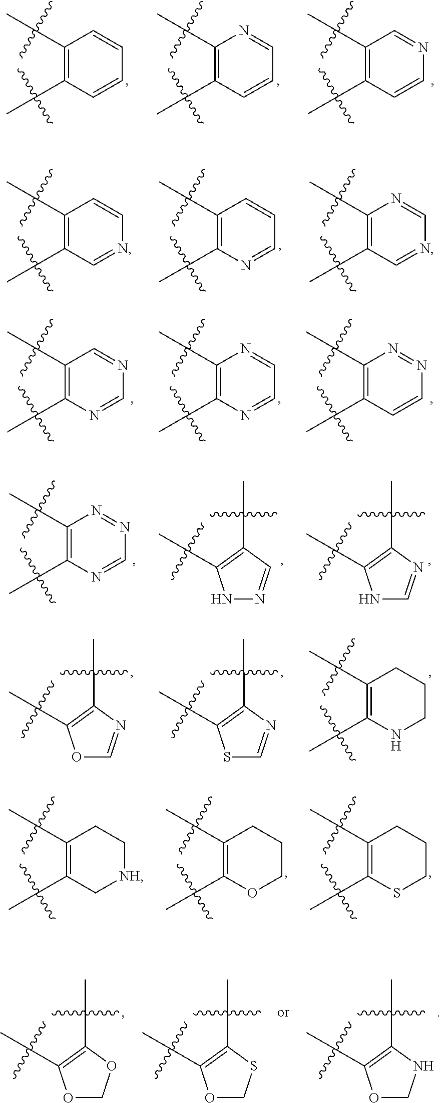

In one embodiment, the present invention provides compound of formula (IC) or a pharmaceutically acceptable salt or a stereoisomer thereof:

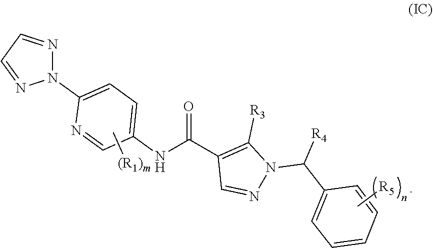

In one embodiment of compound of formula (IC), R₁, at each occurrence, is halogen, haloalkyl or —CN;

In one embodiment of formula (IC), R₁, at each occurrence, is —CN, —Cl, —CF₃, —O—CH₃ or —F.

In one embodiment of compound of formula (IC), R₃ is —CF₃ or cyclopropyl.

In one embodiment of compound of formula (IC), R₄ is hydrogen or (C₁-C₆)alkyl. In one embodiment of compound of formula (IC), R₄ is hydrogen, —CH₃, —CH₂CH₃ or —CH(CH₃)₂.

In one embodiment of compound of formula (IC), R₅, at each occurrence, is hydrogen, halogen, haloalkyl, alkyl, cyano, hydroxy or alkoxy. In one embodiment of compound of formula (IC), R₅, at each occurrence, is —Cl, —F, —Br, —CN, —CF₃, —OCH₃ or —CH₃.

In one embodiment of compound of formula (IC), Z₁ represents

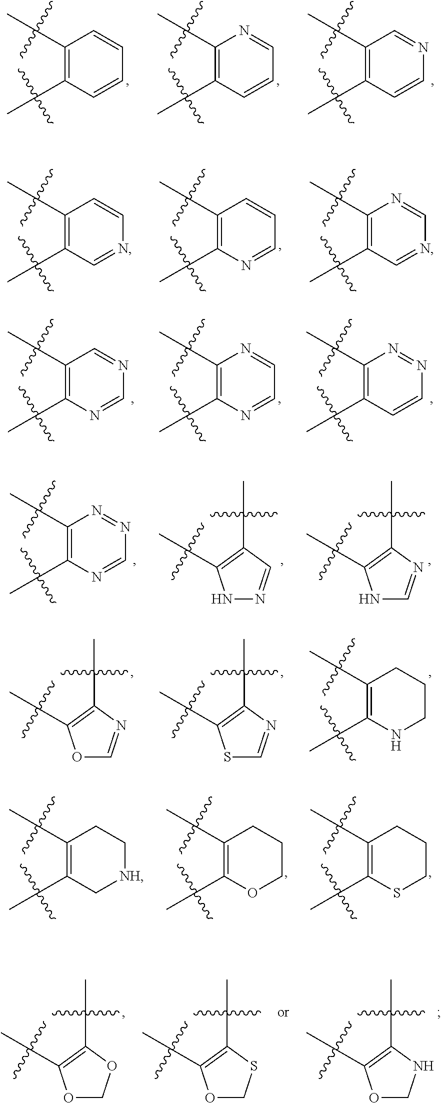

wherein [symbol] represents the points of fusion with Z.

In one embodiment of compound of formula (IC), Z₁ represents

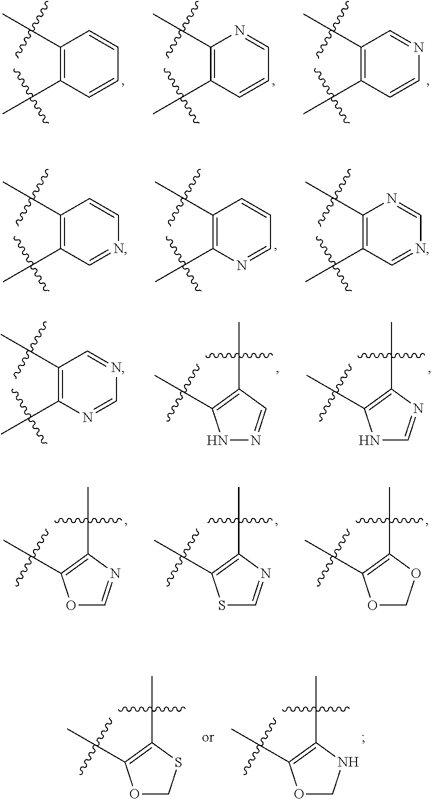

wherein [symbol] represents the points of fusion with Z.

In one embodiment of compound of formula (IC), m is 1, 2 or 3. In one embodiment of compound of formula (IC), n is 1, 2 or 3.

In one embodiment, the present invention provides compound of formula (IC) or a pharmaceutically acceptable salt or a stereoisomer thereof: wherein

R₁, at each occurrence, is —CN, —Cl, —CF₃, —O—CH₃ or —F;

R₃ is —CF₃ or cyclopropyl;

R₄ is hydrogen, —CH₃, —CH₂CH₃ or —CH(CH₃)₂;

R₅, at each occurrence, is —Cl, —F, —Br, —CN, —CF₃, —OCH₃ or —CH₃; or any two R₅ groups attached to adjacent carbon atoms combine together to form Z₁ ring;

Z₁ represents

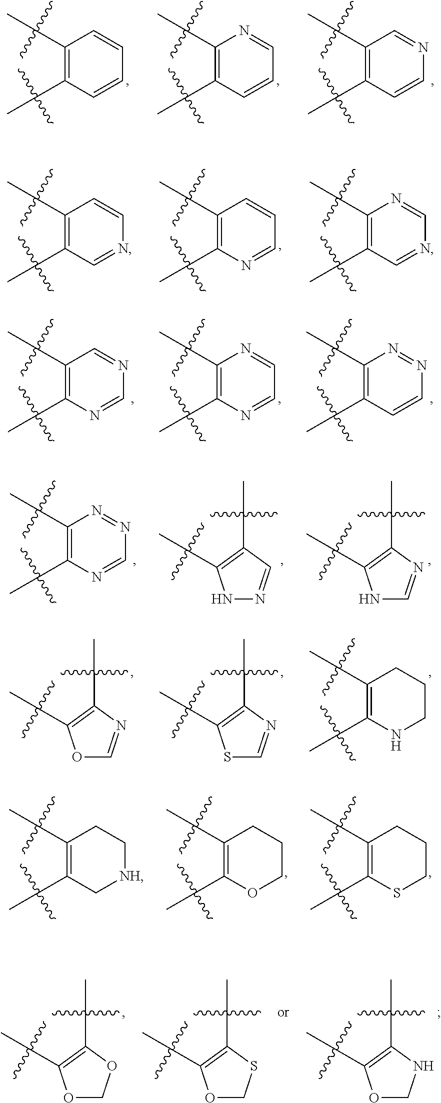

wherein [symbol] represents the points of fusion with Z;

m is 1, 2 or 3, and

n is 1, 2 or 3.

In one embodiment, the present invention provides a compound selected from:

| Compound | Structure |
|---|---|
| 1 | 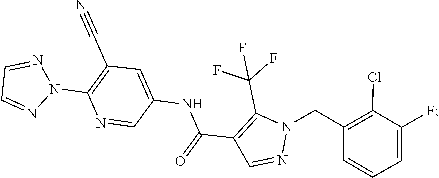 [SMILES: N#Cc1cc(NC(=O)c2cnn(Cc3cccc(F)c3Cl)c2C(F)(F)F)cnc1-n1nccn1] |
| 2 | 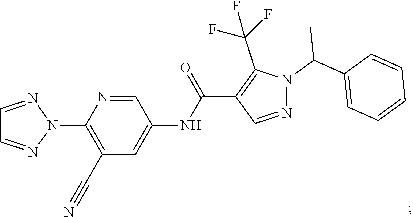 [SMILES: CC(c1ccccc1)n1ncc(C(=O)Nc2cnc(-n3nccn3)c(C#N)c2)c1C(F)(F)F] |
| 2a | 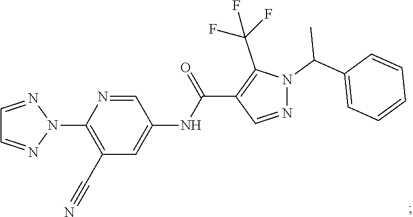 [SMILES: CC(c1ccccc1)n1ncc(C(=O)Nc2cnc(-n3nccn3)c(C#N)c2)c1C(F)(F)F] |
| 2b | 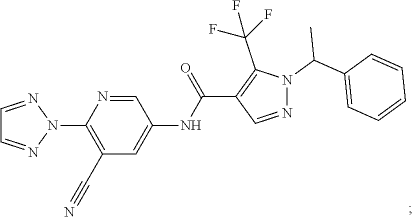 [SMILES: CC(c1ccccc1)n1ncc(C(=O)Nc2cnc(-n3nccn3)c(C#N)c2)c1C(F)(F)F] |
| 3 | 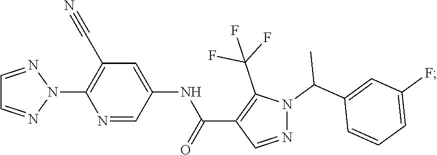 [SMILES: CC(c1cccc(F)c1)n1ncc(C(=O)Nc2cnc(-n3nccn3)c(C#N)c2)c1C(F)(F)F] |
| 3a | 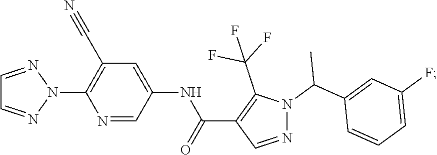 [SMILES: CC(c1cccc(F)c1)n1ncc(C(=O)Nc2cnc(-n3nccn3)c(C#N)c2)c1C(F)(F)F] |
| 3b | 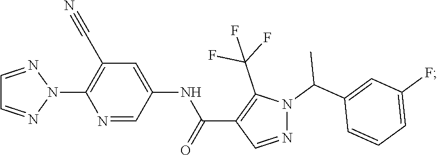 [SMILES: CC(c1cccc(F)c1)n1ncc(C(=O)Nc2cnc(-n3nccn3)c(C#N)c2)c1C(F)(F)F] |
| 4 | 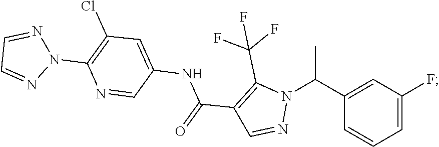 [SMILES: CC(c1cccc(F)c1)n1ncc(C(=O)Nc2cnc(-n3nccn3)c(Cl)c2)c1C(F)(F)F] |
| 4a | 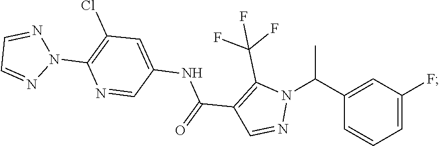 [SMILES: CC(c1cccc(F)c1)n1ncc(C(=O)Nc2cnc(-n3nccn3)c(Cl)c2)c1C(F)(F)F] |
| 4b | 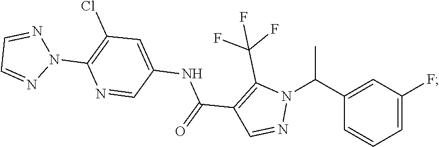 [SMILES: CC(c1cccc(F)c1)n1ncc(C(=O)Nc2cnc(-n3nccn3)c(Cl)c2)c1C(F)(F)F] |
| 5 | 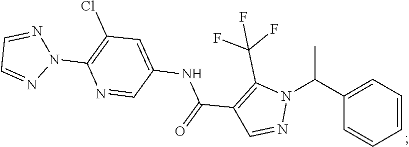 [SMILES: CC(c1ccccc1)n1ncc(C(=O)Nc2cnc(-n3nccn3)c(Cl)c2)c1C(F)(F)F] |
| 5a | 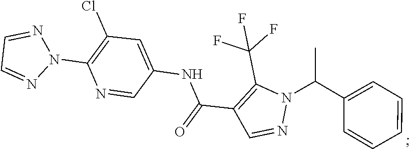 [SMILES: CC(c1ccccc1)n1ncc(C(=O)Nc2cnc(-n3nccn3)c(Cl)c2)c1C(F)(F)F] |
| 5b | 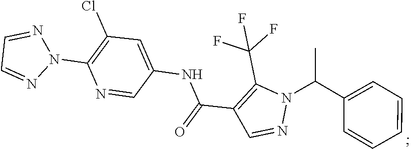 [SMILES: CC(c1ccccc1)n1ncc(C(=O)Nc2cnc(-n3nccn3)c(Cl)c2)c1C(F)(F)F] |
| 6 | 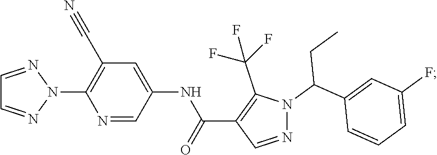 |
| 7 | 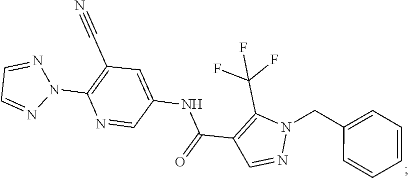 [SMILES: N#Cc1cc(NC(=O)c2cnn(Cc3ccccc3)c2C(F)(F)F)cnc1-n1nccn1] |
| 8 | 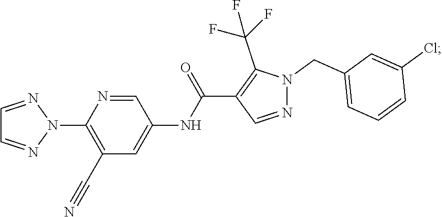 [SMILES: N#Cc1cc(NC(=O)c2cnn(Cc3cccc(Cl)c3)c2C(F)(F)F)cnc1-n1nccn1] |
| 9 | 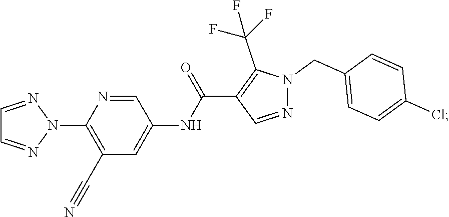 [SMILES: N#Cc1cc(NC(=O)c2cnn(Cc3ccc(Cl)cc3)c2C(F)(F)F)cnc1-n1nccn1] |
| 10 | 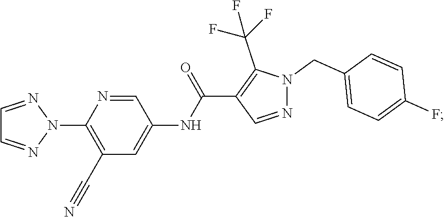 |
| 11 | 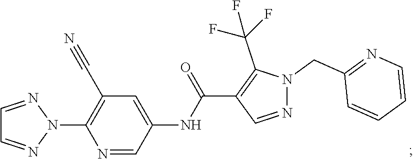 |
| 12 | 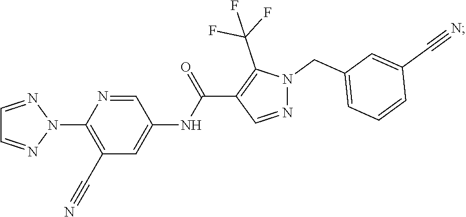 [SMILES: N#Cc1cccc(Cn2ncc(C(=O)Nc3cnc(-n4nccn4)c(C#N)c3)c2C(F)(F)F)c1] |
| 13 | 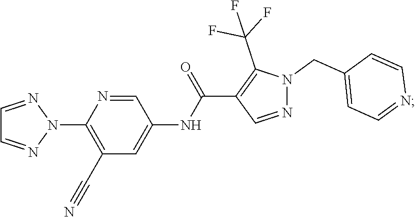 [SMILES: N#Cc1cc(NC(=O)c2cnn(Cc3ccncc3)c2C(F)(F)F)cnc1-n1nccn1] |
| 14 | 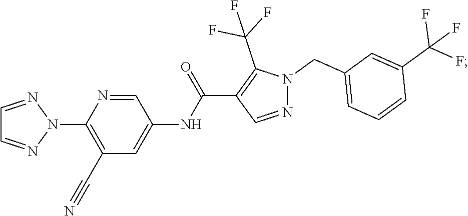 [SMILES: N#Cc1cc(NC(=O)c2cnn(Cc3cccc(C(F)(F)F)c3)c2C(F)(F)F)cnc1-n1nccn1] |
| 15 | 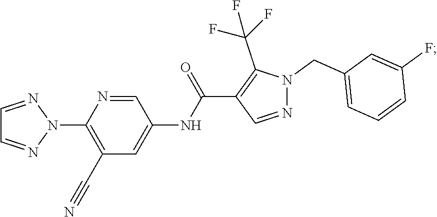 [SMILES: N#Cc1cc(NC(=O)c2cnn(Cc3cccc(F)c3)c2C(F)(F)F)cnc1-n1nccn1] |
| 16 | 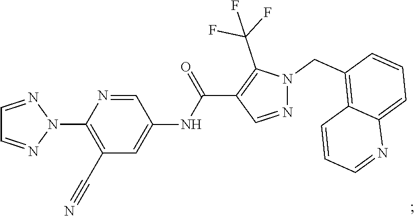 |
| 17 | 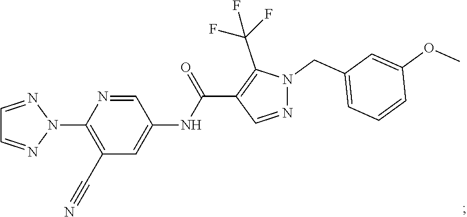 [SMILES: COc1cccc(Cn2ncc(C(=O)Nc3cnc(-n4nccn4)c(C#N)c3)c2C(F)(F)F)c1] |
| 18 | 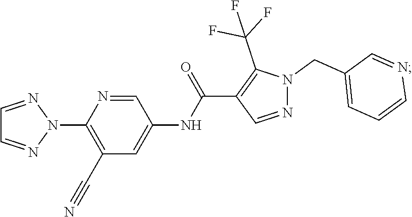 [SMILES: N#Cc1cc(NC(=O)c2cnn(Cc3cccnc3)c2C(F)(F)F)cnc1-n1nccn1] |
| 19 | 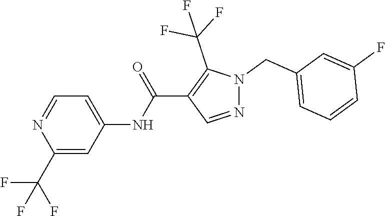 [SMILES: O=C(Nc1ccnc(C(F)(F)F)c1)c1cnn(Cc2cccc(F)c2)c1C(F)(F)F] |
| 20 | 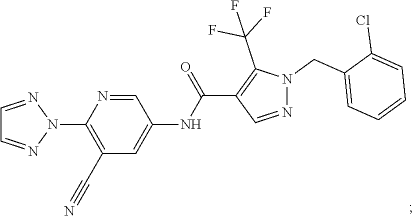 [SMILES: N#Cc1cc(NC(=O)c2cnn(Cc3ccccc3Cl)c2C(F)(F)F)cnc1-n1nccn1] |
| 21 | 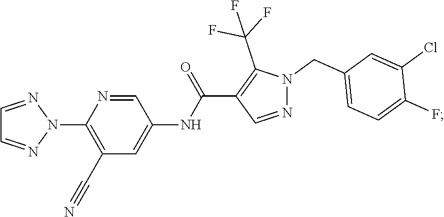 [SMILES: N#Cc1cc(NC(=O)c2cnn(Cc3ccc(F)c(Cl)c3)c2C(F)(F)F)cnc1-n1nccn1] |
| 22 | 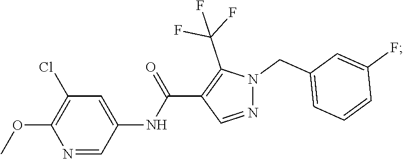 [SMILES: COc1ncc(NC(=O)c2cnn(Cc3cccc(F)c3)c2C(F)(F)F)cc1Cl] |
| 23 | 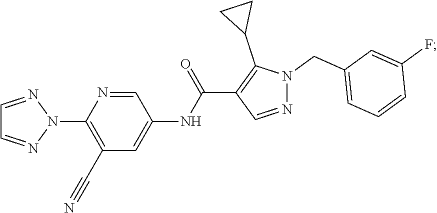 [SMILES: N#Cc1cc(NC(=O)c2cnn(Cc3cccc(F)c3)c2C2CC2)cnc1-n1nccn1] |
| 24 | 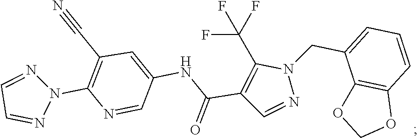 [SMILES: N#Cc1cc(NC(=O)c2cnn(Cc3cccc4c3OCO4)c2C(F)(F)F)cnc1-n1nccn1] |
| 25 | 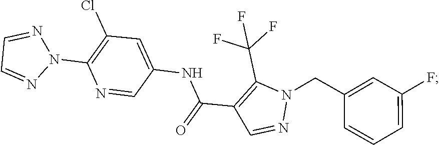 [SMILES: O=C(Nc1cnc(-n2nccn2)c(Cl)c1)c1cnn(Cc2cccc(F)c2)c1C(F)(F)F] |
| 26 | 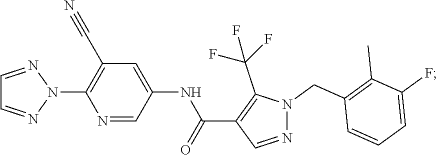 |
| 27 | 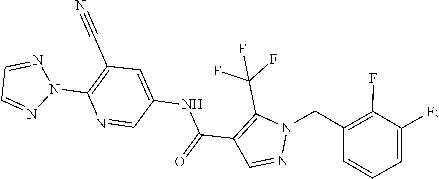 [SMILES: N#Cc1cc(NC(=O)c2cnn(Cc3cccc(F)c3F)c2C(F)(F)F)cnc1-n1nccn1] |
| 28 | 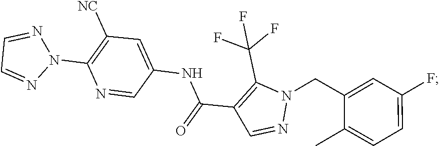 [SMILES: Cc1ccc(F)cc1Cn1ncc(C(=O)Nc2cnc(-n3nccn3)c(C#N)c2)c1C(F)(F)F] |
| 29 | 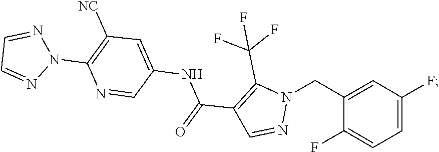 [SMILES: N#Cc1cc(NC(=O)c2cnn(Cc3cc(F)ccc3F)c2C(F)(F)F)cnc1-n1nccn1] |
| 30 | 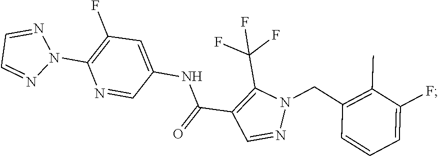 [SMILES: Cc1c(F)cccc1Cn1ncc(C(=O)Nc2cnc(-n3nccn3)c(F)c2)c1C(F)(F)F] |
| 31 | 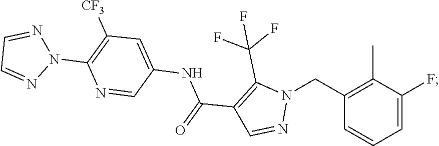 [SMILES: Cc1c(F)cccc1Cn1ncc(C(=O)Nc2cnc(-n3nccn3)c(C(F)(F)F)c2)c1C(F)(F)F] |
| 32 | 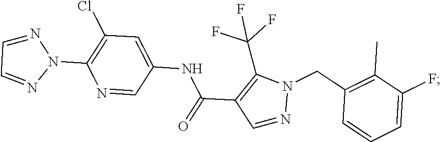 [SMILES: Cc1c(F)cccc1Cn1ncc(C(=O)Nc2cnc(-n3nccn3)c(Cl)c2)c1C(F)(F)F] |
| 33 | 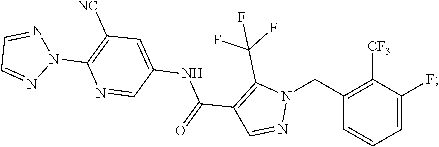 [SMILES: N#Cc1cc(NC(=O)c2cnn(Cc3cccc(F)c3C(F)(F)F)c2C(F)(F)F)cnc1-n1nccn1] |
| 34 | 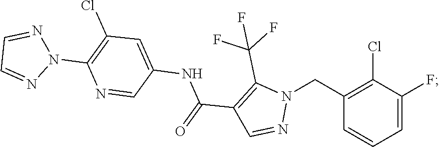 [SMILES: O=C(Nc1cnc(-n2nccn2)c(Cl)c1)c1cnn(Cc2cccc(F)c2Cl)c1C(F)(F)F] |
| 35 | 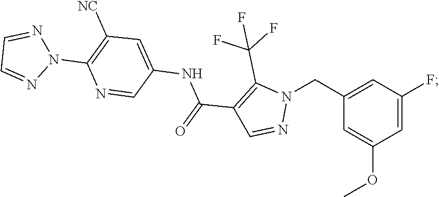 [SMILES: COc1cc(F)cc(Cn2ncc(C(=O)Nc3cnc(-n4nccn4)c(C#N)c3)c2C(F)(F)F)c1] |
| 36 | 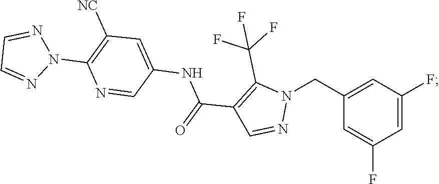 [SMILES: N#Cc1cc(NC(=O)c2cnn(Cc3cc(F)cc(F)c3)c2C(F)(F)F)cnc1-n1nccn1] |
| 37 | 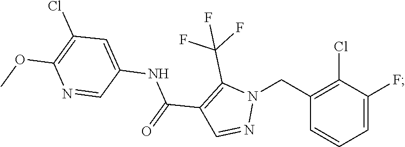 [SMILES: COc1ncc(NC(=O)c2cnn(Cc3cccc(F)c3Cl)c2C(F)(F)F)cc1Cl] |
| 38 | 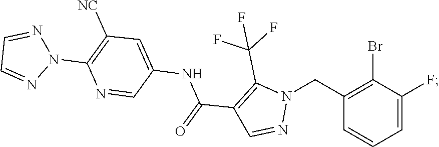 [SMILES: N#Cc1cc(NC(=O)c2cnn(Cc3cccc(F)c3Br)c2C(F)(F)F)cnc1-n1nccn1] |
| 39 | 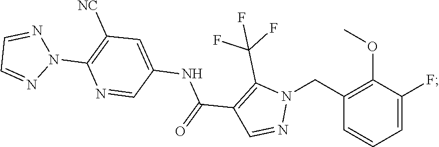 [SMILES: COc1c(F)cccc1Cn1ncc(C(=O)Nc2cnc(-n3nccn3)c(C#N)c2)c1C(F)(F)F] |
| 40 | 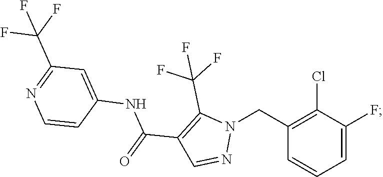 [SMILES: O=C(Nc1ccnc(C(F)(F)F)c1)c1cnn(Cc2cccc(F)c2Cl)c1C(F)(F)F] |
| 41 | 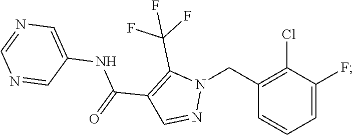 [SMILES: O=C(Nc1cncnc1)c1cnn(Cc2cccc(F)c2Cl)c1C(F)(F)F] |
| 42 | 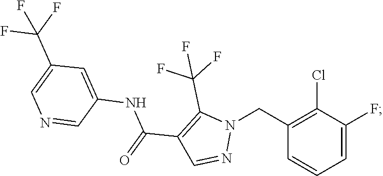 [SMILES: O=C(Nc1cncc(C(F)(F)F)c1)c1cnn(Cc2cccc(F)c2Cl)c1C(F)(F)F] |
| 43 | 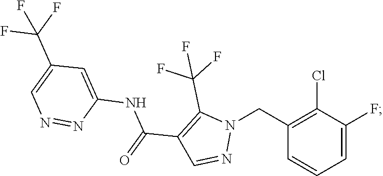 [SMILES: O=C(Nc1cc(C(F)(F)F)cnn1)c1cnn(Cc2cccc(F)c2Cl)c1C(F)(F)F] |
| 44 | 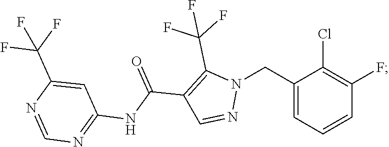 [SMILES: O=C(Nc1cc(C(F)(F)F)ncn1)c1cnn(Cc2cccc(F)c2Cl)c1C(F)(F)F] |
| 45 | 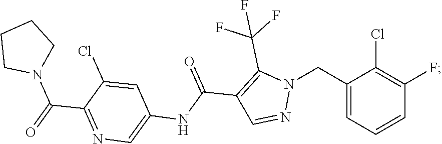 [SMILES: O=C(Nc1cnc(C(=O)N2CCCC2)c(Cl)c1)c1cnn(Cc2cccc(F)c2Cl)c1C(F)(F)F] |
| 46 | 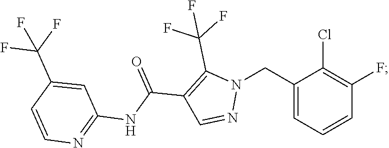 [SMILES: O=C(Nc1cc(C(F)(F)F)ccn1)c1cnn(Cc2cccc(F)c2Cl)c1C(F)(F)F] |
| 47 | 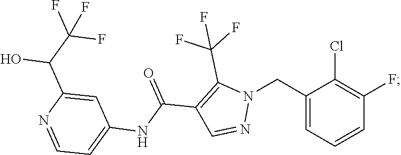 [SMILES: O=C(Nc1ccnc(C(O)C(F)(F)F)c1)c1cnn(Cc2cccc(F)c2Cl)c1C(F)(F)F] |
| 48 | 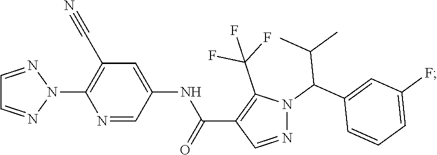 [SMILES: CC(C)C(c1cccc(F)c1)n1ncc(C(=O)Nc2cnc(-n3nccn3)c(C#N)c2)c1C(F)(F)F] |
| 49 | 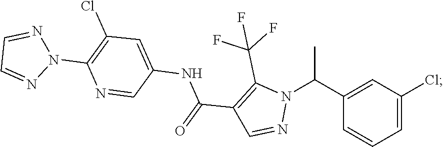 [SMILES: CC(c1cccc(Cl)c1)n1ncc(C(=O)Nc2cnc(-n3nccn3)c(Cl)c2)c1C(F)(F)F] |
| 50 | 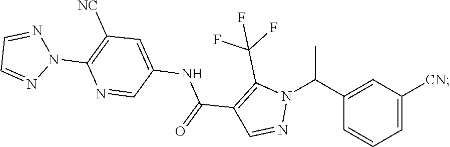 [SMILES: CC(c1cccc(C#N)c1)n1ncc(C(=O)Nc2cnc(-n3nccn3)c(C#N)c2)c1C(F)(F)F] |
| 51 | 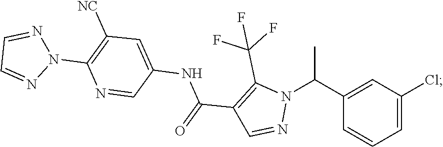 [SMILES: CC(c1cccc(Cl)c1)n1ncc(C(=O)Nc2cnc(-n3nccn3)c(C#N)c2)c1C(F)(F)F] |
| 52 | 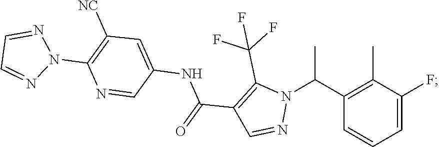 [SMILES: Cc1c(F)cccc1C(C)n1ncc(C(=O)Nc2cnc(-n3nccn3)c(C#N)c2)c1C(F)(F)F] |
| 53 | 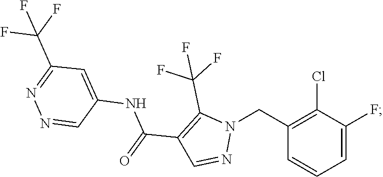 [SMILES: O=C(Nc1cnnc(C(F)(F)F)c1)c1cnn(Cc2cccc(F)c2Cl)c1C(F)(F)F] |
| 54 | 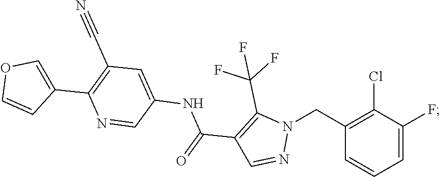 [SMILES: N#Cc1cc(NC(=O)c2cnn(Cc3cccc(F)c3Cl)c2C(F)(F)F)cnc1-c1ccoc1] |
| 55 | 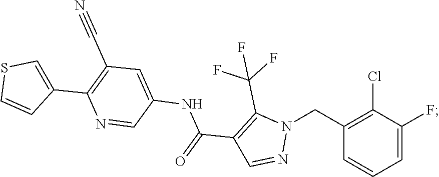 [SMILES: N#Cc1cc(NC(=O)c2cnn(Cc3cccc(F)c3Cl)c2C(F)(F)F)cnc1-c1ccsc1] |
| 56 | 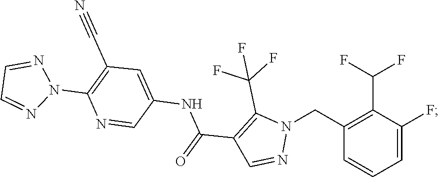 [SMILES: N#Cc1cc(NC(=O)c2cnn(Cc3cccc(F)c3C(F)F)c2C(F)(F)F)cnc1-n1nccn1] |

or a pharmaceutically acceptable salt or a stereoisomer or a tautomer thereof

### Method of Treatment

In one embodiment, the present invention provides a compound of formula (I) or a pharmaceutical acceptable salt or a stereoisomer or a tautomer thereof, for use as a medicament.

In one embodiment, the present invention provides a compound of formula (I) or a pharmaceutical acceptable salt or a stereoisomer or a tautomer thereof, for use in treating a disease or disorder mediated by MALT-1.

In one embodiment, the present invention provides a pharmaceutical composition comprising compound of formula (I) or a pharmaceutical acceptable salt or a stereoisomer or a tautomer thereof, for use in the manufacture of medicament for the treatment of disease or disorder mediated by MALT-1.

In certain embodiment, the present invention provides a method of inhibiting a target protein comprising administering to a cell therapeutically effective amount of a compound of formula (I) or a pharmaceutically acceptable salt or a stereoisomer thereof, wherein the compound is effective for inhibiting the target protein.

In certain embodiment, wherein the target protein is MALT-1.

In one embodiment, the present invention provides a method of inhibiting MALT-1 in a subject, comprising administering to the subject in need thereof, a therapeutically effective amount of a compound of formula (I) or a pharmaceutically acceptable salt or a stereoisomer or a tautomer thereof.

In one embodiment, the present invention provides a method of treating a disease or disorder mediated by the inhibition of MALT-1, in a subject comprising administering to the subject in need thereof a therapeutically effective amount of a compound of formula (I) or a pharmaceutically acceptable salt or a stereoisomer or a tautomer thereof.

In one embodiment, the subject is afflicted with a disease or disorder dependent upon MALT-1.

In one embodiment, the subject is afflicted with cancer.

In one embodiment, a disease or disorder dependent on MALT-1, includes cancer.

In one embodiment, the cancer is selected from prostate cancer, brain cancer, breast cancer, colorectal cancer, pancreatic cancer, ovarian cancer, lung cancer, cervical cancer, liver cancer, head/neck/throat cancer, skin cancer, bladder cancer or a hematologic cancer. In one embodiment, cancer may comprise a tumour or a blood born cancer. The tumour may be solid. The tumour is typically malignant and may be metastatic. In one embodiment, the tumour is an adenoma, an adenocarcinoma, a blastoma, a carcinoma, a desmoid tumour, a desmoplastic small round cell tumour, an endocrine tumour, a germ cell tumour, a lymphoma, a leukaemia, a sarcoma, a Wilms tumour, a lung tumour, a colon tumour, a lymph tumour, a breast tumour or a melanoma.

In one embodiment, blastoma comprises hepatoblastoma, glioblastoma, neuroblastoma or retinoblastoma. In one embodiment, carcinoma comprises colorectal carcinoma or hepatocellular carcinoma, pancreatic, prostate, gastric, esophageal, cervical, and head and neck carcinomas, and adenocarcinoma. In one embodiment, types of sarcoma comprise: Ewing sarcoma, osteosarcoma, rhabdomyosarcoma, or any other soft tissue sarcoma. In one embodiment, types of melanoma include Lentigo maligna, Lentigo maligna melanoma, Superficial spreading melanoma, Acral lentiginous melanoma, Mucosal melanoma, Nodular melanoma, Polypoid melanoma, Desmoplastic melanoma, Amelanotic melanoma, Soft-tissue melanoma, Melanoma with small nevus-like cells, Melanoma with features of a Spitz nevus and Uveal melanoma. Types of lymphoma and leukaemia include Precursor T-cell leukemia/lymphoma, acute myeloid leukaemia, chronic myeloid leukaemia, acute lymphocytic leukaemia, Follicular lymphoma, Diffuse large B cell lymphoma, Mantle cell lymphoma, chronic lymphocytic leukemia/lymphoma, MALT lymphoma, Burkitt's lymphoma, Mycosis fungoides, Peripheral T-cell lymphoma, Nodular sclerosis form of Hodgkin lymphoma, Mixed-cellularity subtype of Hodgkin lymphoma. Types of lung tumour include tumours of non-small-cell lung cancer (adenocarcinoma, squamous-cell carcinoma and large-cell carcinoma) and small-cell lung carcinoma.

In one embodiment, the cancer is selected from bladder cancer, colon cancer, hepatocellular cancer, or Small Cell or Non-Small Cell lung cancer. In one embodiment of the invention, the cancer is selected from B-cell malignancies such as B-cell lymphoma, e.g. Diffuse large cell B-cell lymphoma (DLBCL) and Mantle cell lymphoma (MCL), and Leukemias, e.g. chronicle lymphatic leukemia (CLL).

In certain embodiment, the present invention provides a pharmaceutical composition comprising a compound of formula (I) or a pharmaceutically acceptable salt thereof or a stereoisomer thereof as described herein and at least one pharmaceutically acceptable excipient (such as a pharmaceutically acceptable carrier or diluent). Preferably, the pharmaceutical composition comprises a therapeutically effective amount of at least one compound described herein. The compounds described in the present invention may be associated with a pharmaceutically acceptable excipient (such as a carrier or a diluent) or be diluted by a carrier or enclosed within a carrier which can be in the form of a capsule, sachet, paper or other container.

In certain embodiment, the present invention provides a pharmaceutical composition comprising a compound of formula (I), for use in inhibiting a target protein in a cell comprising contacting the cell with an effective amount of the compound, wherein the compound effectuates the inhibition of the target protein.

In certain embodiment, the present invention provides a pharmaceutically acceptable salt or a stereoisomer thereof, for use as a medicament.

In certain embodiment, the present invention provides a pharmaceutical composition comprising a compound of formula (I), for use in the manufacture of a medicament for treating or preventing a disease or disorder mediated by MALT-1.

In certain embodiments, the present invention provides a use of compound of formula (I) or a pharmaceutically acceptable salt or a stereoisomer thereof, in the manufacture of a medicament for treating or preventing a disease or disorder mediated by MALT-1.

In certain embodiments, the present invention provides a pharmaceutical composition comprising a compound of formula (I), for use in treating or preventing of a disease or disorder mediated by MALT-1.

In certain embodiments, the present invention provides a compound of formula (I), for use in treating or preventing of a disease or disorder mediated by MALT-1.

In one embodiment, diseases or disorders dependent on MALT-1, include cancer. In one embodiment, the cancer is selected from bladder cancer, colon cancer, hepatocellular cancer, or Small Cell or Non-Small Cell lung cancer. In one embodiment of the invention, the cancer is selected from B-cell malignancies such as B-cell lymphoma, e.g. Diffuse large cell B-cell lymphoma (DLBCL) and Mantle cell lymphoma (MCL), and Leukemias, e.g. chronicle lymphatic leukemia (CLL).

In certain further embodiment, diseases or disorders dependent on MALT-1, are diffuse large B-cell lymphoma, mantle cell lymphoma, follicular lymphoma, and mucosa-associated lymphoid tissue lymphoma, rheumatoid arthritis, psoriatic arthritis, psoriasis, ulcerative colitis, Crohn's disease, systemic lupus erythematosus, asthma, and chronic obstructive pulmonary disease.

### Pharmaceutical Compositions

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, including but not limited to tableting lubricants and other tableting aids such a magnesium stearate and microcrystalline cellulose. In the case of capsules, tablets and pills, the dosage forms may also comprise buffering agents.

Dosage forms for topical or transdermal administration of a compound of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to an active compound of this application, excipients such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to the compounds of this application, excipients such as lactose, talc, silicic acid, aluminium hydroxide, calcium silicates and polyamide powder or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Administration of the disclosed compounds and pharmaceutical compositions can be accomplished via any mode of administration for therapeutic agents. These modes include systemic or local administration such as oral, nasal, parenteral, intravenous, transdermal, subcutaneous, vaginal, buccal, rectal or topical administration modes.

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising one or more compounds of the present disclosure and a pharmaceutically acceptable carrier, such as, but not limited to, a) a diluent, e.g., purified water, triglyceride oils, such as hydrogenated or partially hydrogenated vegetable oil or mixtures thereof, corn oil, olive oil, sunflower oil, safflower oil, fish oils, such as EPA or DHA or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminium silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, one or more disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles or serum proteins can be used to solubilize the disclosed compounds.

One or more disclosed compounds or compositions can be delivered by parental administration. The parental injectable administration is generally used for subcutaneous, intramuscular or intravenous injections and infusions. Injectables can be prepared in conventional forms, either as liquid solutions or suspensions or solid forms suitable for dissolving in liquid prior to injection.

### Combination Therapy

The compounds of the present invention may be employed either simultaneously with, or before or after, in combination with other anti-cancer agents, e.g. immunomodulating agents, anti-proliferative agents or chemotherapeutic agents or with adjuvants in cancer therapy, e.g. immunosuppressive or anti-inflammatory agents.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

In one embodiment, the present invention provides a composition comprising a MALT-1 blocking agent and one or more anti-cancer agent(s): wherein the MALT-1 blocking agent is represented by a compound of formula (I), (IA), (IB) or (IC), or a compound, salt, stereoisomer or composition as described herein.

In one embodiment, anticancer agents that can be combined with the compounds of the present invention may include, but are not limited to, BTK (Bruton's tyrosine kinase) inhibitors such as ibrutinib, SYK inhibitors (such as Fostamatinib, Entospletinib, Cerdulatinib) PKC inhibitors (such as ruboxistaurin, Tamoxifen), PI3K pathway inhibitors (such as Apitolisib, Gedatolisib, Buparlisib, Copanlisib, Duvelisib, Pictilisib, Taselisib), BCL family inhibitors (such as Oblimersen, navitoclax, venetoclax), JAK inhibitors (such as baricitinib, tofacitinib, and upadacitinib), PIM kinase inhibitors, rituximab or other B cell antigen-binding antibodies, as well as immune cell redirection agents (e.g. blinatumomab or CAR T-cells) and immunomodulatory agents such as daratumumab, anti-PD-1 antibodies, and anti-PD-L1 antibodies.

In certain embodiments, provided herein are methods for combination therapy of a compound of formula (I), (IA), (IB) or (IC), or a compound, salt, stereoisomer or composition as disclosed herein with one or more chemotherapeutic agent, therapeutic antibody, and/or radiation treatment, e.g., to provide a synergistic or additive therapeutic effect for the treatment of diseases, disorders and conditions such as cancer. In certain embodiments, the chemotherapeutic agent is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

For example, further therapeutic agent may comprise an alkylating agent, such as chlorambucil, cyclophosphamide, cisplatin; a mitotic inhibitor such as docetaxel or paclitaxel; an antimetabolite such as 5-fluorouracil, cytarabine, methotrexate, or pemetrexed; an anti-tumor antibiotic such as daunorubicin or doxorubicin; a corticosteroid such as prednisone or methylprednisone; a BCL-2 inhibitor such as venetoclax; or immunotherapeutic compound such as nivolumab, pembrolizumab, pidilizumab, avelumab, BMS 936559, or MPDL3280A, or a combination thereof. In one embodiment, the immunotherapeutic compound comprises chimeric antigen receptor T cells (CAR T-cells).

In certain embodiments, the further therapeutic agent is docetaxel, venetoclax or a hormonal therapy such as fulvestrant. Docetaxel is a type of chemotherapeutic agent known as an antimicrotubule agent. Docetaxel is used for treating a variety of cancers, such as metastatic prostate cancer. Docetaxel treatment is often administered intravenously, and often includes premedication with a corticosteroid such as prednisone. In certain embodiments, the further therapeutic agent is venetoclax which is a BCL-2 inhibitor that can induce apoptosis in cancer cells. Venetoclax is typically administered orally.

In some embodiment, compound of formula (I), (IA), (IB), (IC), or a compound, salt, stereoisomer, tautomer or composition as disclosed herein can be used in combination with one or more chemotherapeutic agents such as, erlotinib, bortezomib, disulfiram, epigallocatechin gallate, salinosporamide A, carfilzomib, 17-AAG (geldanamycin), radicicol, lactate dehydrogenase A (LDH-A), fulvestrant, sunitinib, letrozole, imatinib mesylate, oxaliplatin, 5-FU (5-fluorouracil), Rapamycin, Lapatinib, lonafarnib, sorafenib, gefitinib, anti-metabolites such as methotrexate; taxoids, e.g., paclitaxel, ABRAXANE® (Cremophor-free), albumin-engineered nanoparticle formulations of paclitaxel (American Pharmaceutical Partners, Schaumberg, IL.) and docetaxel/doxetaxel; chlorambucil; gemcitabine; 6-thioguanine; mercaptopurine; platinum analogs such as cisplatin and carboplatin; retinoids such as retinoic acid; and pharmaceutically acceptable salts, acids and derivatives of any of the above.

In some embodiments, compound of formula (I), (IA), (TB) or (IC), or a compound, salt, stereoisomer or composition as disclosed herein can be used in combination with one or more additional pharmaceutical agents such as, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies for treatment of diseases, disorders or conditions, such as cancer.

In some embodiments, compound of formula (I), (IA), (TB) or (IC), or a compound, stereoisomer, salt or composition as disclosed herein can be used in combination therapy with one or more kinase inhibitors for the treatment of cancer. Exemplary kinase inhibitors include imatinib, baricitinib gefitinib, erlotinib, sorafenib, dasatinib, sunitinib, lapatinib, nilotinib, pirfenidone, pazopanib, crizotinib, vemurafenib, vandetanib, ruxolitinib, axitinib, bosutinib, regorafenib, tofacitinib, cabozantinib, ponatinib, trametinib, dabrafenib, afatinib, ibrutinib, ceritinib, idelalisib, nintedanib, palbociclib, lenvatinib, cobimetinib, abemaciclib, acalabrutinib, alectinib, binimetinib, brigatinib, encorafenib, erdafitinib, everolimus, fostamatinib, gilterinib, larotrectinib, lorlatinib, netarsudil, osimertinib, pemigatinib, pexidartinib, ribociclib, temsirolimus, XL-102, XL-092, XL-147, XL-765, XL-499, and XL-880.

In some embodiments, a compound of formula (I), (IA), (IB) or (IC), or a compound, salt, stereoisomer or composition as disclosed herein can be used in combination with a HSP90 inhibitor (e.g., XL888), liver X receptor (LXR) modulators, retinoid-related orphan receptor gamma (RORγ) modulators, checkpoint inhibitors such as a CK1 inhibitor or a CK1α inhibitor, a Wnt pathway inhibitor (e.g., SST-215) or a mineralocorticoid receptor inhibitor, (e.g., esaxerenone) or Poly ADP ribose polymerase (PARP) inhibitors, such as, olaparib, rucaparib, niraparib, talazoparib for the treatment of cancer.

In some embodiments, for the treatment of cancer, a compound of formula (I), (IA), (IB) or (IC), or a compound, salt, stereoisomer or composition as disclosed herein can be used in combination with one or more immune check point inhibitors, for example, inhibitors of PD-1 or inhibitors of PD-L1, e.g., an anti-PD-1 monoclonal antibody or an anti-PD-L1 monoclonal antibody, for example, nivolumab (Opdivo®), pembrolizumab (Keytruda®, MK-3475), atezolizumab, avelumab, cemiplimab, spartalizumab, camrelizumab, cetrelimab, toripalimab, sintilimab, AB122, JTX-4014, BGB-108, BCD-100, BAT1306, LZMO09, AK105, HLX10, and TSR-042, AMP-224, AMP-514, PDR001, durvalumab, pidilizumab (Imfinzi®, CT-011), CK-301, BMS 936559, and MPDL3280A. In some embodiments, the anti-PD-1 monoclonal antibody is nivolumab, pembrolizumab, pidilizumab, PDR001, MGA012, PDR001, AB122, or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab.

In some embodiments, for the treatment of cancer, compound of formula (I), (IA), (TB) or (IC), or a compound, salt, stereoisomer or composition as disclosed herein can be used in combination with one or more inhibitors of PD-L1. Antibodies that bind to human PD-L1 include atezolizumab, avelumab, durvalumab, tislelizumab, BMS-935559, MEDI4736, FAZ053, KN035, CS1001, CBT-502, A167, STI-A101, CK-301, BGB-A333, MSB-2311, HLX20, KN035, and LY3300054. In some embodiments, the anti-PD-L1 monoclonal antibody is BMS-935559, MEDI4736, MPDL3280A, or MSB0010718C. In some embodiments, the anti-PD-L1 monoclonal antibody is atezolizumab, avelumab, durvalumab.

In some embodiments, for treatment of cancer, a compound of formula (I), (IA), (TB) or (IC), or a compound, salt, stereoisomer or composition as disclosed herein can be used in combination with one or more CTLA-4 inhibitors, e.g., an anti-CTLA-4 antibody, for example, ipilimumab (Yervoy®), tremelimumab and AGEN1884; and phosphatidylserine inhibitors, for example, bavituximab (PGN401); antibodies to cytokines (IL-10, TGF-b, and the like.); other anti-cancer agents such as cemiplimab. In some embodiments, the inhibitor of an immune checkpoint molecule is an inhibitor of PD-L1 and CTLA-4, e.g., an anti-PD-LI/CTLA-4 bispecific antibody or an anti-PD-1/CTLA-4 bispecific antibody. Bispecific antibodies that bind to PD-L1 and CTLA-4 include AK104.

In certain embodiments, the present invention provides a composition comprising a compound of formula (I), (IA), (IB) or (IC), or a compound, salt, stereoisomer or composition as disclosed herein in combination with one or more other therapeutic agents and a pharmaceutically acceptable excipient or carrier. The compound of formula (I), (IA), (IB) or (IC), or a compound, salt or composition as disclosed herein may be administered in combination with one or more other therapeutic agents (preferably one or two, more preferably one): (1) to complement and/or enhance prevention and/or therapeutic efficacy of the preventive and/or therapeutic drug effect of the compound of the present invention, (2) to modulate pharmacodynamics, improve absorption improvement, or reduce dosage reduction of the preventive and/or therapeutic compound of the present invention, and/or (3) to reduce or ameliorate the side effects of the preventive and/or therapeutic compound of the present invention. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds).

For example, the different therapeutic compounds can be administered either in the same formulation or in a separate formulation, concomitantly, sequentially or separately. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds. The respective compounds may be administered by the same or different route and the same or different method. In certain embodiments, the other therapeutic compounds can be administered within one hour, 12 hours, 24 hours, 36 hours, 48 hours, 72 hours, or a week prior to or after administration of a compound of formula (I), (IA), (IB) or (IC), or a compound, salt, stereoisomer or composition as disclosed herein. In certain embodiments, the other therapeutic compounds can be administered within 0.5 hours to 24 hours prior to or after administration of a compound of formula (I), (IA), (IB) or (IC), or a compound, salt, stereoisomer or composition as disclosed herein. In certain embodiments, the other therapeutic compounds can be administered within 0.5 hours to 72 hours prior to or after administration of a compound of formula (I), (IA), (IB) or (IC), or a compound, salt, stereoisomer or composition as disclosed herein. In certain embodiments, the other therapeutic compounds can be administered within 2 hours prior to or after administration of a compound of formula (I), (IA), (IB) or (IC), or a compound, salt or composition as disclosed herein.

A concomitant medicine comprising the compound of the present invention and other drug may be administered as a combination preparation in which both components are contained in a single formulation or administered as separate formulations. The administration by separate formulations includes simultaneous administration and or administration of the formulations separated by some time intervals. In the case of the administration with some time intervals, the compound of the present invention can be administered first, followed by another drug or another drug can be administered first, followed by the compound of the present invention, so long as the two compounds are simultaneously active in the patient at least some of the time during the conjoint therapy. The administration method of the respective drugs may be administered by the same or different route and the same or different method.

The dosage of the other drug can be properly selected, based on a dosage that has been clinically used, or may be a reduced dosage that is effective when administered in combination with a compound of the present invention. The compounding ratio of the compound of the present invention and the other drug can be properly selected according to age and weight of a subject to be administered, administration method, administration time, disorder to be treated, symptom and combination thereof. For example, the other drug may be used in an amount of about 0.01 to about 100 parts by mass, based on 1 part by mass of the compound of the present invention. The other drug may be a combination of two or more kind of arbitrary drugs in a proper proportion. The other drug that complements and/or enhances the preventive and/or therapeutic efficacy of the compound of the present invention includes not only those that have already been discovered, but those that will be discovered in future, based on the above mechanism.

In certain embodiments, the present compound of formula (I), (IA), (IB) or (IC), or a compound, salt, stereoisomer or composition as disclosed herein thereof may be conjointly administered with non-chemical methods of cancer treatment. In certain embodiments, the compound of formula (I), (IA), (TB) or (IC), or a compound, salt, stereoisomer or composition as disclosed herein can be conjointly administered with non-chemical methods of cancer treatment. In certain embodiments, the compound of formula (I), (IA), (IB) or (IC), or a compound, salt, stereoisomer or composition as disclosed herein can be conjointly administered with radiation therapy. In certain embodiments, the compound of formula (I), (IA), (IB) or (IC), or a compound, salt, stereoisomer or composition as disclosed herein can be conjointly administered with surgery, with thermoablation, with focused ultrasound therapy, with cryotherapy, or with any combination of these.

### Definitions

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The singular forms “a”, “an” and “the” encompass plural references unless the context clearly indicates otherwise.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to an event or circumstance in which the said alkyl may be substituted as well as the event or circumstance in which the alkyl is not substituted. The term “optionally substituted alkyl” can also be referred to ‘unsubstituted or substituted alkyl’ group, wherein the substituents unless specifically stated therein, are

The term “substituted” refers to moieties having substituents replacing hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Unless specifically stated, the substituents can include any substituents described herein, for example, a halogen, a hydroxyl, a carbonyl (such as a carboxyl, an alkoxycarbonyl, a formyl or an acyl), a thiocarbonyl (such as a thioester, a thioacetate or a thioformate), an alkoxyl, an oxo, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heteroaryl, a heterocycloalkyl, an aralkyl or an aromatic or heteroaromatic moiety. It will be understood by those skilled in the art that substituents can themselves be substituted, if appropriate.

As used herein, the term “alkyl” refers to saturated aliphatic groups, including but not limited to C₁-C₁₀ straight-chain alkyl groups or C₃-C₁₀ branched-chain alkyl groups. Preferably, the “alkyl” group refers to C₁-C₆ straight-chain alkyl groups or C₃-C₆ branched-chain alkyl groups. Most preferably, the “alkyl” group refers to C₁-C₄ straight-chain alkyl groups or C₃-C₈ branched-chain alkyl groups. Examples of “alkyl” include, but are not limited to, methyl, ethyl, 1-propyl, 2-propyl, isopropyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl and 4-octyl. The “alkyl” group may be optionally substituted.

As used herein, the term “halo” or “halogen” alone or in combination with other term(s) means fluorine, chlorine, bromine or iodine.

As used herein, the term “haloalkyl” refers to alkyl substituted with one or more halogen atoms, wherein the halo and alkyl groups are as defined above. Examples of “haloalkyl” include but are not limited to fluoromethyl, difluoromethyl, chloromethyl, trifluoromethyl and 2,2,2-trifluoroethyl.

As used herein, the term “hydroxyalkyl” refers to an alkyl group, as defined above, wherein one or more of the alkyl group's hydrogen atoms have been replaced with hydroxyl group. Examples of hydroxyalkyl moieties include but are not limited to —CH₂OH, —CH₂CH₂OH, —CH₂CH₂CH₂OH, —CH₂CH(OH)CH₂OH, —CH₂CH(OH) CH₃, —CH(CH₃)CH₂OH.

As used herein, the term “heterocycloalkyl” refers to a non-aromatic, saturated or partially saturated, bridged bicyclic, spirocyclic, monocyclic or polycyclic ring system of 3 to 15 member, unless the ring size is specifically mentioned, having at least one heteroatom or hetero group selected from O, N, S, S(O), S(O)₂, NH or C(O) with the remaining ring atoms being independently selected from the group consisting of carbon, oxygen, nitrogen, and sulfur. The term “heterocycloalkyl” also refers to the bridged bicyclic ring system having at least one heteroatom or hetero group selected from O, N, S, S(O), S(O)₂, NH or C(O). Examples of “heterocycloalkyl” include, but not limited to, azetidinyl, oxetanyl, imidazolidinyl, pyrrolidinyl, oxazolidinyl, thiazolidinyl, pyrazolidinyl, tetrahydrofuranyl, piperidinyl, dihydropyridinyl, piperazinyl, tetrahydropyranyl, morpholinyl, thiomorpholinyl, 1,4-dioxanyl, dioxidothiomorpholinyl, oxopiperazinyl, oxopiperidinyl, tetrahydrofuryl, tetrahydropyranyl, tetrahydrothiophenyl, dihydropyranyl, indolinyl, indolinylmethyl, isoindolinyl, oxoisoindolinyl, dioxoisoindolinyl, aza-bicyclooctanyl, diazabicyclooctanyl, azocinyl, chromanyl, isochromanyl, xanthenyl and 2-oxa-6-azaspiro[3.3]heptanyl. Attachment of a heterocycloalkyl substituent can occur via either a carbon atom or a heteroatom. A heterocycloalkyl group can be optionally substituted with one or more suitable groups by one or more aforesaid groups. Preferably “heterocycloalkyl” refers to 5- to 6-membered ring (unless the ring size is specifically mentioned) selected from the group consisting of azetidinyl, oxetanyl, imidazolidinyl, pyrrolidinyl, oxazolidinyl, thiazolidinyl, pyrazolidinyl, tetrahydrofuranyl, piperidinyl, piperazinyl, tetrahydropyranyl, morpholinyl and thiomorpholinyl. All heterocycloalkyl are optionally substituted by one or more aforesaid groups.

As used herein, the term “heteroaryl” refers to a completely unsaturated ring system containing a total of 5 to 14 ring atoms, unless the ring size is specifically mentioned. At least one of the ring atoms is a heteroatom (i.e., O, N or S), with the remaining ring atoms/groups being independently selected from C, N, O or S. A heteroaryl may be a single-ring (monocyclic) or multiple rings (bicyclic, tricyclic or polycyclic) fused together or linked covalently. Preferably, “heteroaryl” is a 5- to 6-membered ring, unless the ring size is specifically mentioned. The rings may contain from 1 to 4 additional heteroatoms selected from N, O and S, wherein the N atom is optionally quarternized. Any suitable ring position of the heteroaryl moiety may be covalently linked to the defined chemical structure. Examples of “heteroaryl” include but not limited to furanyl, thienyl, pyrrolyl, pyrazolyl, imidazolyl, oxazolyl, cinnolinyl, isoxazolyl, thiazolyl, isothiazolyl, 1H-tetrazolyl, oxadiazolyl, triazolyl, pyridyl (pyridinyl), 3-fluoropyridyl, pyrimidinyl, pyrazinyl, pyridazinyl, benzoxazolyl, benzisoxazolyl, benzothiazolyl, benzofuranyl, benzothienyl, benzotriazinyl, phthalazinyl, thianthrene, dibenzofuranyl, dibenzothienyl, benzimidazolyl, indolyl, isoindolyl, indazolyl, quinolinyl, isoquinolinyl, quinazolinyl, quinoxalinyl, purinyl, pteridinyl, 9H-carbazolyl, α-carbolinyl, indolizinyl, benzoisothiazolyl, benzoxazolyl, pyrrolopyridyl, purinyl, benzothiadiazolyl, benzooxadiazolyl, benzotriazolyl, benzotriadiazolyl, carbazolyl, dibenzothienyl, acridinyl and the like. Heteroaryl group may be optionally further substituted.

As used herein, the term “heterocyclyl” or “heterocyclic” alone or in combination with other term(s) includes both “heterocycloalkyl” and “heteroaryl” groups which are as defined above. Accordingly, heterocyclic groups may be optionally further substituted.

As used herein, the term “amino” refers to an —NH₂ group.

As used herein, the term “hydroxy” or “hydroxyl” alone or in combination with other term(s) means —OH.

As used herein, the term “oxo” refers to ═O group.

As used herein, the term “alkoxy” refers to the group —O-alkyl, where alkyl groups are as defined above. Exemplary C₁-C₁₀ alkoxy group include but are not limited to methoxy, ethoxy, n-propoxy, n-butoxy or t-butoxy. An alkoxy group can be optionally substituted with one or more suitable groups.

The term “heteroatom” as used herein designates a sulfur, nitrogen or oxygen atom.

As used herein, the term ‘compound(s)’ comprises the compound(s) disclosed in the present invention.

As used herein, the term “comprise” or “comprising” is generally used in the sense of include, that is to say permitting the presence of one or more features or components.

As used herein, the term “or” means “and/or” unless stated otherwise.

As used herein, the term “including” as well as other forms, such as “include”, “includes” and “included” is not limiting.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

As used herein, the term “pharmaceutical composition” refers to a composition(s) containing a therapeutically effective amount of at least one compound of formula (I) or a pharmaceutically acceptable salt thereof and a pharmaceutically acceptable carrier.

The pharmaceutical composition(s) usually contain(s) about 1% to 99%, for example, about 5% to 75% or from about 10% to about 30% by weight of the compound of formula (I) or (II) or pharmaceutically acceptable salts thereof. The amount of the compound of formula (I) or pharmaceutically acceptable salts thereof in the pharmaceutical composition(s) can range from about 1 mg to about 1000 mg or from about 2.5 mg to about 500 mg or from about 5 mg to about 250 mg or in any range falling within the broader range of 1 mg to 1000 mg or higher or lower than the aforementioned range.

As used herein, “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant or emulsifier that has been approved by the United States Food and Drug Administration as being acceptable for use in humans or domestic animals.

The term “administer,” “administering,” or “administration” as used in this disclosure refers to either directly administering one or more disclosed compounds or a pharmaceutically acceptable salt of one or more disclosed compounds or a composition comprising one or more disclosed compounds to a subject or analog of the compound or a pharmaceutically acceptable salt of the compound or composition to the subject, which can form an equivalent amount of active compound within the subject's body.

The term “carrier” as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ or portion of the body, to another organ or portion of the body of a subject.

As used herein, the term “treat”, “treating” and “treatment” refer to a method of alleviating or abrogating a disease and/or its attendant symptoms.

As used herein, the term “prevent”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

As used herein, the term “subject” that may be interchangeable with ‘patient’, refers to an animal, preferably a mammal, and most preferably a human.

As used herein, the term, “therapeutically effective amount” refers to an amount of a compound of formula (I) or a pharmaceutically acceptable salt or a stereoisomer thereof; or a composition comprising the compound of formula (I) or a pharmaceutically acceptable salt or a stereoisomer thereof, effective in producing the desired therapeutic response in a particular patient suffering from a diseases or disorder, in particular their use in diseases or disorder associated with cancer. Particularly, the term “therapeutically effective amount” includes the amount of the compound of formula (I) or a pharmaceutically acceptable salt or a stereoisomer thereof, when administered, that induces a positive modification in the disease or disorder to be treated or is sufficient to prevent development of or alleviate to some extent, one or more of the symptoms of the disease or disorder being treated in a subject. In respect of the therapeutic amount of the compound, the amount of the compound used for the treatment of a subject is low enough to avoid undue or severe side effects, within the scope of sound medical judgment can also be considered. The therapeutically effective amount of the compound or composition will be varied with the particular condition being treated, the severity of the condition being treated or prevented, the duration of the treatment, the nature of concurrent therapy, the age and physical condition of the end user, the specific compound or composition employed the particular pharmaceutically acceptable carrier utilized.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

The term “pharmaceutically acceptable salt” refers to a product obtained by reaction of the compound of the present invention with a suitable acid or a base. In some cases, a medicament can be present in the form of a pharmaceutically acceptable salt. In some instances, a pharmaceutically acceptable salt can be a salt described in Berge et al, J. Pharm. Sci, 1977. In some instances, a pharmaceutically acceptable salts can include those salts derived from a mineral, organic acid or inorganic base. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids.

The pharmaceutically acceptable salts of the present invention can be prepared from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20^{th} ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

The present invention also provides methods for formulating the disclosed compounds as for pharmaceutical administration.

In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

The term “cancer” is used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated. As used herein, the term neoplasia is used to describe all cancerous disease states and embraces or encompasses the pathological process associated with malignant hematogenous, ascitic and solid tumors. Exemplary cancers which may be treated by the present compounds either alone or in combination with at least one additional anti-cancer agent include, but not limited to: Cardiac: sarcoma (angiosarcoma, fibrosarcoma, rhabdomyosarcoma, liposarcoma), myxoma, rhabdomyoma, fibroma, lipoma and teratoma; Head and neck: squamous cell carcinomas of the head and neck, laryngeal and hypopharyngeal cancer, nasal cavity and paranasal sinus cancer, nasopharyngeal cancer, salivary gland cancer, oral and oropharyngeal cancer; Lung:

bronchogenic carcinoma (squamous cell, undifferentiated small cell, undifferentiated large cell, adenocarcinoma, non-small cell lung cancer), alveolar (bronchiolar) carcinoma, bronchial adenoma, sarcoma, lymphoma, chondromatous hamartoma, mesothelioma; Colon: colorectal cancer, adenocarcinoma, gastrointestinal stromal tumors, lymphoma, carcinoids, Turcot Syndrome; Gastrointestinal: gastric cancer, gastroesophageal junction adenocarcinoma, esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Breast: metastatic breast cancer, ductal carcinoma in situ, invasive ductal carcinoma, tubular carcinoma, medullary carcinoma, mucinous carcinoma, lobular carcinoma in situ, triple negative breast cancer; Genitourinary tract: kidney (adenocarcinoma, Wilm's tumor [nephroblastoma], lymphoma, leukemia, renal cell carcinoma), bladder and urethra (squamous cell carcinoma, transitional cell carcinoma, adenocarcinoma, urothelial carcinoma), prostate (adenocarcinoma, sarcoma, castrate resistant prostate cancer), testis (seminoma, teratoma, embryonal carcinoma, teratocarcinoma, choriocarcinoma, sarcoma, interstitial cell carcinoma, fibroma, fibroadenoma, adenomatoid tumors, lipoma), clear cell carcinoma, clear cell renal cell carcinoma, non-clear cell renal cell carcinoma, papillary carcinoma; Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma, and giant cell tumors; Thyroid: medullary thyroid cancer, differentiated thyroid cancer, papillary thyroid cancer, follicular thyroid cancer, Hurthle cell cancer, and anaplastic thyroid cancer; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial cancer), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

The term “stereoisomers” refers to any enantiomers, diastereoisomers or geometrical isomers of the compound of formula (I), wherever they are chiral or when they bear one or more double bonds. When the compounds of the formula (I) and related formulae are chiral, they can exist in racemic or in optically active form. It should be understood that the invention encompasses all stereochemical isomeric forms, including diastereomeric, enantiomeric and epimeric forms, as well as d-Isomers and l-Isomers and mixtures thereof. Individual stereoisomers of compounds can be prepared synthetically from commercially available starting materials which contain chiral centres or by preparation of mixtures of enantiomeric products followed by separation such as conversion to a mixture of diastereomers followed by separation or recrystallization, chromatographic techniques, direct separation of enantiomers on chiral chromatographic columns or any other appropriate method known in the art. Starting compounds of particular stereochemistry are either commercially available or can be made and resolved by techniques known in the art. Additionally, the compounds of the present invention may exist as geometric Isomers. The present invention includes all cis, trans, syn, anti, entgegen (E) and zusammen (Z) Isomers as well as the appropriate mixtures thereof.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers. The disclosure includes enantiomers of the compounds described herein. Each compound herein disclosed includes all the enantiomers that conform to the general structure of the compound. The compounds may be in a racemic or enantiomerically pure form or any other form in terms of stereochemistry. In some embodiments the compounds are the (R, S)-enantiomer.

The term “diastereomers” refers to the set of stereoisomers which cannot be made superimposable by rotation around single bonds. For example, cis- and trans- double bonds, endo- and exo- substitution on bicyclic ring systems, and compounds containing multiple stereogenic centres with different relative configurations are considered to be diastereomers. The term “diastereomer” refers to any member of this set of compounds. In some examples presented, the synthetic route may produce a single diastereomer or a mixture of diastereomers. The disclosure includes diastereomers of the compounds described herein.

The compounds of the present invention may be used as single drug or as a pharmaceutical composition in which the compound is mixed with various pharmacologically acceptable excipients.

The compounds of the invention are typically administered in the form of a pharmaceutical composition. Such compositions can be prepared using procedures well known in the pharmaceutical art and comprise at least one compound of the invention. The pharmaceutical composition of the present patent application comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents and solvents.

The pharmaceutical composition can be administered by oral, parenteral or inhalation routes. Examples of the parenteral administration include administration by injection, percutaneous, transmucosal, transnasal and transpulmonary administrations.

Examples of suitable carriers include, but are not limited to, water, salt solutions, alcohols, polyethylene glycols, peanut oil, olive oil, gelatin, lactose, terra alba, sucrose, dextrin, magnesium carbonate, sugar, amylose, magnesium stearate, talc, gelatin, agar, pectin, acacia, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing.

The pharmaceutical compositions may be in conventional forms, for example, tablets, capsules, solutions, suspensions, injectables or products for topical application. Further, the pharmaceutical composition of the present invention may be formulated so as to provide desired release profile.

Administration of the compounds of the invention, in pure form or in an appropriate pharmaceutical composition, can be carried out using any of the accepted routes of administration of pharmaceutical compositions. The route of administration may be any route which effectively transports the active compound of the patent application to the appropriate or desired site of action. Suitable routes of administration include, but are not limited to oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions, and sterile injectable liquids, such as suspensions or solutions.

Topical dosage forms of the compounds include ointments, pastes, creams, lotions, powders, solutions, eye or ear drops, impregnated dressings, and may contain appropriate conventional additives such as preservatives, solvents to assist drug penetration.

The pharmaceutical compositions of the present patent application may be prepared by conventional techniques known in literature.

Suitable doses of the compounds for use in treating the diseases or disorders described herein can be determined by those skilled in the relevant art. Therapeutic doses are generally identified through a dose ranging study in humans based on preliminary evidence derived from the animal studies. Doses must be sufficient to result in a desired therapeutic benefit without causing unwanted side effects. Mode of administration, dosage forms, and suitable pharmaceutical excipients can also be well used and adjusted by those skilled in the art. All changes and modifications are envisioned within the scope of the present patent application.

According to one embodiment, the compounds of the present invention can also contain unnatural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention, and their uses. Exemplary isotopes that can be incorporated into compounds of the invention include isotopes of hydrogen, carbon, nitrogen, oxygen, phosphorous, sulfur, fluorine, chlorine and iodine, such as ²H (“D”), ³H, ¹¹C, ¹³C, ¹⁴C, ¹³N, ¹⁵N, ¹⁵O, ¹⁷O, ¹⁸O, ³²P, ³³P ³⁵S, ¹⁸F, ³⁶Cl, ¹²³I and ¹²⁵I. Isotopically labeled compounds of the present inventions can generally be prepared by following procedures analogous to those disclosed in the schemes and/or in the examples herein below, by substituting an isotopically labeled reagent for a non-isotopically labeled reagent.

### Experimental

### The Following Abbreviations Refer Respectively to the Definitions Below:

DMSO—Dimethylsulfoxide; DIPEA—N,N-Diisopropylethylamine; NaHCO₃—Sodium bicarbonate; EtOH—Ethanol; MeOH—Methanol; THF—Tetrahydrofuran; SOCl₂—Thionyl chloride; LAH—Lithium aluminium hydride; NaH—Sodium hydride; MnO₂—Manganese dioxide; IPA—Isopropyl alcohol; NaBH₄—Sodium borohydride; KO^{t}Bu—Potassium tert-butoxide; TFA—Trifluoro acetic acid; TBSOTf—tert-Butyldimethylsilyl trifluoromethanesulfonate; TBAI—Tetra butyl ammonium iodide; K₂CO₃—Potassium carbonate; EDCI—1-Ethyl-3-(3-dimethylaminopropyl)carbodiimide; DMAP—4-Dimethylaminopyridine; PBr₃—Phosphorous tribromide; NH₄OAc—Ammonium acetate; TMSOTf—Trimethylsilyl trifluoromethanesulfonate; Pd(dppf)Cl₂—[1,1′-Bis(diphenylphosphino)ferrocene]palladium(II) dichloride; Cs₂CO₃—Caesium carbonate; OsO₄—Osmium tetroxide; NaIO₄—Sodium per iodate; CBr₄—Carbon tetra bromide; PPh₃—Triphenyl phosphine; Pd₂(dba)₃—Tris(dibenzylideneacetone)dipalladium(O); dppf—1,1′-Bis(diphenylphosphino)ferrocene; Zn(CN)₂—Zinc cyanide; CuI—Copper Iodide; DMEDA—N,N-Dimethylethylenediamine; HATU—1-[Bis(dimethylamino)methylene]-1H-1,2,3-triazolo[4,5-b]pyridinium 3-oxid hexafluorophosphate; NH₄Cl—Ammonium chloride; Al₂O₃—Aluminium oxide; Zn—Zinc; NBS—N-Bromo succinimide; AIBN—azobisisobutyronitrile; CCl₄—Carbon tetra chloride; DME—Dimethoxy ethane; P(Cy)₃—Tricyclohexylphosphine; Pd(OAc)₂—Palladium(II) acetate ; K₃PO₄—Tripotassium phosphate; CDI—1,1′-Carbonyldiimidazole; KI—Potassium iodide; NaOEt—Sodium ethoxide; (Boc)₂O—Di-tert-butyl dicarbonate; MeI—Methyl iodide; Dioxane.HCl;—Hydrochloric acid in dioxane; Na₂SO₄—Sodium sulphate; Na₂CO₃—Sodium carbonate; Na₂S₂O₃—sodium thiosulphate; H₂O—water; br—Broad; Å—Angstrom ; ° C.—Degree Celsius ; conc—Concentrated; CHCl₃—Chloroform; CDCl₃//chloroform-d—Deuterated Chloroform; DMSO-d₆—Deuterated dimethylsulfoxide; CH₂Cl₂—DCM—Dichloromethane; DMF—N,N-Dimethylformamide;; Et₂O—Diethyl ether; g—Gram; h—Hours; ¹H—Proton; HCl—Hydrochloric acid; Hz—Hertz; J—Coupling Constant; LC-MS—Liquid Chromatography—Mass Spectroscopy; HPLC—High-performance liquid chromatography; chiral HPLC—chiral high-performance liquid chromatography; M—Molar; MHz—Mega Hertz (frequency); MS—Mass Spectroscopy; mmol—Milli Mole; mL—Milli Litre; min—Minutes; mol—Moles; M⁺—Molecular ion; m/z—mass to charge ratio; N—Normality; NMR—Nuclear Magnetic Resonance; Et₃N/TEA—Triethyl amine; ppm—Parts per million; rt/RT—Room temperature; s—Singlet; d—Doublet, t—Triplet; q—Quartet; m—Multiplet; dd—doublet of doublets; td—triplet of doublets; qd—quartet of doublets; ddd—doublet of doublet of doublets; dt—doublet of triplets; ddt—doublet of doublet of triplets; p—pentet; TLC—Thin Layer Chromatography; THF—Tetrahydrofuran; %—Percentage; μ—Micron; μL—Microliter and δ—Delta; anh.—anhydrous. ±—racemic mixture; SFC—Super critical fluid chromatography.

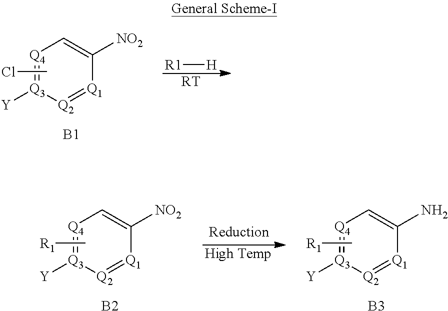

The general scheme for the synthesis of a compound of formula (B3) is depicted in above scheme. The compound of formula (B1) can undergo substitution reaction in the presence of solvent at room temperature to result a compound of formula (B2) which upon reduction at high temperature affords a compound of formula (B3).

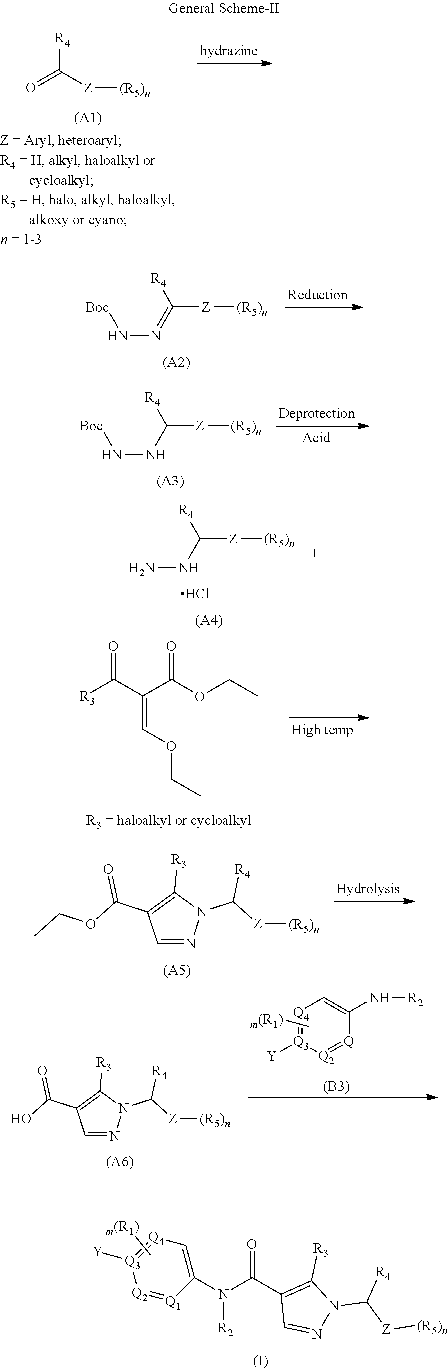

The general scheme for the synthesis of compound of formula (I) is depicted in above scheme. The imine compound of formula (A2) can be obtained from compound of formula (A1) by reacting with hydrazine in an appropriate solvent. The compound of formula (A2) upon reduction with suitable reducing agents at room temperature can afford compound of formula (A3) which can be deprotected in the presence of acid to result in compound of formula (A4). The hydrazine derivative of formula (A4) can be reacted with an alkoxyethylene β-ketoester intermediate to yield a pyrazole-containing intermediate of formula (A5) which can be hydrolysed to provide an acid intermediate of formula (A6) which upon reaction with respective amine of formula B3 provides the compound of formula (I).

Intermediate-1: 5-Amino-2-(2H-1,2,3-triazol-2-yl) nicotinonitrile

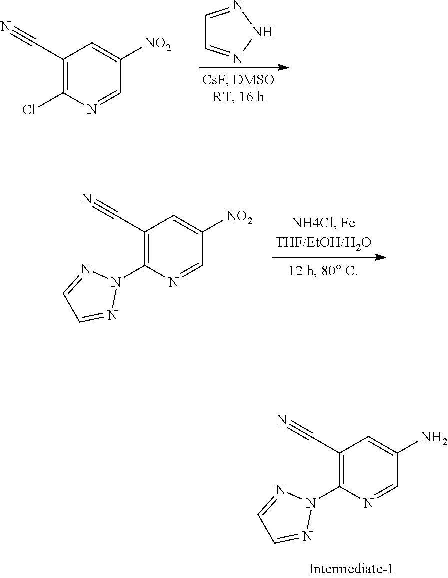 [SMILES: N#Cc1cc([N+](=O)[O-])cnc1Cl ; c1cn[nH]n1 ; N#Cc1cc([N+](=O)[O-])cnc1-n1nccn1 ; N#Cc1cc(N)cnc1-n1nccn1]

Step-a: Synthesis of 5-nitro-2-(2H-1,2,3-triazol-2-yl) nicotinonitrile

To a stirred solution of 2-chloro-5-nitronicotinonitrile (5.0 g, 27.239 mmol), 1H-1,2,3-triazole (3.759 g, 54.480 mmol) in 50 mL of DMSO was added cesium fluoride (8.276 g, 54.480 mmol). The reaction mixture was stirred for 16 hours under nitrogen atmosphere at room temperature. Then, to the reaction mixture, water was added. The precipitated solid was filtered and dried under reduced pressure to obtain 4.1 g (69.63%) of 5-nitro-2-(2H-1,2,3-triazol-2-yl) nicotinonitrile. ¹H NMR (300 MHz, DMSO-d6) δ 9.62 (d, J=2.6 Hz, 1H), 9.46 (d, J=2.7 Hz, 1H), 8.47 (s, 2H).; LCMS: 217.1 [M+H]⁺.

Step-b: Synthesis of 5-amino-2-(2H-1,2,3-triazol-2-yl) nicotinonitrile

To a stirred solution of 5-nitro-2-(2H-1,2,3-triazol-2-yl) nicotinonitrile (4.0 g, 18.504 mmol) in 30 mL of ethanol were added THF (30.0 mL), ammonium chloride (4.949 g, 95.52 mmol) in water (30 mL) and Fe powder (5.181 g, 92.520 mmol). The reaction mixture was stirred for 12 hours at 80° C. The reaction mixture was filtered through Celite® bed and filtrate was concentrated. The obtained residue was diluted with ethyl acetate. The ethyl acetate layer was washed with water and brine solution then dried over anhydrous sodium sulphate and concentrated under reduced pressure to obtain 2.5 g (72.57%) of 5-amino-2-(2H-1,2,3-triazol-2-yl) nicotinonitrile. ¹H NMR (400 MHz, DMSO-d6) δ 8.18—8.09 (m, 3H), 7.47 (d, J=2.8 Hz, 1H), 6.28 (s, 2H). LCMS: 187.1 [M+H]⁺.

Intermediate-2: 5-Chloro-6-(2H-1,2,3-triazol-2-yl) pyridin-3-amine

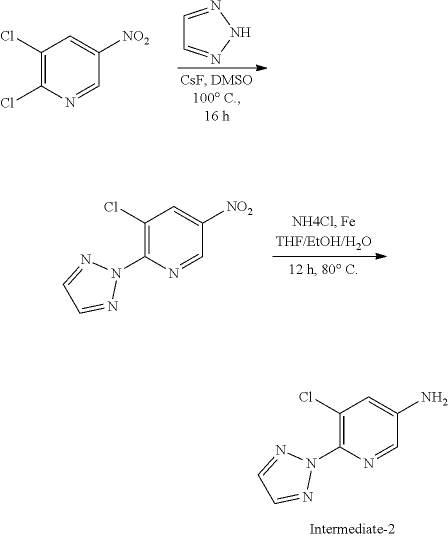 [SMILES: O=[N+]([O-])c1cnc(Cl)c(Cl)c1 ; c1cn[nH]n1 ; O=[N+]([O-])c1cnc(-n2nccn2)c(Cl)c1 ; Nc1cnc(-n2nccn2)c(Cl)c1]

Step-a: Synthesis of 5-chloro-6-(2H-1,2,3-triazol-2-yl) pyridin-3-amine

To a stirred solution of 2,3-dichloro-5-nitropyridine (5.0 g, 25.908 mmol), 1H-1,2,3-triazole (2.684 g, 38.864 mmol) in 50 mL of DMSO, was added cesium fluoride (3.936 g, 25.910 mmol). The reaction mixture was stirred for 16 hours under nitrogen atmosphere at 100° C. To the reaction mixture, water was added and the precipitated solid was filtered and dried under reduced pressure to obtain 3.6 g (61.60%) of 3-chloro-5-nitro-2-(2H-1,2,3-triazol-2-yl) pyridine. ¹H NMR (300 MHz, DMSO-d6) δ 9.39 (d, J=2.4 Hz, 1H), 9.15 (d, J=2.4 Hz, 1H), 8.34 (d, J=2.9 Hz, 2H).; LCMS: 226.0 [M+H]⁺.

Step-b: Synthesis of 5-chloro-6-(2H-1,2,3-triazol-2-yl) pyridin-3-amine

To a stirred solution of 3-chloro-5-nitro-2-(2H-1,2,3-triazol-2-yl)pyridine (3.6 g, 15.958 mmol) in 10 mL of ethanol, were added THF (10.0 mL), ammonium chloride (4.268 g, 79.790 mmol) in water (10 mL) and Fe powder (4.456 g, 79.790 mmol). The reaction mixture was stirred for 12 hours at 80° C. The reaction mixture was filtered through Celite bed and the filtrate was concentrated. The obtained residue was diluted with ethyl acetate. The ethyl acetate layer was washed with water and brine solution then dried over anhydrous sodium sulphate and concentrated under reduced pressure to obtain 2.6 g (83.29%) of 5-chloro-6-(2H-1,2,3-triazol-2-yl) pyridin-3-amine. ¹H NMR (300 MHz, DMSO-d6) δ 8.05 (s, 2H), 7.82 (d, J=2.4 Hz, 1H), 7.20 (d, J=2.4 Hz, 1H), 6.20 (s, 2H).; LCMS: 196.0 [M+H]⁺.

### Example-1

### 1-(2-chloro-3-fluorobenzyl)-N-(5-cyano-6-(2H-1,2,3-triazol-2-yl) pyridin-3-yl)-5-(trifluoromethyl)-1H-pyrazole-4-carboxamide

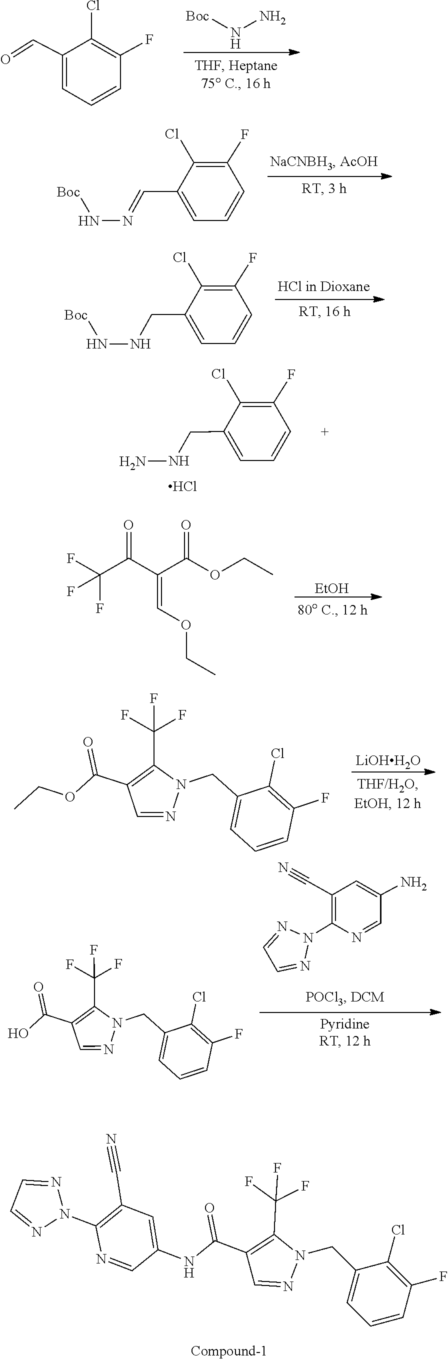

Step-a: Synthesis of tert-butyl (E)-2-(2-chloro-3-fluorobenzylidene) hydrazine-1-carboxylate

To a stirred solution of 2-chloro-3-fluorobenzaldehyde (3.0 g, 18.92 mmol) in 8.0 mL of THF was added heptane (16.0 mL) followed by tert-butyl hydrazine carboxylate (2.5 g, 18.92 mmol). The reaction mixture was stirred for 16 hours at 75° C. The reaction mixture was concentrated under reduced pressure to obtain 5.1 g (98.85%) of tert-butyl (E)-2-(2-chloro-3-fluorobenzylidene)hydrazine-1-carboxylate. ¹HNMR (400 MHz, DMSO-d6) δ 11.24 (s, 1H), 8.39 (s, 1H), 7.72 (dd, J=5.8, 3.6 Hz, 1H), 7.49-7.35 (m, 2H), 1.47 (s, 9H).; LCMS: 273.0 [M+H]⁺.

Step-b: Synthesis of tert-butyl 2-(2-chloro-3-fluorobenzyl) hydrazine-1-carboxylate

To a stirred solution of tert-butyl (E)-2-(2-chloro-3-fluorobenzyl)diazene-1 -carboxylate (5.0 g, 18.335 mmol) in 20 mL of methanol, was added acetic acid (20 mL). The reaction mixture was cooled to 0° C. To this reaction mixture, was added sodium cyanoborohydride (1.152 g, 18.33 mmol). The reaction mixture was stirred for 3 hours at room temperature under nitrogen atmosphere. The reaction mixture was distilled out and the residue obtained was diluted with DCM. The DCM layer was washed with water and brine solution then dried over anhydrous sodium sulphate and concentrated to obtain 5.1 g of tert-butyl 2-(2-chloro-3-fluorobenzyl) hydrazine-1-carboxylate. ¹H NMR (300 MHz, DMSO-d6) δ 8.31 (s, 1H), 7.48-7.23 (m, 3H), 5.12-4.94 (m, 1H), 3.98 (d, J=4.3 Hz, 2H), 1.36 (s, 9H).; LCMS: 275.1 [M+H]⁺.

Step-c: Synthesis of (2-chloro-3-fluorobenzyl) hydrazine hydrochloride

To a stirred solution of tert-butyl 2-(2-chloro-3-fluorobenzyl) hydrazine-1-carboxylate (5.0 g, 18.199 mmol) in 50 mL of DCM, was added 4N HCl in dioxane (50 mL) at 0° C. The reaction mixture was stirred for 16 hours at room temperature. The reaction mixture was concentrated under reduced pressure to get 3.8 g (98.93%) of (2-chloro-3-fluorobenzyl) hydrazine hydrochloride. ¹H NMR (300 MHz, DMSO-d6) δ 8.44 (dt, J=17.4, 6.4, 6.4 Hz, 3H), 7.48-7.28 (m, 3H), 4.13 (s, 2H).; LCMS: 175.1 [M+H]⁺.

Step-d: Synthesis of ethyl 1-(2-chloro-3-fluorobenzyl)-5-(trifluoromethyl)-1H-pyrazole-4-carboxylate

To a stirred solution of (2-chloro-3-fluorobenzyl) hydrazine hydrochloride salt (3.6 g, 17.056 mmol) in 40 mL of EtOH, was added ethyl (Z)-2-(ethoxymethylene)-4,4,4-trifluoro-3-oxobutanoate (8.194 g, 34.113 mmol). The reaction mixture was stirred for 12 hours at 80° C. The reaction mixture was concentrated under reduced pressure. The residue obtained was purified by column chromatography (SiO₂, 5-10% EtOAc in hexane) to get 3.6 g (60.18%) of ethyl 1-(2-chloro-3-fluorobenzyl)-5-(trifluoromethyl)-1H-pyrazole-4-carboxylate. ¹H NMR (300 MHz, DMSO-d6) δ 8.19 (s, 1H), 7.49-7.31 (m, 2H), 6.72-6.61 (m, 1H), 5.75 (s, 2H), 4.28 (q, J=7.2, 7.1, 7.1 Hz, 2H), 1.28 (t, J=7.1, 7.1 Hz, 3H).; LCMS: 351.1 [M+H]⁺.

Step-e: Synthesis of 1-(2-chloro-3-fluorobenzyl)-5-(trifluoromethyl)-1H-pyrazole-4-5 carboxylic acid

To a stirred solution of ethyl 1-(2-chloro-3-fluorobenzyl)-5-(trifluoromethyl)-1H-pyrazole-4-carboxylate (2.5 g, 7.128 mmol) in water (20 mL), THF (20 mL) and ethanol (20 mL) was added lithium hydroxide mono hydrate (1.496 g, 35.64 mmol) at room temperature. The reaction mixture was stirred for 12 hours. The reaction mixture was concentrated under reduced pressure. The residue obtained was diluted with water and acidified with 2N HCl until pH 2. The aqueous layer was extracted with ethyl acetate, dried over anhydrous sodium sulphate and concentrated to get 1.5 g of (65.21%) of 1-(2-chloro-3-fluorobenzyl)-5-(trifluoromethyl)-1H-pyrazole-4-carboxylic acid. ¹H NMR (300 MHz, DMSO-d6) δ 13.26 (s, 1H), 5.77-5.70 (m, 2H), 8.13 (s, 1H), 7.48-7.31 (m, 2H), 6.71-6.59 (m, 1H).; LCMS: 323.20 [M+H]⁺.

Step-f: Synthesis of 1-(2-chloro-3-fluorobenzyl)-N-(5-cyano-6-(2H-1,2,3-triazol-2-yl) pyridin-3-yl)-5-(trifluoromethyl)-1H-pyrazole-4-carboxamide

Phosphoryl chloride (1.069 g) was added to a mixture of 1-(2-chloro-3-fluorobenzyl)-5-(trifluoromethyl)-1H-pyrazole-4-carboxylic acid (0.75 g, 2.325 mmol), 5-amino-2-(2H-1,2,3-triazol-2-yl)nicotinonitrile (0.519 g, 2.79 mmol) and pyridine (1.103 g, 13.950 mmol) in

DCM (30 mL) at room temperature. The reaction mixture was stirred for 12 hours. The reaction mixture was diluted with water and extracted with ethyl acetate. The ethyl acetate layer was washed with water and brine solution then dried over anhydrous sodium sulphate and concentrated under reduced pressure to obtain 0.65 g (56.96%) of 1-(2-chloro-3-fluorobenzyl)-N-(5-cyano-6-(2H-1,2,3-triazol-2-yl)pyridin-3-yl)-5-(trifluoromethyl)-1H-pyrazole-4-carboxamide. ¹H NMR (300 MHz, DMSO-d6) δ 11.17 (s, 1H), 9.04 (d, J=2.6 Hz, 1H), 8.82 (d, J=2.5 Hz, 1H), 8.30 (d, J=5.6 Hz, 2H), 7.43 (qd, J=8.4, 8.3, 8.3, 6.0 Hz, 2H), 6.78 (dd, J=6.5, 2.3 Hz, 1H), 5.77 (s, 2H).; LCMS: 491.3 [M+H]⁺.

### Example-2

### N-(5-cyano-6-(2H-1,2,3-triazol-2-yl)pyridin-3-yl)-1-(1-phenylethyl)-5-(trifluoromethyl)-1H-pyrazole-4-carboxamide

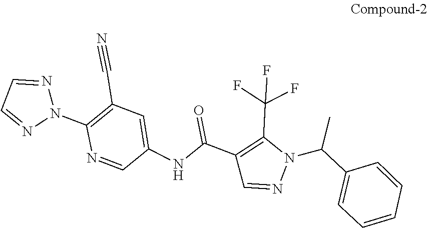 [SMILES: CC(c1ccccc1)n1ncc(C(=O)Nc2cnc(-n3nccn3)c(C#N)c2)c1C(F)(F)F]

Example-2 was prepared according to the procedure described for Example-1 with appropriate variations in reactants, quantities of reagents, solvents and reaction conditions.

The crude compound was purified by flash column chromatography over silica gel. (Gradient elution; 50-60% EtOAc in hexane) followed by preparative high-performance liquid chromatography. [Column: Regis whelk-01, 250 mm×21.1 mm×5 microns; Condition: A: n-hexane, B: EtOH: MeOH (1:1), Isocratic: 60(A):60(B), Flow rate: 15 mL/min]. The pure fraction was collected and concentrated under reduced pressure to get 0.26 g (54.48%) of N-(5-cyano-6-(2H-1,2,3-triazol-2-yl)pyridin-3-yl)-1-(1-phenyl ethyl)-5-(trifluoromethyl)-1H-pyrazole-4-carboxamide. ¹H NMR (300 MHz, methanol-d₄) δ 8.96 (d, J=2.6 Hz, 1H), 8.81 (d, J=2.6 Hz, 1H), 8.10 (s, 2H), 8.05 (s, 1H), 7.36-7.24 (m, 5H), 5.83 (q, J=6.9, 6.9, 6.9 Hz, 1H), 1.95 (d, J=6.9 Hz, 3H).; LCMS: 453.10 [M+H]⁺.

The racemic compound (0.2 g) was separated by SFC. [Column: CHIRALPAK IH (250 mm×20 mm×5 micron), Condition: solvent, A: n-hexane, B: 0.1% HCOOH in EtOH: MeOH (1:1), Isocratic: 85(A):15(B), flow rate: 15 mL/min. The pure fractions of peak-1 and peak-2 were concentrated under reduced pressure. The residues obtained were diluted with ethyl acetate. The organic layer was washed with sat. NaHCO₃ solution, water and brine solution, dried over anhydrous sodium sulphate, filtered and concentrated under reduced pressure to get peak-1(Isomer-1; Example-2a) 0.05 g (25.0%). ¹H NMR (400 MHz, DMSO-d₆) δ 11.21 (s, 1H), 9.00 (d, J=2.6 Hz, 1H), 8.79 (d, J=2.5 Hz, 1H), 8.27 (d, J=27.3 Hz, 3H), 7.46-7.19 (m, 5H), 5.88 (q, J=6.9, 6.9, 6.9 Hz, 1H), 1.90 (d, J=6.7 Hz, 3H).; LCMS: 453.2 [M+H]⁺, and peak-2 (Isomer-2; and Example-2b) 0.05 g (25.0%). ¹H NMR (400 MHz, DMSO-d₆) δ 11.24 (s, 1H), 9.01 (d, J=2.5 Hz, 1H), 8.79 (d, J=2.6 Hz, 1H), 8.27 (d, J=25.8 Hz, 3H), 7.48-7.16 (m, 5H), 5.88 (q, J=6.9, 6.8, 6.8 Hz, 1H), 1.90 (d, J=6.7 Hz, 3H).; LCMS: 453.10 [M+H]⁺.

### Example-3

### N-(5-cyano-6-(2H-1,2,3-triazol-2-yl)pyridin-3-yl)-1-(1-(3-fluorophenyl)ethyl)-5-(trifluoromethyl)-1H-pyrazole-4-carboxamide

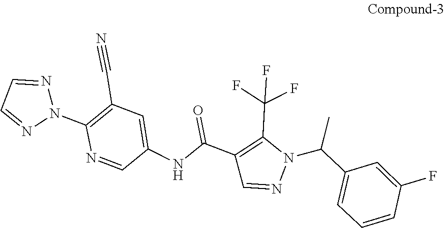 [SMILES: CC(c1cccc(F)c1)n1ncc(C(=O)Nc2cnc(-n3nccn3)c(C#N)c2)c1C(F)(F)F]

Example-3 was prepared according to the procedure described in Example-1 with appropriate variations in reactants, quantities of reagents, solvents and reaction conditions.

The crude compound was purified by flash column chromatography over silica gel. (Gradient elution; 50-60% EtOAc in hexane) followed by preparative high-performance liquid chromatography. [Column: JUPITER, 250 mm×21.2 mm×5.0 micron; Condition: A: water, B: acetonitrile, Flow rate: 20 mL/min]. The pure fraction was collected and concentrated under reduced pressure to get 0.09 g (19.27%) of N-(5-cyano-6-(2H-1,2,3-triazol-2-yl) pyridin-3-yl)-1-(1-(3-fluorophenypethyl)-5-(trifluoromethyl)-1H-pyrazole-4-carboxamide. ¹H NMR (300 MHz, DMSO-d₆) δ 11.21 (d, J=4.6 Hz, 1H), 8.99 (d, J=2.5 Hz, 1H), 8.77 (d, J=2.6 Hz, 1H), 8.29 (s, 2H), 8.24 (s, 1H), 7.41 (td, J=8.3, 8.0, 6.0 Hz, 2H), 7.12 (ddd, J=17.4, 8.7, 7.1 Hz, 2H), 5.92 (q, J=6.8, 6.7, 6.7 Hz, 1H), 1.88 (d, J=6.8 Hz, 3H).; LCMS: 471.2 [M+H]⁺.

The racemic compound (0.02 g) was separated by SFC. [Column: CHIRALPAK IH (250 mm×20 mm×5 micron), Condition: Solvent, A: n-hexane, B: EtOH, Isocratic: 80(A):20(B), Flow rate: 15 mL/min. The pure fractions of peak-1 and peak-2 were concentrated under reduced pressure to get peak-1 (Isomer-1; Example-3a) 0.008 g (26.57%). ¹H NMR (300 MHz, methanol-d₄) δ 8.97 (d, J=2.6 Hz, 1H), 8.82 (d, J=2.6 Hz, 1H), 8.10 (d, J=11.2 Hz, 3H), 7.44-7.28 (m, 1H), 7.21-6.96 (m, 3H), 5.88 (q, J=6.9, 6.9, 6.9 Hz, 1H), 1.96 (d, J=6.8 Hz, 3H).; LCMS: 471.0 [M+H]⁺, and peak-2 (Isomer-2; Example-3b) 0.008 g (26.57%). ¹H NMR (300 MHz, methanol-d₄) δ 8.98 (d, J=2.6 Hz, 1H), 8.82 (d, J=2.6 Hz, 1H), 8.10 (d, J=11.4 Hz, 3H), 7.42-7.30 (m, 1H), 7.17-6.97 (m, 3H), 5.88 (q, J=6.8, 6.8, 6.8 Hz, 1H), 1.96 (d, J=6.9 Hz, 3H).; LCMS: 471.0 [M+H]⁺.

### Example-4

### N-(5-chloro-6-(2H-1,2,3-triazol-2-yl)pyridin-3-yl)-1-(1-(3-fluorophenyl)ethyl)-5-(trifluoromethyl)-1H-pyrazole-4-carboxamide

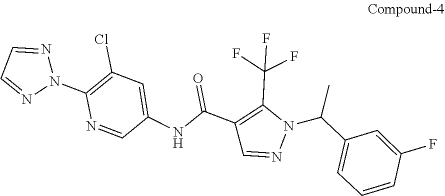 [SMILES: CC(c1cccc(F)c1)n1ncc(C(=O)Nc2cnc(-n3nccn3)c(Cl)c2)c1C(F)(F)F]

Example-4 was prepared according to the procedure described in Example-1 with appropriate variations in reactants, quantities of reagents, solvents and reaction conditions.

The crude compound was purified by flash column chromatography over silica gel. (gradient elution; 40-50% EtOAc in hexane). The pure fraction was collected and concentrated under reduced pressure to get 0.15 g (47.22%) of N-(5-chloro-6-(2H-1,2,3-triazol-2-yl) pyridin-3-yl)-1-(1-(3-fluorophenypethyl)-5-(trifluoromethyl)-1H-pyrazole-4-carboxamide. ¹H NMR (300 MHz, DMSO-d₆) δ 11.15 (s, 1H), 8.75 (d, J=2.3 Hz, 1H), 8.57 (d, J=2.3 Hz, 1H), 8.24 (s, 1H), 8.16 (s, 2H), 7.41 (td, J=8.3, 8.1, 6.1 Hz, 1H), 7.21-7.01 (m, 3H), 5.91 (q, J=6.9, 6.8, 6.8 Hz, 1H), 1.87 (d, J=6.8 Hz, 3H).; LCMS: 480.1 [M+H]⁺.

The racemic compound (0.1 g) was separated by SFC. [Column: LUX CELLULOSE-4 (250 mm×21.2 mm×5 micron), Condition: solvent, A: n-hexane, B: 0.1% DEA in EtOH, Isocratic: 85(A):15(B), Flow rate: 15 mL/min. The pure fractions of peak-1 and peak-2 were concentrated under reduced pressure to get peak-1 (Isomer-1; Example-4a) 0.045 g (45.09%). ¹H NMR (400 MHz, DMSO-d₆) δ 11.19 (s, 1H), 8.76 (d, J=2.3 Hz, 1H), 8.59 (d, J=2.1 Hz, 1H), 8.25 (s, 1H), 8.18 (s, 2H), 7.43 (q, J=7.6, 7.6, 7.5 Hz, 1H), 7.21-7.03 (m, 3H), 5.93 (q, J=6.9, 6.8, 6.8 Hz, 1H), 1.89 (d, J=6.7 Hz, 3H).; LCMS: 479.9 [M+H]⁺, and Peak-2 (Isomer-2; Example-4b) 0.045 g (45.09%). ¹H NMR (400 MHz, DMSO-d₆) δ 11.19 (s, 1H), 8.76 (d, J=2.3 Hz, 1H), 8.59 (d, J=2.1 Hz, 1H), 8.25 (s, 1H), 8.18 (s, 2H), 7.43 (q, J=7.6, 7.6, 7.5 Hz, 1H), 7.21-7.03 (m, 3H), 5.93 (q, J=6.9, 6.8, 6.8 Hz, 1H), 1.89 (d, J=6.7 Hz, 3H).; LCMS: 479.9 [M+H]⁺.

### Example-5

### N-(5-chloro-6-(2H-1,2,3-triazol-2-yl)pyridin-3-yl)-1-(1-phenylethyl)-5-(trifluoromethyl)-1H-pyrazole-4-carboxamide

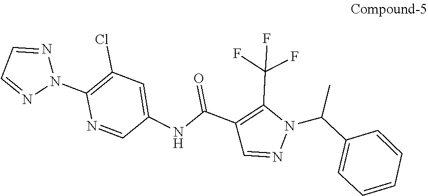 [SMILES: CC(c1ccccc1)n1ncc(C(=O)Nc2cnc(-n3nccn3)c(Cl)c2)c1C(F)(F)F]

Example-5 was prepared according to the procedure described in Example-1.

The crude compound was purified by flash column chromatography over silica gel. (gradient elution; 30-40% EtOAc in hexane). The pure fraction was collected and concentrated under reduced pressure to get 0.26 g (53.36%) of N-(5-chloro-6-(2H-1,2,3-triazol-2-yl) pyridin-3-yl)-1-(1-phenylethyl)-5-(trifluoromethyl)-1H-pyrazole-4-carboxamide. ¹HNMR (400 MHz, DMSO-d₆) δ 11.16 (s, 1H), 8.77 (d, J=2.3 Hz, 1H), 8.59 (d, J=2.3 Hz, 1H), 8.20 (d, J=22.7 Hz, 3H), 7.46-7.19 (m, 5H), 5.88 (q, J=6.8, 6.8, 6.8 Hz, 1H), 1.90 (d, J=6.8 Hz, 3H); LCMS: 462.1 [M+H]⁺.

The racemic compound (0.22 g) was separated by Supercritical Fluid Chromatography (SFC). [Column: LUX CELLULOSE-4 (250 mm×21.1 mm×5 micron), Condition: Solvent, A: n-hexane, B: 0.1% DEA in EtOH:MeOH (1:1), Isocratic: 75(A):25(B), Flow rate: 15 mL/min. The pure fractions of peak-1 and peak-2 were concentrated under reduced pressure to get peak-1 (Isomer-1; Example-5a) 0.095 g (43.21%). ¹H NMR (300 MHz, DMSO-d₆) δ 11.17 (s, 1H), 8.69 (d, J=2.3 Hz, 1H), 8.60 (d, J=2.2 Hz, 1H), 8.17 (d, J=7.9 Hz, 3H), 7.50-7.10 (m, 5H), 5.87 (q, J=6.8, 6.7, 6.7 Hz, 1H), 1.89 (d, J=6.8 Hz, 3H).; LCMS: 462.2 [M+H]⁺, and peak-2 (Isomer-2; Example-5b) 0.095 g (43.21%). ¹H NMR (300 MHz, DMSO-d₆) δ 11.17 (s, 1H), 8.76 (d, J=2.3 Hz, 1H), 8.59 (d, J=2.3 Hz, 1H), 8.20 (d, J=16.0 Hz, 3H), 7.48-7.17 (m, 5H), 5.88 (q, J=6.8, 6.8, 6.8 Hz, 1H), 1.89 (d, J=6.7 Hz, 3H).; LCMS: 462.2 [M+H]⁺.

The following compounds 6 to 56 were prepared according to the procedure described in Example-1 with appropriate variations in reactants, quantities of reagents, solvents and reaction conditions. The physiochemical characteristics of the compounds are summarized in table given below.

| Compound | Structure | Analytical data |
|---|---|---|
| 6 | 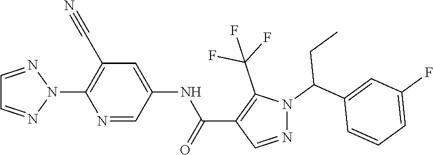 [SMILES: CCC(c1cccc(F)c1)n1ncc(C(=O)Nc2cnc(-n3nccn3)c(C#N)c2)c1C(F)(F)F] | ¹H NMR (300 MHz, DMSO-d₆) δ 11.22 (s, 1H), 8.98 (d, J = 2.5 Hz, 1H), 8.77 (d, J = 2.6 Hz, 1H), 8.27 (d, J = 8.7 Hz, 3H), 7.42 (td, J = 8.2, 8.1, 6.1 Hz, 1H), 7.28- 7.05 (m, 3H), 5.58 (dd, J = 9.7, 5.2 Hz, 1H), 2.19-2.10 (m, 1H), 2.03-1.90 (m, 1H), 0.82 (t, J = 7.1, 7.1 Hz, 3H).; LCMS: 485.1 [M + H]⁺. |
| | N-(5-cyano-6-(2H-1,2,3-triazol-2- | |
| | yl)pyridin-3-y1)-1-(1-(3- | |
| | fluorophenyl)propyl)-5- | |
| | (trifluoromethyl)-1H-pyrazole-4- | |
| | carboxamide | |
| 7 | 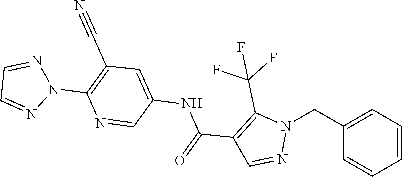 [SMILES: N#Cc1cc(NC(=O)c2cnn(Cc3ccccc3)c2C(F)(F)F)cnc1-n1nccn1] | ¹H NMR (300 MHz, Methanol-d₄) δ 9.01-8.98 (m, 1H), 8.85-8.82 (m, 1H), 8.15-8.06 (m, 3H), 7.39-7.29 (m, 3H), 7.22 (dd, J = 7.9, 5.9 Hz, 2H), 5.61 (s, 2H).; LCMS: 439.10 [M + H]⁺. |
| | 1-benzyl-N-(5-cyano-6-(2H-1,2,3- | |
| | triazol-2-yl)pyridin-3-y1)-5- | |
| | (trifluoromethyl)-1H-pyrazole-4- | |
| | carboxamide | |
| 8 | 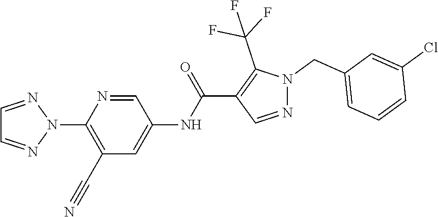 [SMILES: N#Cc1cc(NC(=O)c2cnn(Cc3cccc(Cl)c3)c2C(F)(F)F)cnc1-n1nccn1] | ¹H NMR (300 MHz, Methanol-d₄) δ 8.99 (d, J = 2.5 Hz, 1H), 8.83 (dd, J = 4.1, 2.4 Hz, 1H), 8.12 (s, 3H), 7.42-7.27 (m, 2H), 7.26-7.04 (m, 2H), 5.61 (s, 2H).; LCMS: 473.10 [M + H]⁺. |
| | 1-(3-chlorobenzyl)-N-(5-cyano-6- | |
| | (2H-1,2,3-triazol-2-y1)pyridin-3- | |
| | yl)-5-(trifluoromethyl)-1H- | |
| | pyrazole-4-carboxamide | |
| 9 | 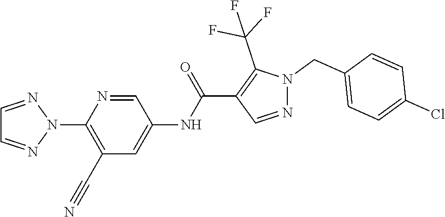 [SMILES: N#Cc1cc(NC(=O)c2cnn(Cc3ccc(Cl)cc3)c2C(F)(F)F)cnc1-n1nccn1] | ¹H NMR (400 MHz, Methanol-d₄) δ 9.01 (d, J = 2.6 Hz, 1H), 8.85 (dd, J = 5.2, 2.5 Hz, 1H), 8.13 (d, J = 7.4 Hz, 3H), 7.48- 7.35 (m, 2H), 7.24 (d, J = 8.4 Hz, 2H), 5.62 (s, 2H); LCMS: 473.05 [M + H]⁺. |
| | 1-(4-chlorobenzyl)-N-(5-cyano-6- | |
| | (2H-1,2,3-triazol-2-y1)pyridin-3- | |
| | yl)-5-(trifluoromethyl)-1H- | |
| | pyrazole-4-carboxamide | |
| 10 | 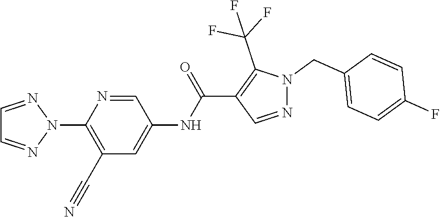 [SMILES: N#Cc1cc(NC(=O)c2cnn(Cc3ccc(F)cc3)c2C(F)(F)F)cnc1-n1nccn1] | ¹H NMR (300 MHz, Methanol-d₄) δ 8.99 (d, J = 2.5 Hz, 1H), 8.83 (d, J = 2.6 Hz, 1H), 8.10 (d, J = 8.1 Hz, 3H), 7.28 (dd, J = 8.4, 5.3 Hz, 2H), 7.09 (t, J = 8.7, 8.7 Hz, 2H), 5.58 (s, 2H); LCMS: 457.05 [M + H]⁺. |
| | N-(5-cyano-6-(2H-1,2,3-triazol-2- | |
| | yl)pyridin-3-yl)-1-(4-fluorobenzyl)- | |
| | 5-(trifluoromethyl)-1H-pyrazole-4- | |
| | carboxamide | |
| 11 | 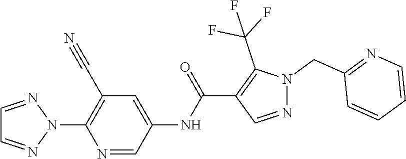 [SMILES: N#Cc1cc(NC(=O)c2cnn(Cc3ccccn3)c2C(F)(F)F)cnc1-n1nccn1] | ¹H NMR (300 MHz, Methanol-d₄) δ 8.98 (d, J = 2.6 Hz, 1H), 8.83 (d, J = 2.6 Hz, 1H), 8.59 - 8.48 (m, 2H), 8.10 (s, 2H), 7.88 (td, J = 7.8, 7.8, 1.8 Hz, 1H), 7.46- 7.35 (m, 2H), 5.57 (s, 2H).; LCMS: 440.1 [M + H]⁺. |
| | N-(5-cyano-6-(2H-1,2,3-triazol-2- | |
| | yl)pyridin-3-y1)-1-(pyridin-2- | |
| | ylmethyl)-5-(trifluoromethyl)-1H- | |
| | pyrazole-4-carboxamide | |
| 12 | 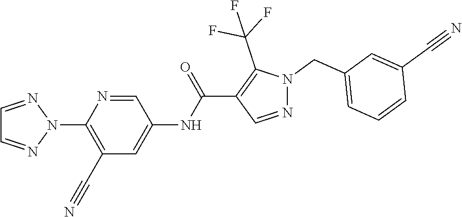 [SMILES: N#Cc1cccc(Cn2ncc(C(=O)Nc3cnc(-n4nccn4)c(C#N)c3)c2C(F)(F)F)c1] | ¹H NMR (400 MHz, DMSO-d₆) δ 11.17 (s, 1H), 9.03 (d, J = 2.5 Hz, 1H), 8.81 (d, J = 2.5 Hz, 1H), 8.30 (d, J = 2.2 Hz, 3H), 7.84 (d, J = 7.8 Hz, 1H), 7.71-7.42 (m, 3H), 5.73 (s, 2H); LCMS: 464.1 [M + H]⁺. |
| | N-(5-cyano-6-(2H-1,2,3-triazol-2- | |
| | yl)pyridin-3-y1)-1-(3-cyanobenzyl)- | |
| | 5-(trifluoromethyl)-1H-pyrazole-4- | |
| | carboxamide | |
| 13 | 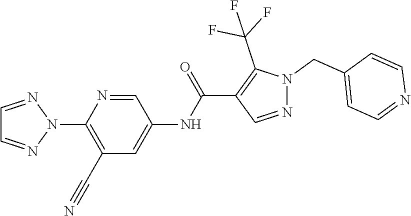 [SMILES: N#Cc1cc(NC(=O)c2cnn(Cc3ccncc3)c2C(F)(F)F)cnc1-n1nccn1] | ¹H NMR (400 MHz, Methanol-d₄) δ 9.02 (d, J = 2.6 Hz, 1H), 8.85 (dd, J = 4.8, 2.5 Hz, 1H), 8.56 (s, 2H), 8.16 (d, J = 17.1 Hz, 3H), 7.23 (d, J = 5.1 Hz, 2H), 5.73 (s, 2H); LCMS: 440.0 [M + H]⁺. |
| | N-(5-cyano-6-(2H-1,2,3-triazol-2- | |
| | yl)pyridin-3-yl)-1-(pyridin-4- | |
| | ylmethyl)-5-(trifluoromethyl)-1H- | |
| | pyrazole-4-carboxamide | |
| 14 | 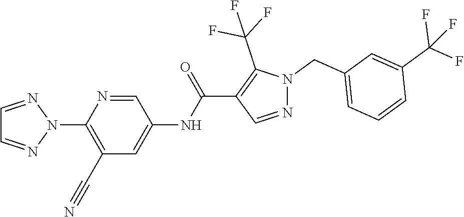 [SMILES: N#Cc1cc(NC(=O)c2cnn(Cc3cccc(C(F)(F)F)c3)c2C(F)(F)F)cnc1-n1nccn1] | ¹H NMR (400 MHz, DMSO-d₆) δ 11.17 (s, 1H), 9.03 (d, J = 2.6 Hz, 1H), 8.81 (d, J = 2.6 Hz, 1H), 8.30 (d, J = 1.5 Hz, 3H), 7.82-7.35 (m, 4H), 5.77 (s, 2H).; LCMS: 506.70 [M + H]⁺. |
| | N-(5-cyano-6-(2H-1,2,3-triazol-2- | |
| | yl)pyridin-3-y1)-5- | |
| | (trifluoromethyl)-1-(3- | |
| | (trifluoromethyl)benzyl)-1H- | |
| | pyrazole-4-carboxamide | |
| 15 | 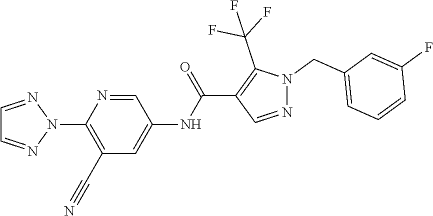 [SMILES: N#Cc1cc(NC(=O)c2cnn(Cc3cccc(F)c3)c2C(F)(F)F)cnc1-n1nccn1] | ¹H NMR (400 MHz, DMSO-d₆) δ 11.16 (s, 1H), 9.03 (d, J = 2.5 Hz, 1H), 8.81 (d, J = 2.5 Hz, 1H), 8.30 (d, J = 4.9 Hz, 3H), 7.44 (td, J = 8.0, 7.9, 6.0 Hz, 1H), 7.19 (td, J = 8.8, 8.7, 2.5 Hz, 1H), 7.00 (t, J = 7.7, 7.7 Hz, 2H), 5.67 (s, 2H).; LCMS: 456.65 [M + H]⁺. |
| | N-(5-cyano-6-(2H-1,2,3-triazol-2- | |
| | yl)pyridin-3-y1)-1-(3-fluorobenzy1)- | |
| | 5-(trifluoromethyl)-1H-pyrazole-4- | |
| | carboxamide | |
| 16 | 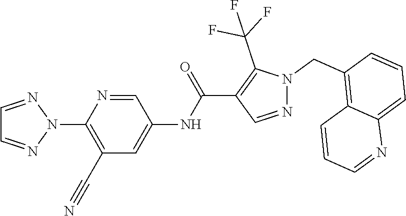 [SMILES: N#Cc1cc(NC(=O)c2cnn(Cc3cccc4ncccc34)c2C(F)(F)F)cnc1-n1nccn1] | ¹H NMR (400 MHz, Methanol-d₄) δ 8.97-8.86 (m, 2H), 8.78 (d, J = 2.5 Hz, 1H), 8.68 (d, J = 8.6 Hz, 2H), 8.21-8.00 (m, 3H), 7.92-7.82 (m, 1H), 7.73 (m, J = 7.1 Hz, 2H), 6.03 (s, 2H).; LCMS: 490.05 [M + H]⁺. |
| | N-(5-cyano-6-(2H-1,2,3-triazol-2- | |
| | yl)pyridin-3-y1)-1-(quinolin-5- | |
| | ylmethyl)-5-(trifluoromethyl)-1H- | |
| | pyrazole-4-carboxamide | |
| 17 | 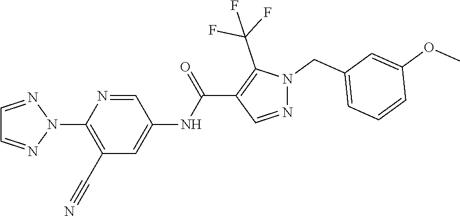 [SMILES: COc1cccc(Cn2ncc(C(=O)Nc3cnc(-n4nccn4)c(C#N)c3)c2C(F)(F)F)c1] | ¹H NMR (400 MHz, Methanol-d₄) δ 8.97 (d, J = 2.6 Hz, 1H), 8.82 (d, J = 2.6 Hz, 1H), 8.41 (s, 1H), 8.12 (s, 2H), 7.34 (dd, J = 8.8, 6.8 Hz, 1H), 7.03-6.85 (m, 3H), 5.44 (s, 2H), 3.81 (s, 3H).; LCMS: 469.1 [M + H]⁺. |
| | N-(5-cyano-6-(2H-1,2,3-triazol-2- | |
| | yl)pyridin-3-y1)-1-(3- | |
| | methoxybenzyl)-5- | |
| | (trifluoromethyl)-1H-pyrazole-4- | |
| | carboxamide | |
| 18 | 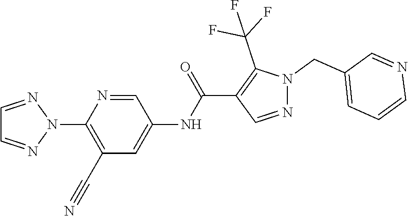 [SMILES: N#Cc1cc(NC(=O)c2cnn(Cc3cccnc3)c2C(F)(F)F)cnc1-n1nccn1] | ¹H NMR (400 MHz, DMSO-d₆) δ 11.16 (s, 1H), 9.02 (d, J = 2.6 Hz, 1H), 8.80 (d, J = 2.5 Hz, 1H), 8.61 - 8.45 (m, 2H), 8.29 (d, J = 11.4 Hz, 3H), 7.61 (dt, J = 7.9, 2.0, 2.0 Hz, 1H), 7.43 (dd, J = 7.9, 4.7 Hz, 1H), 5.70 (s, 2H).; LCMS: 439.9 [M + H]⁺. |
| | N-(5-cyano-6-(2H-1,2,3-triazol-2- | |
| | yl)pyridin-3-yl)-1-(pyridin-3- | |
| | ylmethyl)-5-(trifluoromethyl)-1H- | |
| | pyrazole-4-carboxamide | |
| 19 | 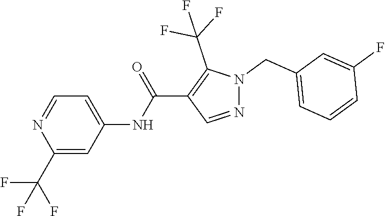 [SMILES: O=C(Nc1ccnc(C(F)(F)F)c1)c1cnn(Cc2cccc(F)c2)c1C(F)(F)F] | ¹H NMR (400 MHz, Methanol-d₄) δ 8.61 (d, J = 5.6 Hz, 1H), 8.19 (d, J = 2.1 Hz, 1H), 8.11 (s, 1H), 7.92 (dd, J = 5.7, 2.0 Hz, 1H), 7.39 (td, J = 8.0, 8.0, 5.9 Hz, 1H), 7.18-7.01 (m, 2H), 6.95 (dd, J = 9.8, 2.3 Hz, 1H), 5.63 (s, 2H).; LCMS: 433.0 [M + H]⁺. |
| | 1-(3-fluorobenzyl)-5- | |
| | (trifluoromethyl)-N-(2- | |
| | (trifluoromethyl)pyridin-4-yl)-1H- | |
| | pyrazole-4-carboxamide | |
| 20 | 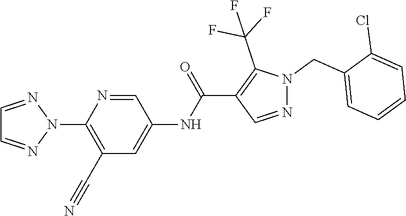 [SMILES: N#Cc1cc(NC(=O)c2cnn(Cc3ccccc3Cl)c2C(F)(F)F)cnc1-n1nccn1] | ¹H NMR (300 MHz, Methanol-d₄) δ 9.01 (d, J = 2.7 Hz, 1H), 8.85 (d, J = 2.6 Hz, 1H), 8.12 (s, 2H), 7.50-7.45 (m, 1H), 7.37-7.23 (m, 2H), 6.79 (d, J = 7.9 Hz, 2H), 5.72 (s, 2H).; LCMS: 473.05 [M + H]⁺. |
| | 1-(2-chlorobenzyl)-N-(5-cyano-6- | |
| | (2H-1,2,3-triazol-2-y1)pyridin-3- | |
| | yl)-5-(trifluoromethyl)-1H- | |
| | pyrazole-4-carboxamide | |
| 21 | 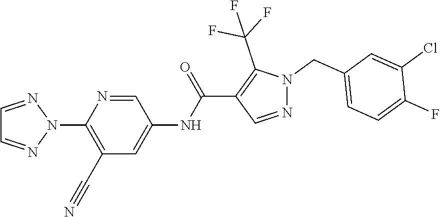 [SMILES: N#Cc1cc(NC(=O)c2cnn(Cc3ccc(F)c(Cl)c3)c2C(F)(F)F)cnc1-n1nccn1] | ¹H NMR (300 MHz, Methanol-d₄) δ 8.95 (d, J = 2.6 Hz, 1H), 8.80 (d, J = 2.6 Hz, 1H), 8.43 (s, 1H), 8.10 (s, 2H), 7.56 (dd, J = 7.0, 2.2 Hz, 1H), 7.45-7.20 (m, 2H), 5.45 (s, 2H).; LCMS: 490.9 [M + H]⁺. |
| | 1-(3-chloro-4-fluorobenzy1)-N-(5- | |
| | cyano-6-(2H-1,2,3-triazol-2- | |
| | yl)pyridin-3-yl)-5- | |
| | (trifluoromethyl)-1H-pyrazole-4- | |
| | carboxamide | |
| 22 | 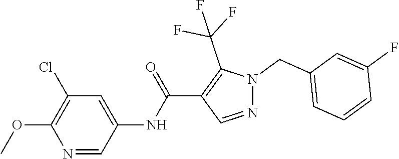 [SMILES: COc1ncc(NC(=O)c2cnn(Cc3cccc(F)c3)c2C(F)(F)F)cc1Cl] | ¹H NMR (300 MHz, Methanol-d₄) δ 8.29 (d, J = 2.3 Hz, 1H), 8.14 (d, J = 2.5 Hz, 1H), 8.01 (s, 1H), 7.52-7.25 (m, 1H), 7.14-6.84 (m, 3H), 5.59 (s, 2H), 3.97 (d, J = 2.1 Hz, 3H).; LCMS: 428.8 [M + H]⁺. |
| | N-(5-chloro-6-methoxypyridin-3- | |
| | yl)-1-(3-fluorobenzyl)-5- | |
| | (trifluoromethyl)-1H-pyrazole-4- | |
| | carboxamide | |
| 23 | 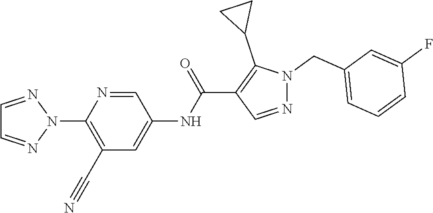 [SMILES: N#Cc1cc(NC(=O)c2cnn(Cc3cccc(F)c3)c2C2CC2)cnc1-n1nccn1] | ¹H NMR (300 MHz, Methanol-d₄) δ 9.01 (d, J = 2.5 Hz, 1H), 8.86 (t, J = 2.4, 2.4 Hz, 1H), 8.11 (s, 2H), 8.00 (s, 1H), 7.36 (td, J = 8.0, 8.0, 5.9 Hz, 1H), 7.09-6.84 (m, 3H), 5.55 (s, 2H), 1.85-1.76 (m, 1H), 1.13-1.03 (m, 2H), 0.81-0.69 (m, 2H).; LCMS: 429.05 [M + H]⁺. |
| | N-(5-cyano-6-(2H-1,2,3-triazol-2- | |
| | yl)pyridin-3-yl)-5-cyclopropyl-1- | |
| | (3-fluorobenzyl)-1H-pyrazole-4- | |
| | carboxamide | |
| 24 | 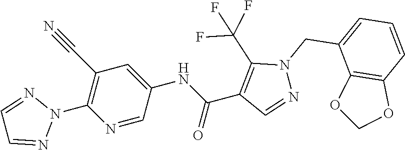 [SMILES: N#Cc1cc(NC(=O)c2cnn(Cc3cccc4c3OCO4)c2C(F)(F)F)cnc1-n1nccn1] | ¹H NMR (300 MHz, Methanol-d₄) δ 8.87-8.78 (m, 1H), 8.42-8.37 (m, 1H), 8.19-8.05 (m, 3H), 6.87 (d, J = 1.4 Hz, 3H), 6.08 (s, 2H), 5.43 (s, 2H).; LCMS: 483.1 [M + H]⁺. |
| | 1-(benzo[d][1,3]dioxol-4- | |
| | ylmethyl)-N-(5-cyano-6-(2H-1,2,3- | |
| | triazol-2-y1)pyridin-3-y1)-5- | |
| | (trifluoromethyl)-1H-pyrazole-4- | |
| | carboxamide | |
| 25 | 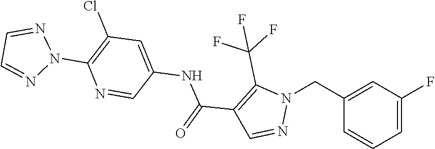 [SMILES: O=C(Nc1cnc(-n2nccn2)c(Cl)c1)c1cnn(Cc2cccc(F)c2)c1C(F)(F)F] | ¹H NMR (400 MHz, DMSO-d₆) δ 11.11 (s, 1H), 8.79 (d, J = 2.3 Hz, 1H), 8.61 (d, J = 2.2 Hz, 1H), 8.29 (s, 1H), 8.18 (d, J = 1.3 Hz, 2H), 7.44 (td, J = 8.2, 8.1, 6.1 Hz, 1H), 7.23-7.14 (m, 1H), 7.00 (t, J = 7.7, 7.7 Hz, 2H), 5.67 (s, 2H).; LCMS: 465.0 [M + H]⁺. |
| | N-(5-chloro-6-(2H-1,2,3-triazol-2- | |
| | yl)pyridin-3-yl)-1-(3-fluorobenzyl)- | |
| | 5-(trifluoromethyl)-1H-pyrazole-4- | |
| | carboxamide | |
| 26 | 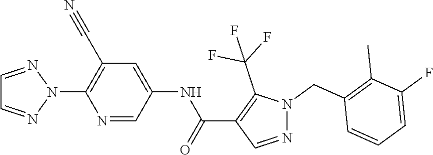 [SMILES: Cc1c(F)cccc1Cn1ncc(C(=O)Nc2cnc(-n3nccn3)c(C#N)c2)c1C(F)(F)F] | ¹H NMR (300 MHz, DMSO-d₆) δ 11.17 (s, 1H), 9.04 (d, J = 2.5 Hz, 1H), 8.83 (d, J = 2.5 Hz, 1H), 8.30 (d, J = 6.0 Hz, 3H), 7.35-7.01 (m, 2H), 6.43 (d, J = 7.5 Hz, 1H), 5.69 (s, 2H), 2.22 (d, J = 1.9 Hz, 3H).; LCMS: 471.0 [M + H]⁺. |
| | N-(5-cyano-6-(2H-1,2,3-triazol-2- | |
| | yl)pyridin-3-y1)-1-(3-fluoro-2- | |
| | methylbenzyl)-5-(trifluoromethyl)- | |
| | 1H-pyrazole-4-carboxamide | |
| 27 | 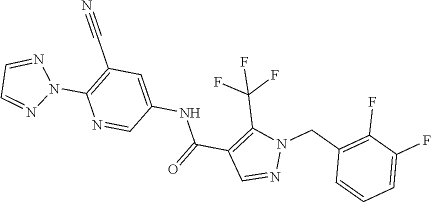 [SMILES: N#Cc1cc(NC(=O)c2cnn(Cc3cccc(F)c3F)c2C(F)(F)F)cnc1-n1nccn1] | ¹H NMR (300 MHz, DMSO-d₆) δ 11.16 (s, 1H), 9.02 (d, J = 2.6 Hz, 1H), 8.81 (d, J = 2.5 Hz, 1H), 8.28 (d, J = 14.4 Hz, 3H), 7.47 (dtd, J = 10.1, 8.2, 8.2, 1.6 Hz, 1H), 7.25 (tdd, J = 8.2, 8.2, 5.0, 1.7 Hz, 1H), 6.97 (tt, J = 7.8, 7.8, 1.6, 1.6 Hz, 1H), 5.74 (s, 2H).; LCMS: 475.1 [M + H]⁺. |
| | N-(5-cyano-6-(2H-1,2,3-triazol-2- | |
| | yl)pyridin-3-y1)-1-(2,3- | |
| | difluorobenzyl)-5- | |
| | (trifluoromethyl)-1H-pyrazole-4- | |
| | carboxamide | |
| 28 | 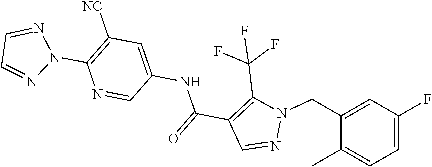 [SMILES: Cc1ccc(F)cc1Cn1ncc(C(=O)Nc2cnc(-n3nccn3)c(C#N)c2)c1C(F)(F)F] | ¹H NMR (300 MHz, Methanol-d₄) δ 9.10-8.95 (m, 1H), 8.86 (d, J = 2.6 Hz, 1H), 8.13 (d, J = 2.5 Hz, 3H), 7.24 (dd, J = 8.5, 5.7 Hz, 1H), 6.96 (td, J = 8.5, 8.5, 2.8 Hz, 1H), 6.34 (dd, J = 9.9, 2.7 Hz, 1H), 5.62 (s, 2H), 2.33 (s, 3H).; LCMS: 471.05 [M + H]⁺. |
| | N-(5-cyano-6-(2H-1,2,3-triazol-2- | |
| | yl)pyridin-3-yl)-1-(5-fluoro-2- | |
| | methylbenzyl)-5-(trifluoromethyl)- | |
| | 1H-pyrazole-4-carboxamide | |
| 29 | 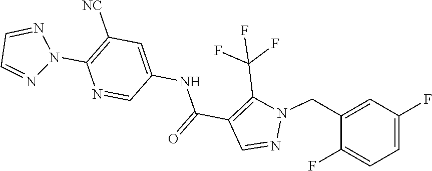 [SMILES: N#Cc1cc(NC(=O)c2cnn(Cc3cc(F)ccc3F)c2C(F)(F)F)cnc1-n1nccn1] | ¹H NMR (400 MHz, DMSO-d₆) δ 11.16 (s, 1H), 9.02 (d, J = 2.6 Hz, 1H), 8.81 (d, J = 2.6 Hz, 1H), 8.28 (d, J = 21.2 Hz, 3H), 7.45-7.12 (m, 2H), 7.00 (ddd, J = 8.9, 5.8, 3.1 Hz, 1H), 5.68 (s, 2H).; LCMS: 475.0 [M + H]⁺. |
| | N-(5-cyano-6-(2H-1,2,3-triazol-2- | |
| | yl)pyridin-3-y1)-1-(2,5- | |
| | difluorobenzyl)-5- | |
| | (trifluoromethyl)-1H-pyrazole-4- | |
| | carboxamide | |
| 30 | 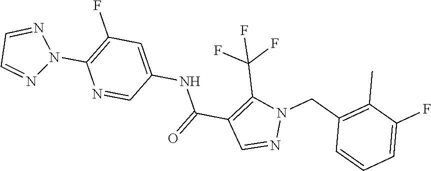 [SMILES: Cc1c(F)cccc1Cn1ncc(C(=O)Nc2cnc(-n3nccn3)c(F)c2)c1C(F)(F)F] | ¹H NMR (400 MHz, Methanol-d₄) δ 8.62 (d, J = 2.2 Hz, 1H), 8.51 (dd, J = 12.1, 2.2 Hz, 1H), 8.09 (d, J = 16.1 Hz, 3H), 7.16 (td, J = 8.0, 8.0, 5.7 Hz, 1H), 7.05 (t, J = 9.0, 9.0 Hz, 1H), 6.53 (d, J = 7.7 Hz, 1H), 5.66 (s, 2H), 2.29 (d, J = 2.0 Hz, 3H).; LCMS: 464.05 [M + H]⁺. |
| | 1-(3-fluoro-2-methylbenzyl)-N-(5- | |
| | fluoro-6-(2H-1,2,3-triazol-2- | |
| | yl)pyridin-3-y1)-5- | |
| | (trifluoromethyl)-1H-pyrazole-4- | |
| | carboxamide | |
| 31 | 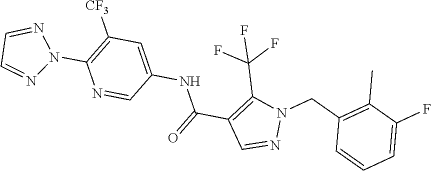 [SMILES: Cc1c(F)cccc1Cn1ncc(C(=O)Nc2cnc(-n3nccn3)c(C(F)(F)F)c2)c1C(F)(F)F] | ¹H NMR (400 MHz, Methanol-d₄) δ 9.08 (d, J = 2.4 Hz, 1H), 8.90 (d, J = 2.4 Hz, 1H), 8.15 (s, 1H), 8.05 (s, 2H), 7.26- 6.99 (m, 3H), 6.54 (d, J = 7.7 Hz, 1H), 5.67 (s, 2H), 2.29 (d, J = 1.9 Hz, 3H).; LCMS: 513.75 [M + H]⁺. |
| | N-(6-(2H-1,2,3-triazol-2-y1)-5- | |
| | (trifluoromethyl)pyridin-3-y1)-1-(3- | |
| | fluoro-2-methylbenzy1)-5- | |
| | (trifluoromethyl)-1H-pyrazole-4- | |
| | carboxamide | |
| 32 | 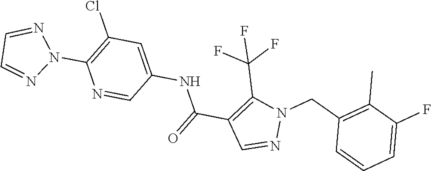 [SMILES: Cc1c(F)cccc1Cn1ncc(C(=O)Nc2cnc(-n3nccn3)c(Cl)c2)c1C(F)(F)F] | ¹H NMR (300 MHz, DMSO-d₆) δ 11.12 (s, 1H), 8.81 (d, J = 2.2 Hz, 1H), 8.63 (d, J = 2.2 Hz, 1H), 8.29 (s, 1H), 8.18 (s, 2H), 7.33-7.03 (m, 2H), 6.44 (d, J = 7.4 Hz, 1H), 5.69 (s, 2H), 2.22 (d, J = 2.0 Hz, 3H).; LCMS: 479.9 [M + H]⁺. |
| | N-(5-chloro-6-(2H-1,2,3-triazol-2- | |
| | y1)pyridin-3-y1)-1-(3-fluoro-2- | |
| | methylbenzyl)-5-(trifluoromethyl)- | |
| | 1H-pyrazole-4-carboxamide | |
| 33 | 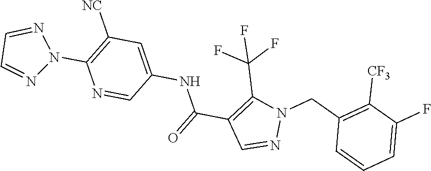 [SMILES: N#Cc1cc(NC(=O)c2cnn(Cc3cccc(F)c3C(F)(F)F)c2C(F)(F)F)cnc1-n1nccn1] | ¹H NMR (300 MHz, Methanol-d₄) δ 9.01 (d, J = 2.4 Hz, 1H), 8.86 (t, J = 2.2, 2.2 Hz, 1H), 8.14 (d, J = 8.6 Hz, 3H), 7.60 (td, J = 8.0, 8.0, 5.2 Hz, 1H), 7.39-7.28 (m, 1H), 6.57 (d, J = 7.9 Hz, 1H), 5.84 (s, 2H).; LCMS: 525.2 [M + H]⁺. |
| | N-(5-cyano-6-(2H-1,2,3-triazol-2- | |
| | yl)pyridin-3-y1)-1-(3-fluoro-2- | |
| | (trifluoromethyl)benzy1)-5- | |
| | (trifluoromethyl)-1H-pyrazole-4- | |
| | carboxamide | |
| 34 | 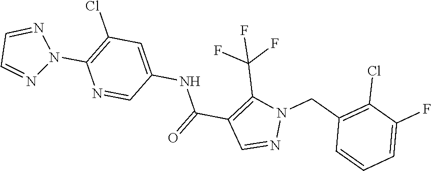 [SMILES: O=C(Nc1cnc(-n2nccn2)c(Cl)c1)c1cnn(Cc2cccc(F)c2Cl)c1C(F)(F)F] | ¹H NMR (300 MHz, DMSO-d₆) δ 11.10 (s, 1H), 8.79 (d, J = 2.2 Hz, 1H), 8.60 (d, J = 2.1 Hz, 1H), 8.27 (s, 1H), 8.17 (s, 2H), 7.41 (qd, J = 8.4, 8.3, 8.3, 6.0 Hz, 2H), 6.79-6.74 (m, 1H), 5.75 (s, 2H).; LCMS: 499.9 [M + H]⁺. |
| | 1-(2-chloro-3-fluorobenzy1)-N-(5- | |
| | chloro-6-(2H-1,2,3-triazol-2- | |
| | yl)pyridin-3-yl)-5- | |
| | (trifluoromethyl)-1H-pyrazole-4- | |
| | carboxamide | |
| 35 | 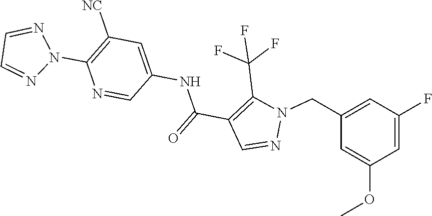 [SMILES: COc1cc(F)cc(Cn2ncc(C(=O)Nc3cnc(-n4nccn4)c(C#N)c3)c2C(F)(F)F)c1] | ¹H NMR (300 MHz, DMSO-d₆) δ 11.15 (s, 1H), 9.01 (d, J = 2.5 Hz, 1H), 8.80 (d, J = 2.6 Hz, 1H), 8.28 (d, J = 3.4 Hz, 3H), 6.81 (dt, J = 11.1, 2.3, 2.3 Hz, 1H), 6.60-6.42 (m, 2H), 5.60 (s, 2H), 3.81- 3.66 (m, 3H).; LCMS: 487.3 [M + H]⁺. |
| | N-(5-cyano-6-(2H-1,2,3-triazol-2- | |
| | yl)pyridin-3-yl)-1-(3-fluoro-5- | |
| | methoxybenzyl)-5- | |
| | (trifluoromethyl)-1H-pyrazole-4- | |
| | carboxamide | |
| 36 | 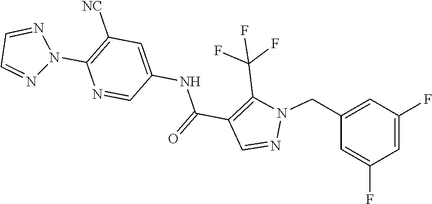 [SMILES: N#Cc1cc(NC(=O)c2cnn(Cc3cc(F)cc(F)c3)c2C(F)(F)F)cnc1-n1nccn1] | ¹H NMR (300 MHz, DMSO-d₆) δ 11.15 (s, 1H), 9.00 (d, J = 2.7 Hz, 1H), 8.80 (t, J = 3.0, 3.0 Hz, 1H), 8.29 (s, 3H), 7.23 (t, J = 9.4, 9.4 Hz, 1H), 6.86 (d, J = 7.2 Hz, 2H), 5.68 (s, 2H).; LCMS: 475.1 [M + H]⁺. |
| | N-(5-cyano-6-(2H-1,2,3-triazol-2- | |
| | y1)pyridin-3-yl)-1-(3,5- | |
| | difluorobenzyl)-5- | |
| | (trifluoromethyl)-1H-pyrazole-4- | |
| | carboxamide | |
| 37 | 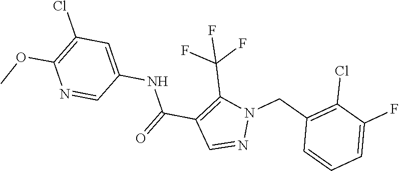 [SMILES: COc1ncc(NC(=O)c2cnn(Cc3cccc(F)c3Cl)c2C(F)(F)F)cc1Cl] | ¹H NMR (300 MHz, DMSO-d₆) δ 10.59 (s, 1H), 8.35 (d, J = 2.3 Hz, 1H), 8.29- 8.12 (m, 2H), 7.40 (qd, J = 8.6, 8.4, 8.4, 6.0 Hz, 2H), 6.79-6.66 (m, 1H), 5.71 (s, 2H), 3.91 (s, 3H).; LCMS: 464.9 [M + H]⁺. |
| | 1-(2-chloro-3-fluorobenzyl)-N-(5- | |
| | chloro-6-methoxypyridin-3-y1)-5- | |
| | (trifluoromethyl)-1H-pyrazole-4- | |
| | carboxamide | |
| 38 | 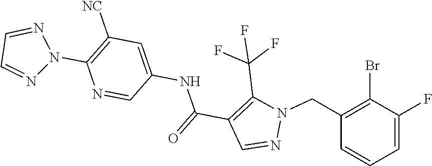 [SMILES: N#Cc1cc(NC(=O)c2cnn(Cc3cccc(F)c3Br)c2C(F)(F)F)cnc1-n1nccn1] | ¹H NMR (300 MHz, DMSO-d₆) δ 11.15 (s, 1H), 9.02 (d, J = 2.5 Hz, 1H), 8.81 (d, J = 2.6 Hz, 1H), 8.29 (d, J = 3.0 Hz, 3H), 7.42 (dtd, J = 14.7, 8.5, 8.3, 6.2 Hz, 2H), 6.72-6.58 (m, 1H), 5.72 (s, 2H).; LCMS: 536.1 [M + H]⁺. |
| | 1-(2-bromo-3-fluorobenzy1)-N-(5- | |
| | cyano-6-(2H-1,2,3-triazol-2- | |
| | yl)pyridin-3-yl)-5- | |
| | (trifluoromethyl)-1H-pyrazole-4- | |
| | carboxamide | |
| 39 | 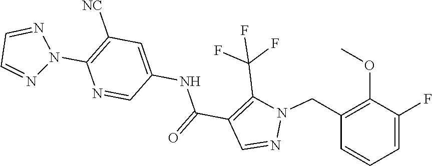 [SMILES: COc1c(F)cccc1Cn1ncc(C(=O)Nc2cnc(-n3nccn3)c(C#N)c2)c1C(F)(F)F] | ¹H NMR (300 MHz, DMSO-d₆) δ 11.13 (s, 1H), 9.02 (d, J = 2.6 Hz, 1H), 8.80 (d, J = 2.6 Hz, 1H), 8.26 (d, J = 17.5 Hz, 2H), 7.80-7.55 (m, 1H), 7.39-7.19 (m, 1H), 7.08 (td, J = 8.0, 8.0, 5.0 Hz, 1H), 6.71 (d, J = 7.7 Hz, 1H), 5.62 (s, 2H), 3.82 (d, J = 1.9 Hz, 3H).; LCMS: 487.0 [M + H]⁺. |
| | N-(5-cyano-6-(2H-1,2,3-triazol-2- | |
| | yl)pyridin-3-y1)-1-(3-fluoro-2- | |
| | methoxybenzyl)-5- | |
| | (trifluoromethyl)-1H-pyrazole-4- | |
| | carboxamide | |
| 40 | 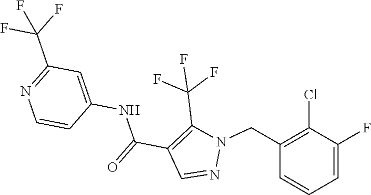 [SMILES: O=C(Nc1ccnc(C(F)(F)F)c1)c1cnn(Cc2cccc(F)c2Cl)c1C(F)(F)F] | H NMR (400 MHz, DMSO-d₆) δ 11.12 (s, 1H), 8.68 (d, J = 5.6 Hz, 1H), 8.23 (d, J = 31.8 Hz, 2H), 7.96-7.89 (m, 1H), 7.44 (dt, J = 10.4, 7.9, 7.9 Hz, 2H), 6.78 (d, J = 7.3 Hz, 1H), 5.75 (s, 2H).; LCMS: 466.95 [M + H]⁺. |
| | 1-(2-chloro-3-fluorobenzyl)-5- | |
| | (trifluoromethyl)-N-(2- | |
| | (trifluoromethyl)pyridin-4-y1)-1H- | |
| | pyrazole-4-carboxamide | |
| 41 | 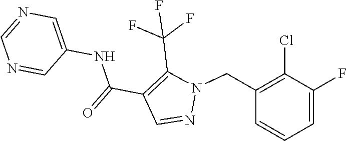 [SMILES: O=C(Nc1cncnc1)c1cnn(Cc2cccc(F)c2Cl)c1C(F)(F)F] | ¹H NMR (400 MHz, DMSO-d₆) δ 10.83 (s, 1H), 9.09 (s, 2H), 8.95 (s, 1H), 8.25 (s, 1H), 7.51-7.33 (m, 2H), 6.76 (d, J = 7.3 Hz, 1H), 5.75 (s, 2H).; LCMS: 399.95 [M + H]⁺. |
| | 1-(2-chloro-3-fluorobenzyl)-N- | |
| | (pyrimidin-5-y1)-5- | |
| | (trifluoromethyl)-1H-pyrazole-4- | |
| | carboxamide | |
| 42 | 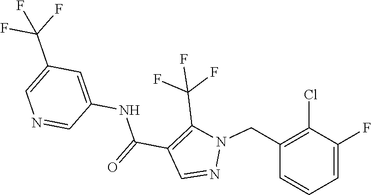 [SMILES: O=C(Nc1cncc(C(F)(F)F)c1)c1cnn(Cc2cccc(F)c2Cl)c1C(F)(F)F] | ¹H NMR (400 MHz, Methanol-d₄) δ 9.02 (d, J = 2.3 Hz, 1H), 8.75-8.51 (m, 2H), 8.11 (s, 1H), 7.43-7.11 (m, 2H), 6.66 (d, J = 7.6 Hz, 1H), 5.75 (s, 2H).; LCMS: 467.0 [M + H]⁺. |
| | 1-(2-chloro-3-fluorobenzyl)-5- | |
| | (trifluoromethyl)-N-(5- | |
| | (trifluoromethyl)pyridin-3-yl)-1H- | |
| | pyrazole-4-carboxamide | |
| 43 | 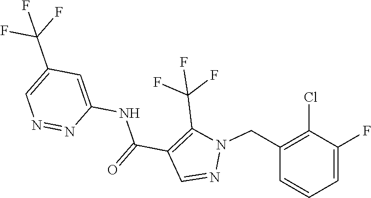 [SMILES: O=C(Nc1cc(C(F)(F)F)cnn1)c1cnn(Cc2cccc(F)c2Cl)c1C(F)(F)F] | ¹H NMR (300 MHz, DMSO-d₆) δ 12.21 (s, 1H), 9.50 (d, J = 1.9 Hz, 1H), 8.66 (d, J = 1.7 Hz, 1H), 8.36 (s, 1H), 7.43 (qd, J = 9.0, 8.4, 8.4, 6.4 Hz, 2H), 6.80-6.71 (m, 1H), 5.75 (s, 2H).; LCMS: 468.0 [M + H]⁺. |
| | 1-(2-chloro-3-fluorobenzyl)-5- | |
| | (trifluoromethyl)-N-(5- | |
| | (trifluoromethyl)pyridazin-3-y1)- | |
| | 1H-pyrazole-4-carboxamide | |
| 44 | 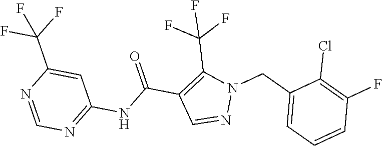 [SMILES: O=C(Nc1cc(C(F)(F)F)ncn1)c1cnn(Cc2cccc(F)c2Cl)c1C(F)(F)F] | ¹H NMR (300 MHz, DMSO-d₆) δ 12.06 (s, 1H), 9.19 (s, 1H), 8.50 (s, 1H), 8.33 (s, 1H), 7.55-7.28 (m, 2H), 6.81-6.68 (m, 1H), 5.75 (s, 2H).; LCMS: 468.10 [M + H]⁺. |
| | 1-(2-chloro-3-fluorobenzyl)-5- | |
| | (trifluoromethyl)-N-(6- | |
| | (trifluoromethyl)pyrimidin-4-yl)- | |
| | 1H-pyrazole-4-carboxamide | |
| 45 | 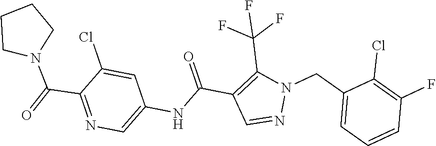 [SMILES: O=C(Nc1cnc(C(=O)N2CCCC2)c(Cl)c1)c1cnn(Cc2cccc(F)c2Cl)c1C(F)(F)F] | ¹H NMR (300 MHz, methanol-d₄) δ 8.74 (d, J = 2.1 Hz, 1H), 8.47 (d, J = 2.1 Hz, 1H), 8.09 (s, 1H), 7.39-7.14 (m, 2H), 6.65 (d, J = 7.4 Hz, 1H), 5.74 (s, 2H), 3.63 (t, J = 6.7, 6.7 Hz, 2H), 1.98 (td, J = 12.5, 12.4, 6.4 Hz, 4H), 1.29 (s, 2H).; LCMS: 531.1 [M + H]⁺. |
| | 1-(2-chloro-3-fluorobenzyl)-N-(5- | |
| | chloro-6-(pyrrolidine-1- | |
| | carbonyl)pyridin-3-yl)-5- | |
| | (trifluoromethyl)-1H-pyrazole-4- | |
| | carboxamide | |
| 46 | 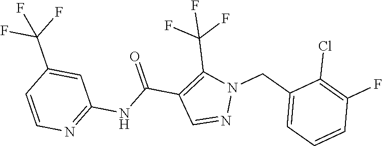 [SMILES: O=C(Nc1cc(C(F)(F)F)ccn1)c1cnn(Cc2cccc(F)c2Cl)c1C(F)(F)F] | ¹H NMR (400 MHz, DMSO-d₆) δ 11.56 (s, 1H), 8.67 (d, J = 5.1 Hz, 1H), 8.45 (d, J = 1.3 Hz, 1H), 8.27 (s, 1H), 7.62-7.33 (m, 3H), 6.75 (dd, J = 6.8, 1.8 Hz, 1H), 5.74 (d, J = 12.9 Hz, 2H).; LCMS: 467.10 [M + H]⁺. |
| | 1-(2-chloro-3-fluorobenzyl)-5- | |
| | (trifluoromethyl)-N-(4- | |
| | (trifluoromethyl)pyridin-2-yl)-1H- | |
| | pyrazole-4-carboxamide | |
| 47 | 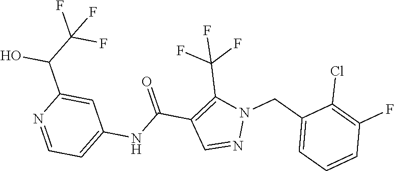 [SMILES: O=C(Nc1ccnc(C(O)C(F)(F)F)c1)c1cnn(Cc2cccc(F)c2Cl)c1C(F)(F)F] | ¹H NMR (300 MHz, DMSO-d₆ + D₂O) δ 8.33 (s, 1H), 7.97 (d, J = 5.8 Hz, 1H), 7.37 (t, J = 6.7, 6.7 Hz, 2H), 6.73 (d, J = 5.8 Hz, 2H), 6.52 (dd, J = 5.6, 2.2 Hz, 1H), 6.22 (q, J = 7.0, 6.9, 6.9 Hz, 1H), 5.76 (s, 2H).; LCMS: 497.20 [M + H]⁺. |
| | 1-(2-chloro-3-fluorobenzyl)-N-(2- | |
| | (2,2,2-trifluoro-1- | |
| | hydroxyethyl)pyridin-4-y1)-5- | |
| | (trifluoromethyl)-1H-pyrazole-4- | |
| | carboxamide | |
| 48 | 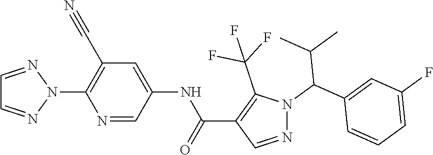 [SMILES: CC(C)C(c1cccc(F)c1)n1ncc(C(=O)Nc2cnc(-n3nccn3)c(C#N)c2)c1C(F)(F)F] | ¹H NMR (300 MHz, DMSO-d₆) δ 11.21 (d, J = 4.4 Hz, 1H), 8.97 (dd, J = 5.8, 2.5 Hz, 1H), 8.83-8.68 (m, 1H), 8.26 (d, J = 14.9 Hz, 2H), 8.05 (s, 1H), 7.49-7.33 (m, 3H), 7.18 (t, J = 8.3, 8.3 Hz, 1H), 5.12 (d, J = 10.4 Hz, 1H), 1.23 (d, J = 6.0 Hz, 1H), 0.81 (t, J = 7.3, 7.3 Hz, 6H).; LCMS: 499.05 [M + H]⁺. |
| | N-(5-cyano-6-(2H-1,2,3-triazol-2- | |
| | yl)pyridin-3-yl)-1-(1-(3- | |
| | fluorophenyl)-2-methylpropyl)-5- | |
| | (trifluoromethyl)-1H-pyrazole-4- | |
| | carboxamide | |
| 49 | 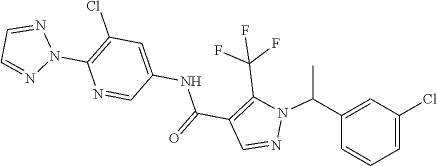 [SMILES: CC(c1cccc(Cl)c1)n1ncc(C(=O)Nc2cnc(-n3nccn3)c(Cl)c2)c1C(F)(F)F] | ¹H NMR (300 MHz, DMSO-d₆) δ 11.18 (s, 1H), 8.84-8.72 (m, 1H), 8.59 (t, J = 1.7, 1.7 Hz, 1H), 8.32-8.09 (m, 3H), 7.51-7.32 (m, 3H), 7.25 (td, J= 6.0, 5.8, 4.0 Hz, 1H), 5.93 (q, J = 6.8, 6.8, 6.8 Hz, 1H), 1.89 (d, J = 6.7 Hz, 3H).; LCMS: 497.45 [M + H]⁺. |
| | N-(5-chloro-6-(2H-1,2,3-triazol-2- | |
| | yl)pyridin-3-yl)-1-(1-(3- | |
| | chlorophenyl)ethyl)-5- | |
| | (trifluoromethyl)-1H-pyrazole-4- | |
| | carboxamide | |
| 50 | 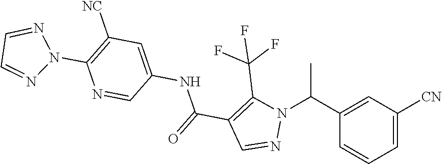 [SMILES: CC(c1cccc(C#N)c1)n1ncc(C(=O)Nc2cnc(-n3nccn3)c(C#N)c2)c1C(F)(F)F] | ¹H NMR (300 MHz, DMSO-d₆) δ 11.22 (s, 1H), 9.00 (d, J = 2.5 Hz, 1H), 8.79 (d, J = 2.5 Hz, 1H), 8.29 (d, J = 9.1 Hz, 3H), 7.95-7.71 (m, 2H), 7.61 (d, J = 4.7 Hz, 2H), 6.00 (q, J = 6.9, 6.9, 6.9 Hz, 1H), 1.91 (d, J = 6.8 Hz, 3H).; LCMS: 478.3 [M + H]⁺. |
| | N-(5-cyano-6-(2H-1,2,3-triazol-2- | |
| | yl)pyridin-3-yl)-1-(1-(3- | |
| | cyanophenyl)ethyl)-5- | |
| | (trifluoromethyl)-1H-pyrazole-4- | |
| | carboxamide | |
| 51 | 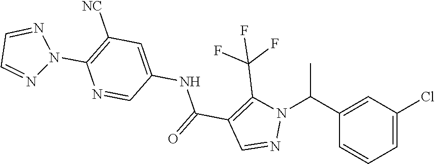 [SMILES: CC(c1cccc(Cl)c1)n1ncc(C(=O)Nc2cnc(-n3nccn3)c(C#N)c2)c1C(F)(F)F] | ¹H NMR (300 MHz, DMSO-d₆) δ 11.23 (s, 1H), 9.00 (d, J = 2.6 Hz, 1H), 8.79 (d, J = 2.5 Hz, 1H), 8.28 (d, J = 12.1 Hz, 3H), 7.40 (t, J = 8.3, 8.3 Hz, 3H), 7.24 (d, J = 6.5 Hz, 1H), 5.93 (q, J = 6.9, 6.9, 6.9 Hz, 1H), 1.89 (d, J = 6.8 Hz, 3H).; LCMS: 487.55 [M + H]⁺. |
| | 1-(1-(3-chlorophenyl)ethyl)-N-(5- | |
| | cyano-6-(2H-1,2,3-triazol-2- | |
| | y1)pyridin-3-y1)-5- | |
| | (trifluoromethyl)-1H-pyrazole-4- | |
| | carboxamide | |
| 52 | 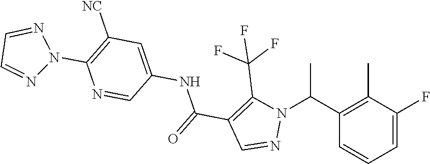 [SMILES: Cc1c(F)cccc1C(C)n1ncc(C(=O)Nc2cnc(-n3nccn3)c(C#N)c2)c1C(F)(F)F] | ¹H NMR (300 MHz, DMSO-d₆) δ 11.17 (s, 1H), 9.00 (d, J = 2.5 Hz, 1H), 8.78 (d, J = 2.5 Hz, 1H), 8.28 (d, J = 3.0 Hz, 3H), 7.37-7.01 (m, 2H), 6.73 (d, J = 7.7 Hz, 1H), 6.10 (q, J = 6.7, 6.7, 6.7 Hz, 1H), 2.22 (d, J = 2.0 Hz, 3H), 1.82 (d, J = 6.7 Hz, 3H).; LCMS: 485.2 [M + H]⁺. |
| | N-(5-cyano-6-(2H-1,2,3-triazol-2- | |
| | yl)pyridin-3-yl)-1-(1-(3-fluoro-2- | |
| | methylphenyl)ethyl)-5- | |
| | (trifluoromethyl)-1H-pyrazole-4- | |
| | carboxamide | |
| 53 | 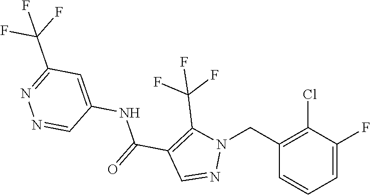 [SMILES: O=C(Nc1cnnc(C(F)(F)F)c1)c1cnn(Cc2cccc(F)c2Cl)c1C(F)(F)F] | 1H-NMR (300 MHz, DMSO-d₆) δ 11.2 (s, 1H), 9.60 (s, 1H), 8.47 (s, 1H), 8.30 (s, 1H), 7.45-7.41 (m, 2H), 6.78 (d, J = 6.6 Hz, 1H), 5.8 (s, 2H).; LCMS: 468.1 [M + H]⁺. |
| | 1-(2-chloro-3-fluorobenzyl)-5- | |
| | (trifluoromethyl)-N-(6- | |
| | (trifluoromethyl)pyridazin-4-yl)- | |
| | 1H-pyrazole-4-carboxamide | |
| 54 | 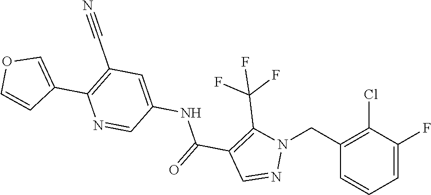 [SMILES: N#Cc1cc(NC(=O)c2cnn(Cc3cccc(F)c3Cl)c2C(F)(F)F)cnc1-c1ccoc1] | ¹H NMR (300 MHz, Methanol-d₄) δ 8.98 (d, J = 3.0 Hz, 1H), 8.61 (d, J = 2.7 Hz, 1H), 8.38 (s, 1H), 8.09 (s, 1H), 7.66 (s, 1H), 7.32-7.25 (m, 2H), 7.17 (d, J = 1.5 Hz, 1H), 6.65 (d, J = 7.5 Hz, 1H), 5.74 (s, 2H).; LCMS: 490.1 [M + H]⁺. |
| | 1-(2-chloro-3-fluorobenzyl)-N-(5- | |
| | cyano-6-(furan-3-yl)pyridin-3-y1)- | |
| | 5-(trifluoromethyl)-1H-pyrazole-4- | |
| | carboxamide | |
| 55 | 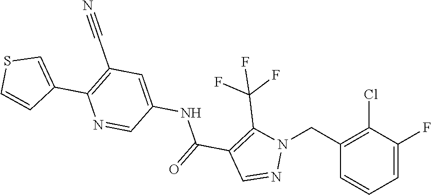 [SMILES: N#Cc1cc(NC(=O)c2cnn(Cc3cccc(F)c3Cl)c2C(F)(F)F)cnc1-c1ccsc1] | ¹H NMR (300 MHz, Methanol-d₄) δ 9.0 (d, J = 2.7 Hz, 1H), 8.64 (d, J = 2.7 Hz, 1H), 8.24 (d, J = 2.1 Hz, 1H), 8.10 (s, 1H), 7.81-7.79 (m, 1H), 7.58-7.55 (m, 1H), 7.32-7.25 (m, 2H), 6.65 (d, J = 7.2 Hz, 1H), 5.78 (s, 2H).; LCMS: 506.1 [M + H]⁺. |
| | 1-(2-chloro-3-fluorobenzyl)-N-(5- | |
| | cyano-6-(thiophen-3-y1)pyridin-3- | |
| | y1)-5-(trifluoromethyl)-1H- | |
| | pyrazole-4-carboxamide | |
| 56 | 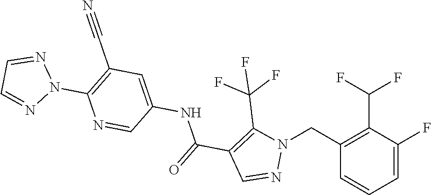 [SMILES: N#Cc1cc(NC(=O)c2cnn(Cc3cccc(F)c3C(F)F)c2C(F)(F)F)cnc1-n1nccn1] | ¹H NMR (300 MHz, DMSO-d₆) δ 8.84 (d, J = 8.4 Hz, 2H), 8.26 (s, 2H), 8.14 (s, 1H), 7.64 (s, 1H), 7.37-7.25 (m, 4H), 5.70 (s, 2H).; LCMS: 507.0 [M + H]⁺. |
| | N-(5-cyano-6-(2H-1,2,3-triazol-2- | |
| | yl)pyridin-3-y1)-1-(2- | |
| | (difluoromethyl)-3-fluorobenzyl)-5- | |
| | (trifluoromethyl)-1H-pyrazole-4- | |
| | carboxamide | |

### Example-P1

### Biochemical Assay

The potency of compounds to inhibit MALT-1 (Isoform 1) enzyme was tested in a Fluorescent assay using recombinant MALT-1 (Isoform 1, aa-840) generated in house. The assay buffer was 50 mM HEPES (pH 7.5), 100 mM NaCl, 10 mM DTT, 1 mM EDTA, 0.9 M sodium citrate, 0.01% CHAPS. 1.5 nM of MALT-1 enzyme was incubated with test compounds (1 p.M and 10 p.M) (1% DMSO) in the presence of buffer for 30 minutes at 30° C. The protease reaction was initiated by adding 50 p.M of AC-LRSR-MAC (Peptide International, USA) substrate and incubated for 240 min. After 240 min incubation, fluorescence emission of the samples at 460 nm was measured at an excitation of 355 nm. The percent inhibition was calculated using the following formula: (Control OD—(Sample OD/Control OD))×100. IC₅₀ values were determined by fitting the dose-response data to sigmoidal curve fitting equation using Graph pad prism software V8.

Selected compounds of the present invention were screened in the above-mentioned assay procedure for the determination of IC₅₀ values and the results are summarized into groups A, B and C in the table below. Herein group “A” refers to IC₅₀ values lower than 0.25 μM, “B” refers to IC₅₀ values between 0.251 μM and 1 μM (both inclusive) and “C” refers to IC₅₀ values higher than 1 μM. ND represents Not Determined. The results are given below.

| Compound | Percent Inhibition (1 μM) | IC₅₀ (μM) |
|---|---|---|
| 1 | 100 | A |
| 2 | 96 | A |
| 2a | 99 | A |
| 2b | 57 | B |
| 3 | 83 | A |
| 3a | 76 | B |
| 3b | 100 | A |
| 4 | 77 | B |
| 4a | 83 (10 μM) | C |
| 4b | 94 | A |
| 5 | 89 | B |
| 5a | 78 | B |
| 5b | 99 | A |
| 6 | 81 | B |
| 7 | 88 | B |
| 8 | 87 | A |
| 9 | 55 | B |
| 10 | 44 | C |
| 12 | 98 | A |
| 13 | 40 | C |
| 14 | 67 | C |
| 15 | 99 | A |
| 17 | 48 (10 μM) | ND |
| 18 | 47 | C |
| 19 | 22 | C |
| 20 | 46 | C |
| 21 | 23 | C |
| 22 | 41 | ND |
| 23 | 45 | C |
| 25 | 100 | A |
| 26 | 100 | A |
| 27 | 100 | A |
| 28 | 84 | B |
| 29 | 70 | B |
| 30 | 97 | B |
| 31 | 95 | A |
| 32 | 99 | A |
| 33 | 99 | A |
| 34 | 99 | A |
| 35 | 42 | C |
| 36 | 86 | B |
| 37 | 81 | B |
| 38 | 100 | A |
| 39 | 100 | A |
| 40 | 75 | B |
| 41 | 14 | C |
| 42 | 71 | B |
| 43 | 13 | C |
| 44 | 38 | C |
| 45 | 40 | C |
| 46 | 32 | C |
| 47 | 33 (10 μM) | ND |
| 48 | 23 | C |
| 49 | 88 | B |
| 50 | 65 | A |
| 51 | 100 | A |
| 52 | 100 | A |
| 53 | 60 | B |
| 54 | 77 | B |
| 55 | 63 | B |
| 56 | 77 | B |

### INCORPORATION BY REFERENCE

All publications and patents mentioned herein are hereby incorporated by reference in their entirety as if each individual publication or patent were specifically and individually indicated to be incorporated by reference. In case of conflict, the present application, including any definitions herein, will control.

### Equivalents

While specific embodiments of the subject invention have been discussed, the above specification is only illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound of formula (I) 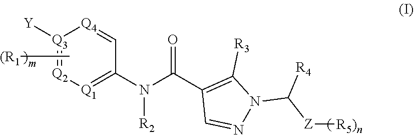
or a pharmaceutically acceptable salt or a stereoisomer thereof;
wherein;
each Q₁, Q₂, Q_{3,} and Q₄ independently represents N, CH or C, wherein at least one of Q₁, Q₂, Q₃ and Q₄ is N;
Y is absent or 5- to 6-membered heteroaryl;
R₁, at each occurrence, is hydroxy, hydroxyalkyl, halogen, alkoxy, alkyl, haloalkyl, haloalkoxy, amino, alkylamino, cyano or —CO—(5- to 6-membered heterocycloalkyl); wherein the said alkyl and haloalkyl, at each occurrence, are optionally substituted with 1 or 2 substituents independently selected from hydroxy, alkoxy and amino;
R₂ is hydrogen, halogen or alkyl;
Z represents phenyl or 5- to 6-membered heteroaryl;
R₃ is alkyl, haloalkyl, 3- to 10-membered cycloalkyl, 6- to 10-membered aryl or 5- to 12-membered heteroaryl;
R₄ is hydrogen, alkyl, haloalkyl or 3- to 10-membered cycloalkyl;
R₅, at each occurrence, is hydrogen, hydroxy, hydroxyalkyl, halogen, alkoxy, alkyl, haloalkyl, haloalkoxy, amino, alkylamino or cyano; or any two R₅ groups attached to adjacent carbon atoms of ring Z combine together to form Z₁ ring;
Z₁ represents fused benzo, fused 5- to 6-membered heteroaryl or fused 5- to 6-membered heterocycloalkyl ring;
m is 0, 1, 2 or 3, and
n is 1, 2 or 3.

2. The compound of claim 1, wherein Y represents thienyl, furanyl, pyrrolyl, imidazolyl, pyrazolyl, isoxazolyl, oxazolyl, isothiazolyl, thiazolyl, 1H-tetrazolyl, triazolyl, oxadiazolyl, pyridazinyl, pyridyl, pyrimidinyl or pyrazinyl.

3. (canceled)

4. The compound of claim 1, wherein R₃ is haloalkyl or 3- to 10-membered cycloalkyl.

5. (canceled)

6. (canceled)

7. (canceled)

8. (canceled)

9. (canceled)

10. The compound of claim 1, wherein any two R₅ groups attached to adjacent carbon atoms of ring Z combine together to form Z₁ ring.

11. The compound of claim 10, wherein Z₁ represents 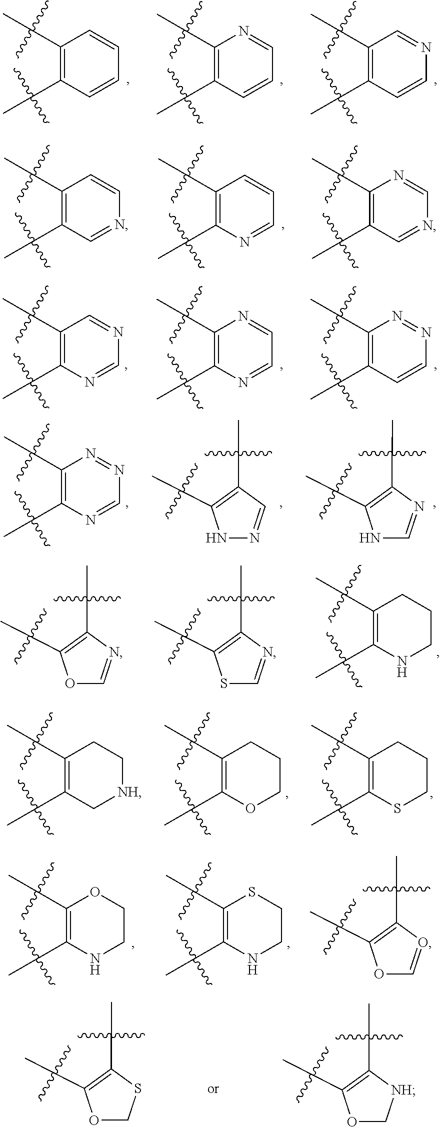
wherein [symbol] represents the points of fusion with Z.

12. The compound of claim 1, wherein
each Q₁, Q₂, Q₃, and Q₄ independently represents N, CH or C, wherein at least one of Q₁, Q₂, Q₃ and Q₄ is N;
Y is absent or 5- to 6-membered heteroaryl;
R₁, at each occurrence, is hydroxy, hydroxyalkyl, halogen, alkoxy, —CN, pyrrolidinyl-CO— or haloalkyl optionally substituted with —OH;
R₂ is hydrogen;
Z represents phenyl, furanyl, thienyl, pyrrolyl, pyrazolyl, imidazolyl, oxazolyl, isoxazolyl, thiazolyl, isothiazolyl, 1H-tetrazolyl, oxadiazolyl, triazolyl, pyridyl, pyrimidinyl, pyrazinyl or pyridazinyl;
R₃ is alkyl, haloalkyl or cyclopropyl;
R₄ is hydrogen or (C₁-C₆) alkyl;
R₅, at each occurrence, is hydrogen, halogen, alkoxy, alkyl, haloalkyl or cyano; or any two R₅ groups attached to adjacent carbon atoms combine together to form Z₁ ring;
Z₁ represents 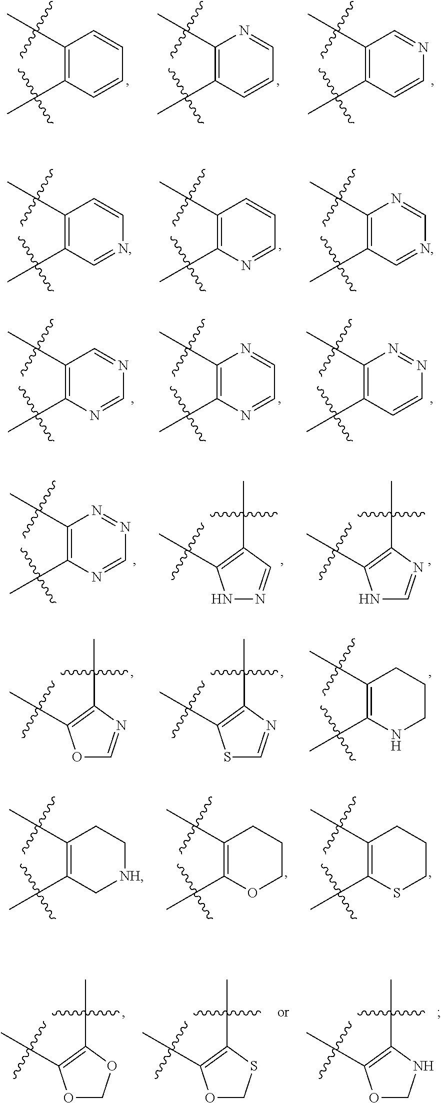
wherein [symbol] represents the points of fusion with Z;
m is 0, 1, 2 or 3, and
n is 1, 2 or 3.

13. The compound of claim 1, represented by compound of formula (IA): 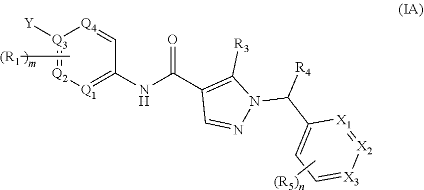
or a pharmaceutically acceptable salt or a stereoisomer thereof;
wherein, each X₁, X₂ and X₃ independently represents N or C.

14. (canceled)

15. (canceled)

16. The compound of claim 13, wherein R₄ is hydrogen, —CH₃, —CH₂CH₃ or —CH(CH₃)₂.

17. The compound of claim 13, wherein
—X₁—X₂—X₃— represents —C—C—C—, —N—C—C, —C—N—C or —C—C—N—;
each Q₁, Q₂, Q₃, and Q₄ independently represents N, CH or C, wherein at least one of Q₁, Q₂, Q₃ and Q₄ is N;
R₁, at each occurrence, is —CN, —Cl, —CF₃, —O—CH₃, —F, pyrrolidinyl-CO— or —CH(OH)CF₃;
R₃ is —CF₃ or cyclopropyl;
R₄ is hydrogen, —CH₃, —CH₂CH₃ or —CH(CH₃)₂;
R₅, at each occurrence, is —Cl, —F, —Br, —CN, —CF₃, —OCH₃ or —CH₃; or any two R₅ groups attached to adjacent carbon atoms combine together to form Z₁ ring;
Z₁ represents 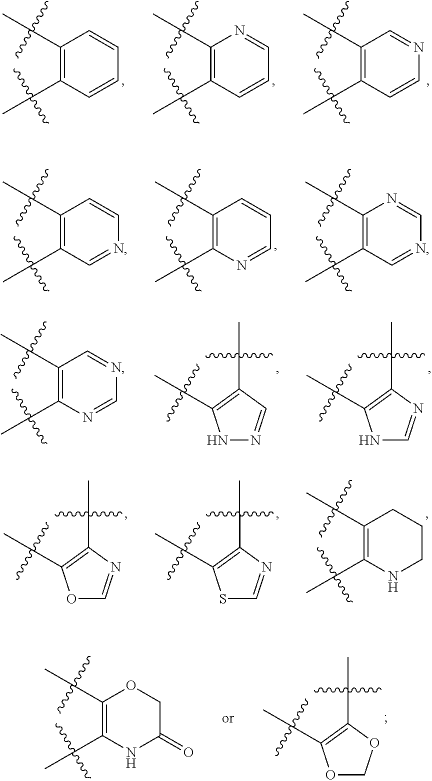
m is 0, 1, 2 or 3, and
n is 1, 2 or 3.

18. The compound of claim 1, represented by compound of formula (IB); 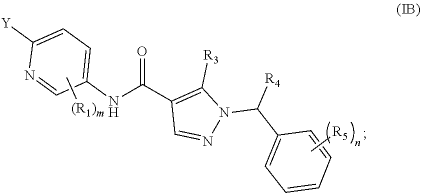
or a pharmaceutically acceptable salt or a stereoisomer thereof.

19. The compound of claim 18, wherein
R₁, at each occurrence, is halogen, alkoxy, haloalkyl, —CN or pyrrolidinyl-CO—;
R₃ is haloalkyl or 3- to 10-membered cycloalkyl;
R₄ is hydrogen or alkyl;
R₅, at each occurrence, is hydrogen, halogen, haloalkyl, alkyl, cyano, hydroxy or alkoxy; or any two R₅ groups attached to adjacent carbon atoms combine together to form Z₁ ring; and
Z₁ represents fused benzo, fused 5- to 6-membered heteroaryl or fused 5- to 6-membered heterocycloalkyl ring.

20. The compound of claim 18, wherein
R₁, at each occurrence, is —CN, —Cl, —CF₃, —O—CH₃, —F or pyrrolidinyl-CO—;
R₃ is —CF₃ or cyclopropyl;
R₄ is hydrogen, —CH₃, —CH₂CH₃ or —CH(CH₃)₂;
R₅, at each occurrence, is —Cl, —F, —Br, —CN, —CF₃, —OCH₃ or —CH₃; or any two R₅ groups attached to adjacent carbon atoms combine together to form Z₁ ring;
Z₁ represents

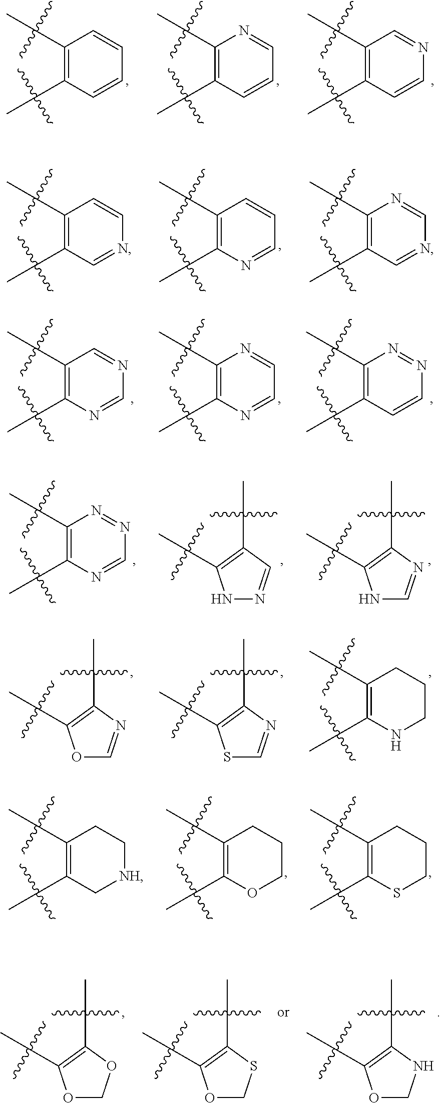

21. The compound of claim 1, represented by compound of formula (IC);

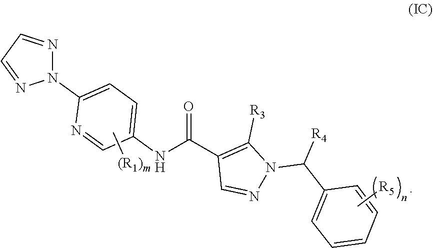

22. The compound of claim 21, wherein
R₁ at each occurrence, is —CN, —Cl, —CF₃, —O—CH₃ or —F;
R₃ is —CF₃ or cyclopropyl;
R₄ is hydrogen, —CH₃, —CH₂CH₃ or —CH(CH₃)₂;
R₅, at each occurrence, is —Cl, —F, —Br, —CN, —CF₃, —OCH₃ or —CH₃; or any two R₅ groups attached to adjacent carbon atoms combine together to form Z₁ ring;
Z₁ represents 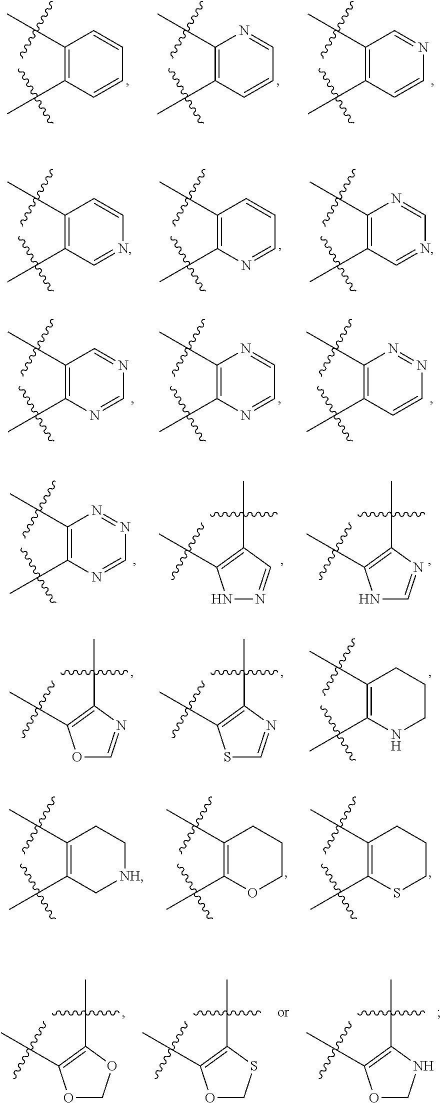
wherein [symbol] represents the points of fusion with Z;
m is 1, 2 or 3, and
n is 1, 2 or 3.

23. The compound of claim 1, is selected from

| Compound | Structure |
|---|---|
| 1 | 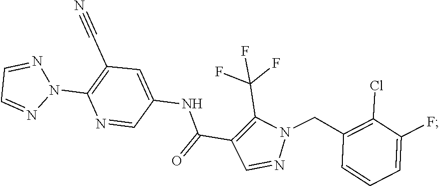 [SMILES: N#Cc1cc(NC(=O)c2cnn(Cc3cccc(F)c3Cl)c2C(F)(F)F)cnc1-n1nccn1] |
| 2 | 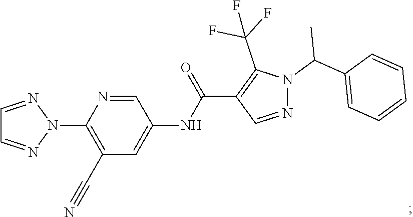 [SMILES: CC(c1ccccc1)n1ncc(C(=O)Nc2cnc(-n3nccn3)c(C#N)c2)c1C(F)(F)F] |
| 2a | 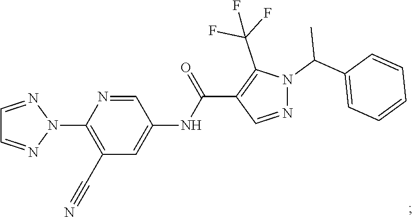 [SMILES: CC(c1ccccc1)n1ncc(C(=O)Nc2cnc(-n3nccn3)c(C#N)c2)c1C(F)(F)F] |
| 2b | 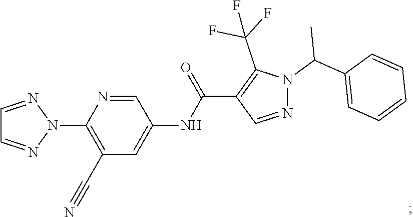 [SMILES: CC(c1ccccc1)n1ncc(C(=O)Nc2cnc(-n3nccn3)c(C#N)c2)c1C(F)(F)F] |
| 3 | 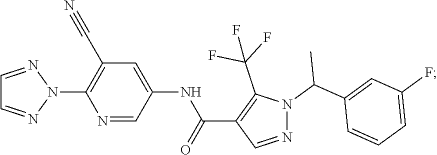 [SMILES: CC(c1cccc(F)c1)n1ncc(C(=O)Nc2cnc(-n3nccn3)c(C#N)c2)c1C(F)(F)F] |
| 3a | 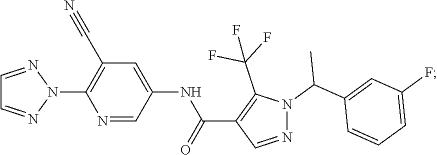 [SMILES: CC(c1cccc(F)c1)n1ncc(C(=O)Nc2cnc(-n3nccn3)c(C#N)c2)c1C(F)(F)F] |
| 3b | 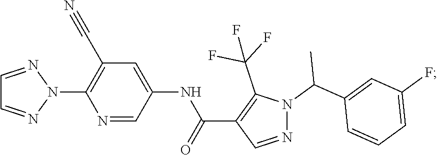 [SMILES: CC(c1cccc(F)c1)n1ncc(C(=O)Nc2cnc(-n3nccn3)c(C#N)c2)c1C(F)(F)F] |
| 4 | 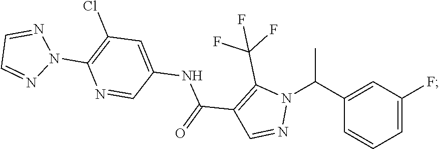 [SMILES: CC(c1cccc(F)c1)n1ncc(C(=O)Nc2cnc(-n3nccn3)c(Cl)c2)c1C(F)(F)F] |
| 4a | 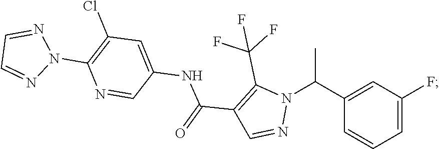 [SMILES: CC(c1cccc(F)c1)n1ncc(C(=O)Nc2cnc(-n3nccn3)c(Cl)c2)c1C(F)(F)F] |
| 4b | 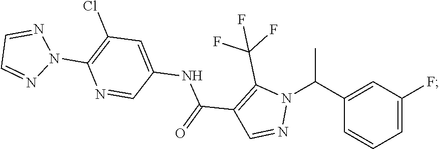 [SMILES: CC(c1cccc(F)c1)n1ncc(C(=O)Nc2cnc(-n3nccn3)c(Cl)c2)c1C(F)(F)F] |
| 5a | 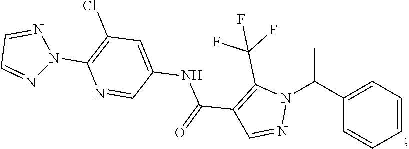 [SMILES: CC(c1ccccc1)n1ncc(C(=O)Nc2cnc(-n3nccn3)c(Cl)c2)c1C(F)(F)F] |
| 5b | 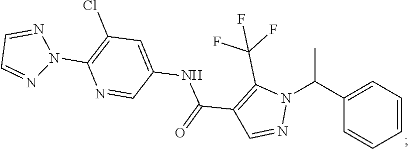 [SMILES: CC(c1ccccc1)n1ncc(C(=O)Nc2cnc(-n3nccn3)c(Cl)c2)c1C(F)(F)F] |
| 6 | 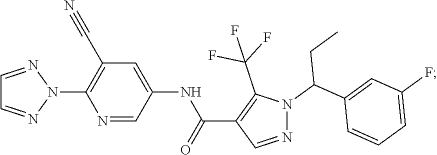 [SMILES: CCC(c1cccc(F)c1)n1ncc(C(=O)Nc2cnc(-n3nccn3)c(C#N)c2)c1C(F)(F)F] |
| 7 | 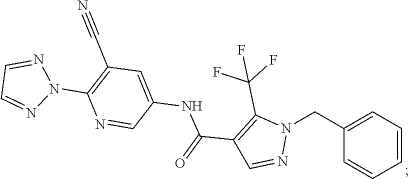 [SMILES: N#Cc1cc(NC(=O)c2cnn(Cc3ccccc3)c2C(F)(F)F)cnc1-n1nccn1] |
| 8 | 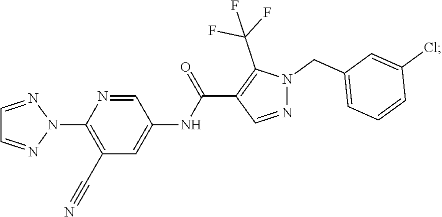 [SMILES: N#Cc1cc(NC(=O)c2cnn(Cc3cccc(Cl)c3)c2C(F)(F)F)cnc1-n1nccn1] |
| 9 | 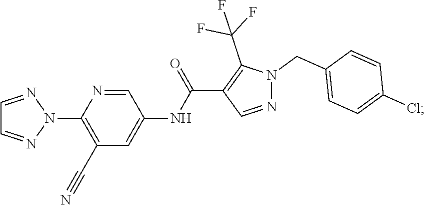 [SMILES: N#Cc1cc(NC(=O)c2cnn(Cc3ccc(Cl)cc3)c2C(F)(F)F)cnc1-n1nccn1] |
| 10 | 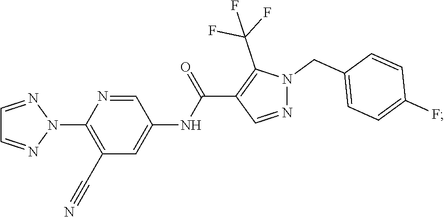 [SMILES: N#Cc1cc(NC(=O)c2cnn(Cc3ccc(F)cc3)c2C(F)(F)F)cnc1-n1nccn1] |
| 11 | 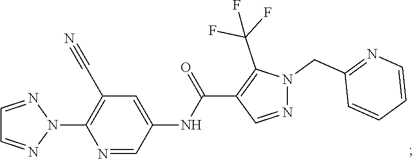 [SMILES: N#Cc1cc(NC(=O)c2cnn(Cc3ccccn3)c2C(F)(F)F)cnc1-n1nccn1] |
| 12 | 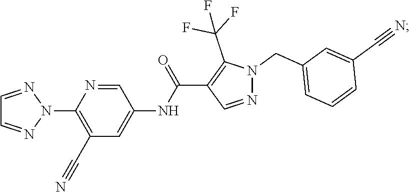 [SMILES: N#Cc1cccc(Cn2ncc(C(=O)Nc3cnc(-n4nccn4)c(C#N)c3)c2C(F)(F)F)c1] |
| 13 | 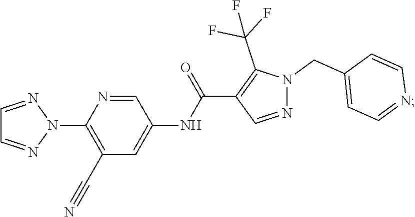 [SMILES: N#Cc1cc(NC(=O)c2cnn(Cc3ccncc3)c2C(F)(F)F)cnc1-n1nccn1] |
| 14 | 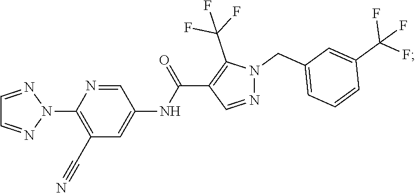 [SMILES: N#Cc1cc(NC(=O)c2cnn(Cc3cccc(C(F)(F)F)c3)c2C(F)(F)F)cnc1-n1nccn1] |
| 15 | 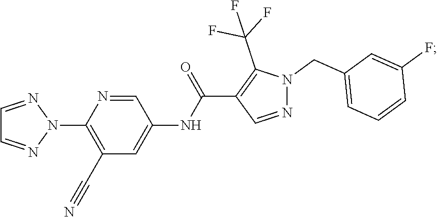 [SMILES: N#Cc1cc(NC(=O)c2cnn(Cc3cccc(F)c3)c2C(F)(F)F)cnc1-n1nccn1] |
| 16 | 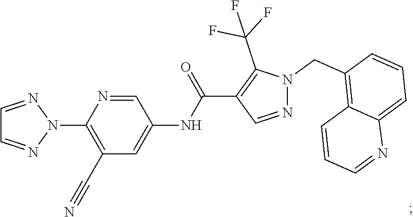 [SMILES: N#Cc1cc(NC(=O)c2cnn(Cc3cccc4ncccc34)c2C(F)(F)F)cnc1-n1nccn1] |
| 17 | 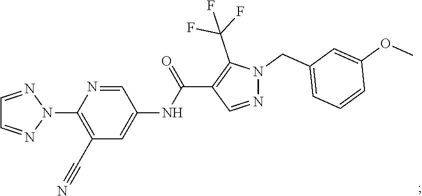 [SMILES: COc1cccc(Cn2ncc(C(=O)Nc3cnc(-n4nccn4)c(C#N)c3)c2C(F)(F)F)c1] |
| 18 | 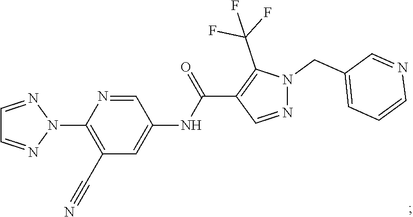 [SMILES: N#Cc1cc(NC(=O)c2cnn(Cc3cccnc3)c2C(F)(F)F)cnc1-n1nccn1] |
| 19 | 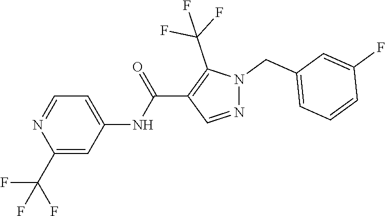 [SMILES: O=C(Nc1ccnc(C(F)(F)F)c1)c1cnn(Cc2cccc(F)c2)c1C(F)(F)F] |
| 20 | 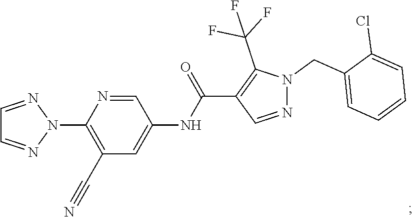 [SMILES: N#Cc1cc(NC(=O)c2cnn(Cc3ccccc3Cl)c2C(F)(F)F)cnc1-n1nccn1] |
| 21 | 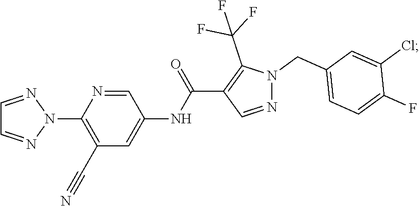 [SMILES: N#Cc1cc(NC(=O)c2cnn(Cc3ccc(F)c(Cl)c3)c2C(F)(F)F)cnc1-n1nccn1] |
| 22 | 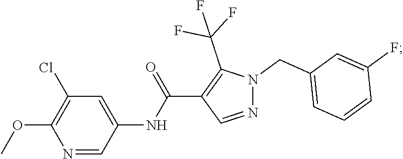 [SMILES: COc1ncc(NC(=O)c2cnn(Cc3cccc(F)c3)c2C(F)(F)F)cc1Cl] |
| 23 | 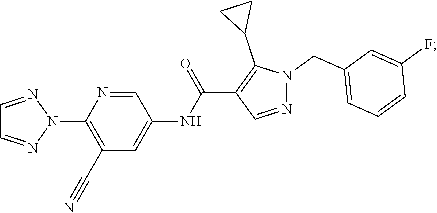 [SMILES: N#Cc1cc(NC(=O)c2cnn(Cc3cccc(F)c3)c2C2CC2)cnc1-n1nccn1] |
| 24 | 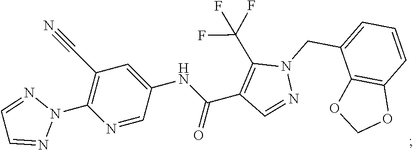 [SMILES: N#Cc1cc(NC(=O)c2cnn(Cc3cccc4c3OCO4)c2C(F)(F)F)cnc1-n1nccn1] |
| 25 | 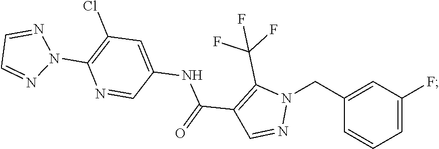 [SMILES: O=C(Nc1cnc(-n2nccn2)c(Cl)c1)c1cnn(Cc2cccc(F)c2)c1C(F)(F)F] |
| 26 | 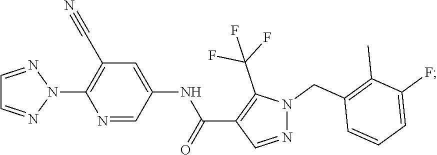 [SMILES: Cc1c(F)cccc1Cn1ncc(C(=O)Nc2cnc(-n3nccn3)c(C#N)c2)c1C(F)(F)F] |
| 27 | 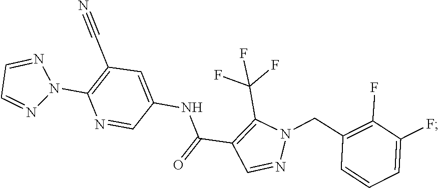 [SMILES: N#Cc1cc(NC(=O)c2cnn(Cc3cccc(F)c3F)c2C(F)(F)F)cnc1-n1nccn1] |
| 28 | 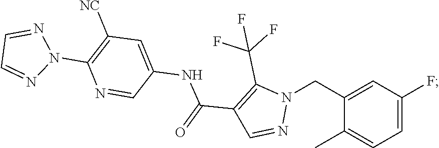 [SMILES: Cc1ccc(F)cc1Cn1ncc(C(=O)Nc2cnc(-n3nccn3)c(C#N)c2)c1C(F)(F)F] |
| 29 | 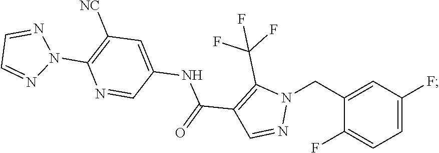 [SMILES: N#Cc1cc(NC(=O)c2cnn(Cc3cc(F)ccc3F)c2C(F)(F)F)cnc1-n1nccn1] |
| 30 | 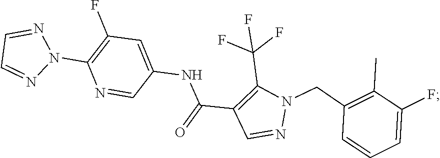 [SMILES: Cc1c(F)cccc1Cn1ncc(C(=O)Nc2cnc(-n3nccn3)c(F)c2)c1C(F)(F)F] |
| 31 | 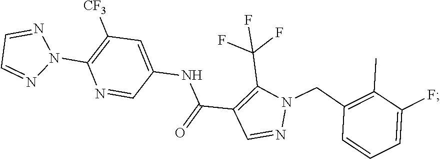 [SMILES: Cc1c(F)cccc1Cn1ncc(C(=O)Nc2cnc(-n3nccn3)c(C(F)(F)F)c2)c1C(F)(F)F] |
| 32 | 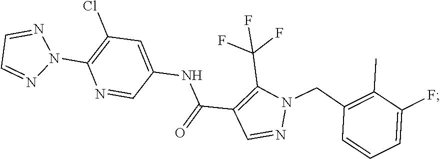 [SMILES: Cc1c(F)cccc1Cn1ncc(C(=O)Nc2cnc(-n3nccn3)c(Cl)c2)c1C(F)(F)F] |
| 33 | 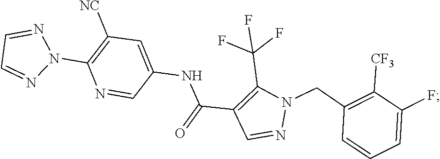 [SMILES: N#Cc1cc(NC(=O)c2cnn(Cc3cccc(F)c3C(F)(F)F)c2C(F)(F)F)cnc1-n1nccn1] |
| 34 | 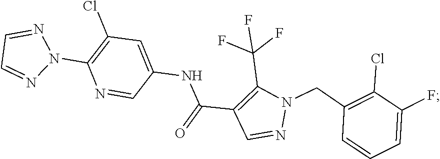 [SMILES: O=C(Nc1cnc(-n2nccn2)c(Cl)c1)c1cnn(Cc2cccc(F)c2Cl)c1C(F)(F)F] |
| 35 | 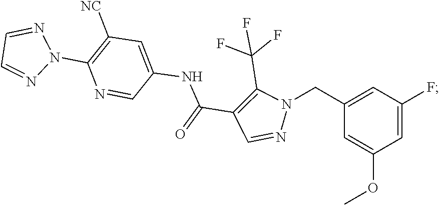 [SMILES: COc1cc(F)cc(Cn2ncc(C(=O)Nc3cnc(-n4nccn4)c(C#N)c3)c2C(F)(F)F)c1] |
| 36 | 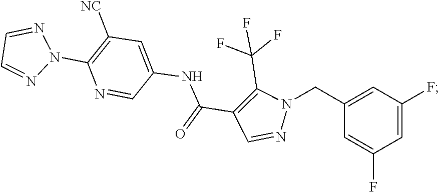 [SMILES: N#Cc1cc(NC(=O)c2cnn(Cc3cc(F)cc(F)c3)c2C(F)(F)F)cnc1-n1nccn1] |
| 37 | 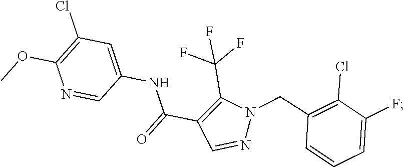 [SMILES: COc1ncc(NC(=O)c2cnn(Cc3cccc(F)c3Cl)c2C(F)(F)F)cc1Cl] |
| 38 | 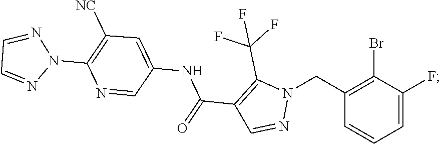 [SMILES: N#Cc1cc(NC(=O)c2cnn(Cc3cccc(F)c3Br)c2C(F)(F)F)cnc1-n1nccn1] |
| 39 | 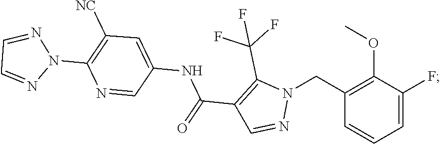 [SMILES: COc1c(F)cccc1Cn1ncc(C(=O)Nc2cnc(-n3nccn3)c(C#N)c2)c1C(F)(F)F] |
| 40 | 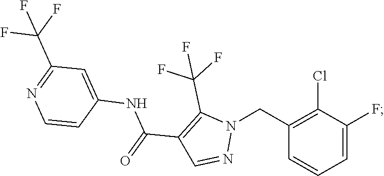 [SMILES: O=C(Nc1ccnc(C(F)(F)F)c1)c1cnn(Cc2cccc(F)c2Cl)c1C(F)(F)F] |
| 41 | 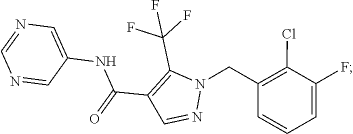 [SMILES: O=C(Nc1cncnc1)c1cnn(Cc2cccc(F)c2Cl)c1C(F)(F)F] |
| 42 | 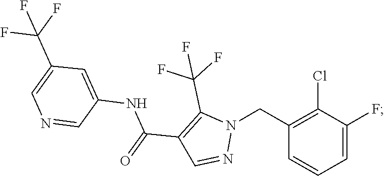 [SMILES: O=C(Nc1cncc(C(F)(F)F)c1)c1cnn(Cc2cccc(F)c2Cl)c1C(F)(F)F] |
| 43 | 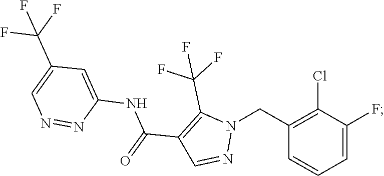 [SMILES: O=C(Nc1cc(C(F)(F)F)cnn1)c1cnn(Cc2cccc(F)c2Cl)c1C(F)(F)F] |
| 44 | 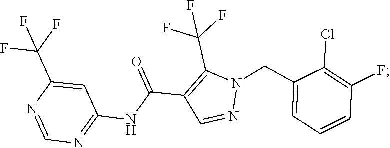 [SMILES: O=C(Nc1cc(C(F)(F)F)ncn1)c1cnn(Cc2cccc(F)c2Cl)c1C(F)(F)F] |
| 45 | 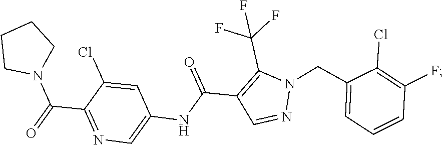 [SMILES: O=C(Nc1cnc(C(=O)N2CCCC2)c(Cl)c1)c1cnn(Cc2cccc(F)c2Cl)c1C(F)(F)F] |
| 46 | 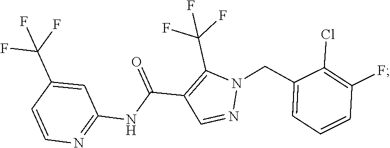 [SMILES: O=C(Nc1cc(C(F)(F)F)ccn1)c1cnn(Cc2cccc(F)c2Cl)c1C(F)(F)F] |
| 47 | 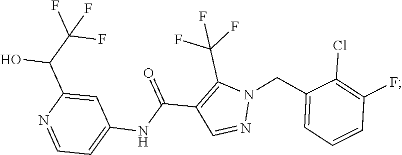 [SMILES: O=C(Nc1ccnc(C(O)C(F)(F)F)c1)c1cnn(Cc2cccc(F)c2Cl)c1C(F)(F)F] |
| 48 | 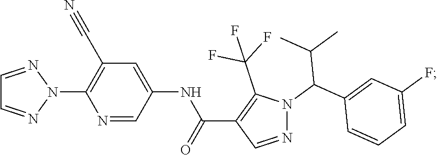 [SMILES: CC(C)C(c1cccc(F)c1)n1ncc(C(=O)Nc2cnc(-n3nccn3)c(C#N)c2)c1C(F)(F)F] |
| 49 | 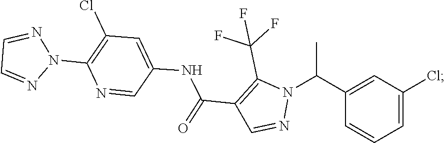 [SMILES: CC(c1cccc(Cl)c1)n1ncc(C(=O)Nc2cnc(-n3nccn3)c(Cl)c2)c1C(F)(F)F] |
| 50 | 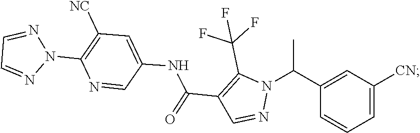 [SMILES: CC(c1cccc(C#N)c1)n1ncc(C(=O)Nc2cnc(-n3nccn3)c(C#N)c2)c1C(F)(F)F] |
| 51 | 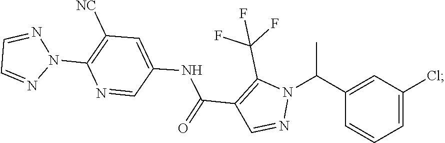 [SMILES: CC(c1cccc(Cl)c1)n1ncc(C(=O)Nc2cnc(-n3nccn3)c(C#N)c2)c1C(F)(F)F] |
| 52 | 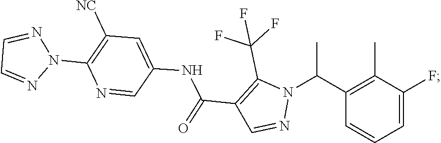 [SMILES: Cc1c(F)cccc1C(C)n1ncc(C(=O)Nc2cnc(-n3nccn3)c(C#N)c2)c1C(F)(F)F] |
| 53 | 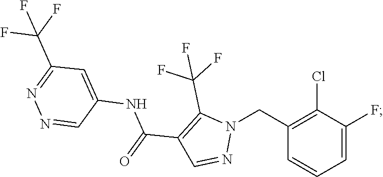 [SMILES: O=C(Nc1cnnc(C(F)(F)F)c1)c1cnn(Cc2cccc(F)c2Cl)c1C(F)(F)F] |
| 54 | 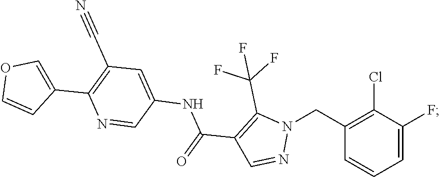 [SMILES: N#Cc1cc(NC(=O)c2cnn(Cc3cccc(F)c3Cl)c2C(F)(F)F)cnc1-c1ccoc1] |
| 55 | 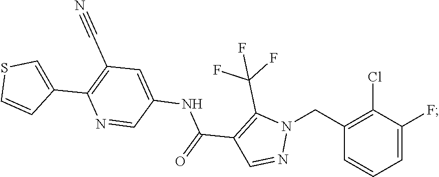 [SMILES: N#Cc1cc(NC(=O)c2cnn(Cc3cccc(F)c3Cl)c2C(F)(F)F)cnc1-c1ccsc1] |
| | and |
| 56 | 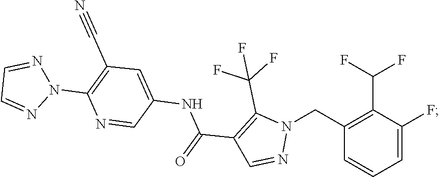 [SMILES: N#Cc1cc(NC(=O)c2cnn(Cc3cccc(F)c3C(F)F)c2C(F)(F)F)cnc1-n1nccn1] |

or a pharmaceutically acceptable salt or a stereoisomer thereof

24. A pharmaceutical composition comprising a compound of claim 1 or a pharmaceutically acceptable salt or a stereoisomer thereof, and a pharmaceutically acceptable carrier or excipient.

25. (canceled)

26. (canceled)

27. A method of inhibiting a target protein comprising contacting the target protein with a compound of claim 1 or a pharmaceutically acceptable salt or a stereoisomer thereof, wherein the compound is effective for inhibiting the target protein.

28. The method of claim 27 wherein the target protein is mucosa associated lymphoid tissue lymphoma translocation protein 1 (MALT-1).

29. A method of treating a disease or disorder in a subject comprising administering to the subject in need thereof a therapeutically effective amount of a compound claim 1 or a pharmaceutically acceptable salt or a stereoisomer thereof, or a pharmaceutical composition of claim 24, wherein the disease or disorder is mediated by modulating in vivo activity of MALT-1.

30. The method of claim 29, wherein the said disease or disorder is selected from bladder cancer, colon cancer, hepatocellular cancer, or small cell lung cancer or non-small cell lung cancer, diffuse large B-cell lymphoma, mantle cell lymphoma, follicular lymphoma, and mucosa-associated lymphoid tissue lymphoma, rheumatoid arthritis, psoriatic arthritis, psoriasis, ulcerative colitis, Crohn's disease, systemic lupus erythematosus, asthma and chronic obstructive pulmonary disease.

31. (canceled)

32. (canceled)

33. (canceled)

34. (canceled)
